SEQUENCE LISTING (18 sequences)

SEQ ID NO: 1 - DNA, 50 bp, Human immunodeficiency virus
tatcacctagaactttaaatgcatgggtaaaagtagtagaagagaaggct

SEQ ID NO: 2 - RNA, 41 nt, Artificial Sequence
  crRNA for HIV
uaauuucuacucuuguagauaaugcauggguaaaaguagua

SEQ ID NO: 3 - DNA, 51 bp, Human immunodeficiency virus
cctttaacttccctcaggtcactctttggcaacgacccctcgtcacaataa

SEQ ID NO: 4 - RNA, 43 nt, Artificial Sequence
  crRNA for HIV
uaauuucuacucuuguagauacuucccucaggucacucuuugg

SEQ ID NO: 5 - RNA, 20 nt, Artificial Sequence
  Direct Repeat sequence of a crRNA recognized by Cas12
uaauuucuacucuuguagau

SEQ ID NO: 6 - RNA, 21 nt, Artificial Sequence
  Target-specific sequence including in the crRNA of SEQ ID NO2
aaugcauggguaaaaguagua

SEQ ID NO: 7 - RNA, 23 nt, Artificial Sequence
  Target-specific sequence included in the crRNA of SEQ ID NO4
acuucccucaggucacucuuugg

SEQ ID NO: 8 - DNA, 20 bp, Artificial Sequence
  Forward primer for amplication of SARS-Cov-2
ttacaaacattggccgcaaa

SEQ ID NO: 9 - DNA, 18 bp, Artificial Sequence
  Reverse primer for amplication of SARS-Cov-2
gcgcgacattccgaagaa

SEQ ID NO: 10 - DNA, 20 bp, Artificial Sequence
  crRNA for SARS-Cov-2
cccccagcgcttcagcgttc

SEQ ID NO: 11 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 12 - protein, 933 aa, Corynebacterium diphtheriae
MSVVDQADQWRRARSVQAYSLWAKSGSEDLYLRLPQHLIDAACVAEWLWNNWVSDSLKSTLSAAWRLPAEEVGRLYTFYAGTHDVGKATISFQRLVEKTSHGNYLLGPVREAGLSLQWTLNEGEGKKFPHGMASALIIAAWLEKHDIDPSSAARLSFIADAHHGFASDEELYRSHEDTLDYYPPEWLVVHAEILDSMAEITDIGETLEELADQSTPSAPAMQIMTGLVIMADWIASDEKAFPYVCDSSQHDRVVEGMSHVNLPPAWVPTDVPDNVETLFRDTFTWPDSYQVRPVQRAAVAVARAVQDPTLIIIEAPTGEGKTEAGLATSHILGQKTGAQGIFFAAPTMSTANGLFERTKNWAQCTSSRGEVASLYLAHSKNKLSLPFQSLRFTSIGEDDHLEKHGSVVASQWLSGRHRGILSDFVVGTVDQVLMMALQVRFSMLRHVGLAGKIVIIDEVHAYDAYMSQYLYLTLQWLAKYGVSVILMSATLPPQQRARLVNAYASQVCKKADASALNSDAYPLITAVNKKGISVTEVPQENSDTTIKIRRIDDSLPALGGMFSDLLVDGGIALVICNTIRRAQQAYDSLKAIFPDEVELHHAAFIATQRSEKEDALRESLGPHASRGEDRPWRRIVVATQVAEQSLDIDADVLVTDIAPIDLIIQRAGRLHRHERPHSDRPEILGQPQIFIRGINNEEISGELPEFDGGAAAIYGEKILYATVAYLPDEFHRPSDVPKLVKNVYSDTPCIPENWKEQWEQACVKAKENYEKSVRKAQTFSFPQPHMARTLRDLFKQQHSNSVDKKEESGSAQVRDAEFSIEVVALLKTEYGYHPFGREEEIENGRELTWKEAEALAGNTVRLPAQMTRRDSDFNAVIDSLEAQTPPEWQRSGLLKGQVALFFDERGEARLGRFLVRYTNERGLEVEVCPKEDA

SEQ ID NO: 13 - protein, 1154 aa, Proteocatella sphenisci
MENFKNLYPINKTLRFELRPYGKTLENFKKSGLLEKDAFKANSRRSMQAIIDEKFKETIEERLKYTEFSECDLGNMTSKDKKITDKAATNLKKQVILSFDDEIFNNYLKPDKNIDALFKNDPSNPVISTFKGFTTYFVNFFEIRKHIFKGESSGSMAYRIIDENLTTYLNNIEKIKKLPEELKSQLEGIDQIDKLNNYNEFITQSGITHYNEIIGGISKSENVKIQGINEGINLYCQKNKVKLPRLTPLYKMILSDRVSNSFVLDTIENDTELIEMISDLINKTEISQDVIMSDIQNIFIKYKQLGNLPGISYSSIVNAICSDYDNNFGDGKRKKSYENDRKKHLETNVYSINYISELLTDTDVSSNIKMRYKELEQNYQVCKENFNATNWMNIKNIKQSEKTNLIKDLLDILKSIQRFYDLFDIVDEDKNPSAEFYTWLSKNAEKLDFEFNSVYNKSRNYLTRKQYSDKKIKLNFDSPTLAKGWDANKEIDNSTIIMRKFNNDRGDYDYFLGIWNKSTPANEKIIPLEDNGLFEKMQYKLYPDPSKMLPKQFLSKIWKAKHPTTPEFDKKYKEGRHKKGPDFEKEFLHELIDCFKHGLVNHDEKYQDVFGFNLRNTEDYNSYTEFLEDVERCNYNLSFNKIADTSNLINDGKLYVFQIWSKDFSIDSKGTKNLNTIYFESLFSEENMIEKMFKLSGEAEIFYRPASLNYCEDIIKKGHHHAELKDKFDYPIIKDKRYSQDKFFFHVPMVINYKSEKLNSKSLNNRTNENLGQFTHIIGIDRGERHLIYLTVVDVSTGEIVEQKHLDEIINTDTKGVEHKTHYLNKLEEKSKTRDNERKSWEAIETIKELKEGYISHVINEIQKLQEKYNALIVMENLNYGFKNSRIKVEKQVYQKFETALIKKFNYIIDKKDPETYIHGYQLTNPITTLDKIGNQSGIVLYIPAWNTSKIDPVTGFVNLLYADDLKYKNQEQAKSFIQKIDNIYFENGEFKFDIDFSKWNNRYSISKTKWTLTSYGTRIQTFRNPQKNNKWDSAEYDLTEEFKLILNIDGTLKSQDVETYKKFMSLFKLMLQLRNSVTGTDIDYMISPVTDKTGTHFDSRENIKNLPADADANGAYNIARKGIMAIENIMNGISDPLKISNEDYLKYIQNQQE

SEQ ID NO: 14 - protein, 1159 aa, Leptotrichia bucallis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 15 - protein, 1182 aa, Leptotrichia wadei
MYMKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKFEILENEFLENVKAMLEYSE

SEQ ID NO: 16 - protein, 1285 aa, Herbinix hemicellulosilytics
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 17 - protein, 966 aa, Ruminococcus flavefaciens
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGN

SEQ ID NO: 18 - DNA, 2901 bp, Ruminococcus flavefaciens
atgatcgaaaaaaaaaagtccttcgccaagggcatgggcgtgaagtccacactcgtgtccggctccaaagtgtacatgacaaccttcgccgaaggcagcgacgccaggctggaaaagatcgtggagggcgacagcatcaggagcgtgaatgagggcgaggccttcagcgctgaaatggccgataaaaacgccggctataagatcggcaacgccaaattcagccatcctaagggctacgccgtggtggctaacaaccctctgtatacaggacccgtccagcaggatatgctcggcctgaaggaaactctggaaaagaggtacttcggcgagagcgctgatggcaatgacaatatttgtatccaggtgatccataacatcctggacattgaaaaaatcctcgccgaatacattaccaacgccgcctacgccgtcaacaatatctccggcctggataaggacattattggattcggcaagttctccacagtgtatacctacgacgaattcaaagaccccgagcaccatagggccgctttcaacaataacgataagctcatcaacgccatcaaggcccagtatgacgagttcgacaacttcctcgataaccccagactcggctatttcggccaggcctttttcagcaaggagggcagaaattacatcatcaattacggcaacgaatgctatgacattctggccctcctgagcggactgaggcactgggtggtccataacaacgaagaagagtccaggatctccaggacctggctctacaacctcgataagaacctcgacaacgaatacatctccaccctcaactacctctacgacaggatcaccaatgagctgaccaactccttctccaagaactccgccgccaacgtgaactatattgccgaaactctgggaatcaaccctgccgaattcgccgaacaatatttcagattcagcattatgaaagagcagaaaaacctcggattcaatatcaccaagctcagggaagtgatgctggacaggaaggatatgtccgagatcaggaaaaatcataaggtgttcgactccatcaggaccaaggtctacaccatgatggactttgtgatttataggtattacatcgaagaggatgccaaggtggctgccgccaataagtccctccccgataatgagaagtccctgagcgagaaggatatctttgtgattaacctgaggggctccttcaacgacgaccagaaggatgccctctactacgatgaagctaatagaatttggagaaagctcgaaaatatcatgcacaacatcaaggaatttaggggaaacaagacaagagagtataagaagaaggacgcccctagactgcccagaatcctgcccgctggccgtgatgtttccgccttcagcaaactcatgtatgccctgaccatgttcctggatggcaaggagatcaacgacctcctgaccaccctgattaataaattcgataacatccagagcttcctgaaggtgatgcctctcatcggagtcaacgctaagttcgtggaggaatacgcctttttcaaagactccgccaagatcgccgatgagctgaggctgatcaagtccttcgctagaatgggagaacctattgccgatgccaggagggccatgtatatcgacgccatccgtattttaggaaccaacctgtcctatgatgagctcaaggccctcgccgacaccttttccctggacgagaacggaaacaagctcaagaaaggcaagcacggcatgagaaatttcattattaataacgtgatcagcaataaaaggttccactacctgatcagatacggtgatcctgcccacctccatgagatcgccaaaaacgaggccgtggtgaagttcgtgctcggcaggatcgctgacatccagaaaaaacagggccagaacggcaagaaccagatcgacaggtactacgaaacttgtatcggaaaggataagggcaagagcgtgagcgaaaaggtggacgctctcacaaagatcatcaccggaatgaactacgaccaattcgacaagaaaaggagcgtcattgaggacaccggcagggaaaacgccgagagggagaagtttaaaaagatcatcagcctgtacctcaccgtgatctaccacatcctcaagaatattgtcaatatcaacgccaggtacgtcatcggattccattgcgtcgagcgtgatgctcaactgtacaaggagaaaggctacgacatcaatctcaagaaactggaagagaagggattcagctccgtcaccaagctctgcgctggcattgatgaaactgcccccgataagagaaaggacgtggaaaaggagatggctgaaagagccaaggagagcattgacagcctcgagagcgccaaccccaagctgtatgccaattacatcaaatacagcgacgagaagaaagccgaggagttcaccaggcagattaacagggagaaggccaaaaccgccctgaacgcctacctgaggaacaccaagtggaatgtgatcatcagggaggacctcctgagaattgacaacaagacatgtaccctgttcagaaacaaggccgtccacctggaagtggccaggtatgtccacgcctatatcaacgacattgccgaggtcaattcctacttccaactgtaccattacatcatgcagagaattatcatgaatgagaggtacgagaaaagcagcggaaaggtgtccgagtacttcgacgctgtgaatgacgagaagaagtacaacgataggctcctgaaactgctgtgtgtgcctttcggctactgtatccccaggtttaagaacctgagcatcgaggccctgttcgataggaacgaggccgccaagttcgacaaggagaaaaagaaggtgtccggcaattaa

(19) United States
(12) Patent Application Publication
(10) Pub. No.: US 2022/0365065 A1
(43) Pub. Date: Nov. 17, 2022

(54) NANOPORE-BASED DETECTION OF ANALYTES

(71) Applicant: The Penn State Research Foundation, University Park, PA (US)

(72) Inventors: Reza Nouri, State College, PA (US); Yuqian Jiang, State College, PA (US); Xiaojun Lance Lian, State College, PA (US); Weihua Guan, State College, PA (US)

(21) Appl. No.: 17/627,372

(22) PCT Filed: Jul. 17, 2020
(86) PCT No.: PCT/US2020/042616

§ 371 Date: Jan. 14, 2022

Related U.S. Application Data
(60) Provisional application No. 63/018,841, filed on May 1, 2020.
(60) Provisional application No. 62/875,235, filed on Jul. 17, 2019.

(51) Int. Cl. G01N 33/487; C12Q 1/6825

(52) U.S. Cl. CPC G01N 33/48721; C12Q 1/6825; G01N 2333/922

(57) ABSTRACT

Methods of detecting a target nucleic acid sequence analyte are provided in which a crRNA and Cas12 or Cas13 enzyme are contacted to form a non-activated RNP. The non-activated RNP is contacted with a sample containing or suspected of containing the target nucleic acid sequence, and the target nucleic acid sequence and non-activated RNP specifically bind to each other if the target nucleic acid is present in the sample, thereby forming an activated RNP. A reporter nucleic acid is contacted with the activated RNP, and the activated RNP indiscriminately cleaves the reporter nucleic acid, reducing passage of intact, non-cleaved reporter nucleic acid through a nanopore in of a nanopore counting device such that a reduction of resistive pulses is produced which provides a signal representative of presence of the target nucleic acid sequence in the sample.

### CROSS REFERENCE TO RELATED APPLICATIONS

This application claims the benefit of U.S. Provisional Application No. 62/875,235 filed Jul. 17, 2019 and 63/018,841 filed May 1, 2020, the entire contents of both of which are hereby fully incorporated herein by reference.

### GRANT REFERENCE

This invention was made with government support under Grant Nos. ECCS1710831, CBET1902503 and ECCS1912410, awarded by the National Science Foundation. The Government has certain rights in the invention.

### FIELD OF THE INVENTION

Methods and compositions according to the present disclosure relate generally to detection of analytes. According to specific aspects methods and compositions according to the present disclosure relate to detection of nucleic acid analytes using nanopore detection devices.

### BACKGROUND OF THE INVENTION

Specific and sensitive methods for detection of analytes is lacking in numerous fields. Nucleic acid detection methods are crucial for many applications, such as pathogen detection and genotyping. Many bio-sensing applications used fluorescent, bioluminescent, or colorimetric reporters for readouts, which often require optical sensing and additional design and synthesis of reporter molecules like fluorescence/quencher beacons or gold nanoparticles, implicating high cost. There is a continuing need for methods and devices for sensitive and specific detection of analytes.

### SUMMARY OF THE INVENTION

Methods of detecting an analyte in a solution are provided according to aspects of the present disclosure which include: providing a nanopore counting device comprising; a first chamber and a second chamber; a barrier having a nanopore opening defined therein, the barrier separating the first chamber from the second chamber; a control/sensing system operable to apply an electrical potential between a solution in the first chamber and a solution in the second chamber and to sense a voltage and/or current between the chambers; disposing an ion-containing solution in the first and second chambers; disposing a sample in the first chamber, the sample containing or suspected of containing the analyte; applying an electrical potential between the solution in the first chamber and the solution in the second chamber; and sensing a baseline current between the chambers and detecting resistive pulses, wherein detecting resistive pulses provides a signal representative of presence of the analyte in the sample, and wherein no calibration step is performed before disposing the sample.

Methods of detecting an analyte in a solution using a nanopore counting device are provided according to aspects of the present disclosure, wherein the nanopore counting device includes a first chamber and a second chamber; a barrier having a nanopore opening defined therein, the barrier separating the first chamber from the second chamber; and a control/sensing system operable to apply an electrical potential between a solution in the first chamber and a solution in the second chamber and to sense a voltage and/or current between the chambers; the method including disposing an ion-containing solution in the first and second chambers; disposing a sample in the first chamber, the sample containing or suspected of containing the analyte; applying an electrical potential between the solution in the first chamber and the solution in the second chamber; and sensing a baseline current between the chambers and detecting resistive pulses, wherein detecting resistive pulses provides a signal representative of presence of the analyte in the sample, and wherein no calibration step is performed before disposing the sample.

According to aspects of the present disclosure, methods of detecting an analyte in a solution are provided wherein sensing a baseline current between the chambers and detecting resistive pulses provides a count of a number of molecules of analyte that pass through the nanopore during a period of time.

According to aspects of the present disclosure, methods of detecting an analyte in a solution, further include determining an estimated concentration of the molecules of analyte in the first chamber, wherein determining comprises calculation using the formula:

$${C}_{mol} = \frac{Λ R}{μ {N}_{A} {I}_{b}} {C}_{ion}$$

where:
Λ is the molar conductivity of the buffer solution in Siemens per meter per mole;
R is the translocation rate in resistive pulses per second;
μ is the free solution electrophoretic mobility of the analyte in meters per volt second;
N_{A} is the Avogadro constant; and
I_{b} is the baseline current in nA.

According to aspects of the present disclosure, the barrier with the nanopore is a solid state nanopore barrier.

According to aspects of the present disclosure, methods of detecting an analyte in a solution are provided wherein the nanopore has an opening size larger than a size of an analyte molecule, the nanopore opening size being within one order of magnitude of the size of the analyte molecule.

According to aspects of the present disclosure, methods of detecting an analyte in a solution are provided wherein the period of time is determined by the number of resistive pulse, the number of resistive pulse during the period of time being at least 100.

According to aspects of the present disclosure, methods of detecting an analyte in a solution are provided wherein the number of resistive pulses is at least 200.

According to aspects of the present disclosure, methods of detecting an analyte in a solution are provided wherein the number of resistive pulses during the period of time is at least 200.

Methods of detecting an analyte in a solution are provided according to aspects of the present disclosure which include: providing a nanopore counting device comprising; a first chamber and a second chamber; a barrier having a nanopore opening defined therein, the barrier separating the first chamber from the second chamber; a control/sensing system operable to apply an electrical potential between a solution in the first chamber and a solution in the second chamber and to sense a voltage and/or current between the chambers; disposing an ion-containing solution in the first and second chambers; disposing a sample in the first chamber, the sample containing or suspected of containing the analyte; applying an electrical potential between the solution in the first chamber and the solution in the second chamber; and sensing a baseline current between the chambers and detecting resistive pulses, wherein detecting resistive pulses provides a signal representative of presence of the analyte in the sample, wherein no calibration step is performed before disposing the sample, and wherein the analyte is a target nucleic acid sequence.

Methods of detecting a nucleic acid analyte in a solution using a nanopore counting device are provided according to aspects of the present disclosure, wherein the nanopore counting device includes a first chamber and a second chamber; a barrier having a nanopore opening defined therein, the barrier separating the first chamber from the second chamber; and a control/sensing system operable to apply an electrical potential between a solution in the first chamber and a solution in the second chamber and to sense a voltage and/or current between the chambers; the method including disposing an ion-containing solution in the first and second chambers; disposing a sample in the first chamber, the sample containing or suspected of containing the analyte; applying an electrical potential between the solution in the first chamber and the solution in the second chamber; and sensing a baseline current between the chambers and detecting resistive pulses, wherein detecting resistive pulses provides a signal representative of presence of the analyte in the sample, and wherein no calibration step is performed before disposing the sample.

In methods of detecting a target nucleic acid sequence analyte according to aspects of the present disclosure a reporter nucleic acid is provided; a CRISPR-Cas system guide RNA (also known as a crRNA) that hybridizes to the target nucleic acid sequence is provided; and a CRISPR enzyme is provided, wherein the CRISPR enzyme is a Cas enzyme capable of forming a non-activated ribonucleoprotein (RNP) complex (non-activated RNP) with the guide RNA (also known as a crRNA), and wherein the non-activated RNP is capable of binding to the target nucleic acid sequence, thereby forming an activated RNP complex (activated RNP) having “trans” activity to indiscriminately cleave the reporter nucleic acid, wherein cleavage of the reporter nucleic acid results in at least two smaller cleaved portions of the reporter nucleic acid.

In methods of detecting a target nucleic acid sequence analyte according to aspects of the present disclosure, the crRNA (also known as a guide RNA) and Cas enzyme are contacted in a reaction vessel, thereby forming a non-activated complex of the crRNA and Cas enzyme, a non-activated RNP. The non-activated RNP is contacted with the sample containing or suspected of containing the target nucleic acid sequence in the same or in a different reaction vessel, and the target nucleic acid sequence and non-activated RNP specifically bind to each other if the target nucleic acid is present in the sample, thereby forming an activated RNP. The reporter nucleic acid is disposed in a reaction vessel with the activated RNP, wherein the activated RNP indiscriminately cleaves the reporter nucleic acid, wherein cleavage of the reporter nucleic acid results in at least two smaller cleaved portions of the reporter nucleic acid, and wherein cleavage of the reporter nucleic acid reduces passage of intact, non-cleaved reporter nucleic acid through the nanopore in the nanopore counting device such that a reduction of resistive pulses is produced which provides a signal representative of presence of the target nucleic acid sequence in the sample. A reaction vessel can be the first chamber of the nanopore counting device according to aspects of the present disclosure.

In methods of detecting a target DNA sequence analyte according to aspects of the present disclosure, the crRNA and a Cas12 enzyme are contacted in a reaction vessel, thereby forming a non-activated complex of the crRNA and Cas12 enzyme, a non-activated RNP. The non-activated RNP is contacted with the sample containing or suspected of containing the target DNA sequence in the same or in a different reaction vessel, and the target DNA sequence and non-activated RNP specifically bind to each other if the target DNA is present in the sample, thereby forming an activated RNP. The reporter nucleic acid is disposed in a reaction vessel with the activated RNP, wherein the activated RNP indiscriminately cleaves the reporter nucleic acid, wherein cleavage of the reporter nucleic acid results in at least two smaller cleaved portions of the reporter nucleic acid, and wherein cleavage of the reporter nucleic acid reduces passage of intact, non-cleaved reporter nucleic acid through the nanopore in the nanopore counting device such that a reduction of resistive pulses is produced which provides a signal representative of presence of the target DNA sequence in the sample. A reaction vessel can be the first chamber of the nanopore counting device according to aspects of the present disclosure.

In methods of detecting a target RNA sequence analyte according to aspects of the present disclosure, the crRNA and a Cas13 enzyme are contacted in a reaction vessel, thereby forming a non-activated complex of the crRNA and Cas13 enzyme, a non-activated RNP. The non-activated RNP is contacted with the sample containing or suspected of containing the target RNA sequence in the same or in a different reaction vessel, and the target DNA sequence and non-activated RNP specifically bind to each other if the target RNA is present in the sample, thereby forming an activated RNP. The reporter nucleic acid is disposed in a reaction vessel with the activated RNP, wherein the activated RNP indiscriminately cleaves the reporter nucleic acid, wherein cleavage of the reporter nucleic acid results in at least two smaller cleaved portions of the reporter nucleic acid, and wherein cleavage of the reporter nucleic acid reduces passage of intact, non-cleaved reporter nucleic acid through the nanopore in the nanopore counting device such that a reduction of resistive pulses is produced which provides a signal representative of presence of the target RNA sequence in the sample. A reaction vessel can be the first chamber of the nanopore counting device according to aspects of the present disclosure.

In methods of detecting a target nucleic acid sequence analyte according to aspects of the present disclosure, the reporter nucleic acid is a linear or circular single-stranded DNA molecule.

In methods of detecting a target nucleic acid sequence analyte according to aspects of the present disclosure, the reporter nucleic acid does not include a label, such as a fluorescent, radioactive or colored label.

In methods of detecting a target nucleic acid sequence analyte according to aspects of the present disclosure, the target nucleic acid sequence is a nucleic acid of a microorganism.

In methods of detecting a target nucleic acid sequence analyte according to aspects of the present disclosure, the target nucleic acid sequence is a nucleic acid of a virus, a bacterium, a fungus, or a parasite.

In methods of detecting a target nucleic acid sequence analyte according to aspects of the present disclosure, the sample is obtained from a mammal. In methods of detecting a target nucleic acid sequence analyte according to aspects of the present disclosure, the sample is derived from a human.

In methods of detecting a target nucleic acid sequence analyte according to aspects of the present disclosure, the sample contains one or more amplified nucleic acids. In methods of detecting a target nucleic acid sequence analyte according to aspects of the present disclosure, the sample is protein-free or substantially protein-free.

In methods of detecting a target nucleic acid sequence analyte according to aspects of the present disclosure, the sample is derived from a mammal having, or suspected of having, an infection by a virus, a bacterium, a fungus, or a parasite.

In methods of detecting a target nucleic acid sequence analyte according to aspects of the present disclosure, the sample is derived from a human having, or suspected of having, an infection by a virus, a bacterium, a fungus, or a parasite.

In methods of detecting a target nucleic acid sequence analyte according to aspects of the present disclosure, the sample is an environmental sample, containing, or suspected of containing, a virus, a bacterium, a fungus, or a parasite.

In methods of detecting a target nucleic acid sequence analyte according to aspects of the present disclosure, the target nucleic acid sequence is a nucleic acid of a human immunodeficiency virus.

In methods of detecting a target nucleic acid sequence analyte according to aspects of the present disclosure, the target nucleic acid sequence is a nucleic acid of a coronavirus.

In methods of detecting a target nucleic acid sequence analyte according to aspects of the present disclosure, the target nucleic acid sequence is a SARS-Cov-2 coronavirus nucleic acid sequence.

In methods of detecting a target nucleic acid sequence analyte according to aspects of the present disclosure, the sample is derived from a plant.

In methods of detecting a target nucleic acid sequence analyte according to aspects of the present disclosure, the sample is derived from a plant having, or suspected of having, an infection by a virus, a bacterium, a fungus, or a parasite.

Methods of detecting a target nucleic acid sequence in a solution are provided according to aspects of the present disclosure which include: providing a nanopore counting device comprising; a first chamber and a second chamber; a barrier having a nanopore opening defined therein, the barrier separating the first chamber from the second chamber; a control/sensing system operable to apply an electrical potential between a solution in the first chamber and a solution in the second chamber and to sense a voltage and/or current between the chambers; calibrating the nanopore counting device to determine a rate of translocation of molecules of a calibrant from a calibration solution through the nanopore when a calibrating electrical potential is applied between the chambers; disposing an ion-containing solution in the first and second chambers; providing a reporter nucleic acid; providing a CRISPR-Cas system guide RNA (crRNA) that specifically hybridizes to the target nucleic acid sequence; providing a CRISPR enzyme, wherein the CRISPR enzyme is a Cas enzyme capable of forming a non-activated ribonucleoprotein (RNP) complex (non-activated RNP) with the guide RNA, and wherein the non-activated RNP is capable of binding to the target nucleic acid sequence, forming an activated RNP complex (activated RNP) having “trans” activity to cleave the reporter nucleic acid; contacting the crRNA and Cas enzyme, thereby forming the non-activated RNP; disposing the non-activated RNP in the first chamber with the sample, wherein the non-activated RNP and the target nucleic acid sequence specifically bind if the target nucleic acid is present in the sample, forming an activated, wherein the activated cleaves the reporter nucleic acid; applying an electrical potential between the solution in the first chamber and the solution in the second chamber; and sensing current between the first chamber and the second chamber and detecting resistive pulses, wherein cleavage of the reporter nucleic acid reduces passage of the reporter nucleic acid through the nanopore such that a reduction of resistive pulses is produced which provides a signal representative of presence of the target nucleic acid sequence in the sample. According to aspects of the present disclosure, the barrier with the nanopore barrier is a solid state nanopore barrier or a biological nanopore barrier.

According to aspects of the present disclosure, the step of detecting resistive pulses further comprises counting resistive pulses to determine a number of reporter nucleic acid molecules that pass through the nanopore during a period of time, thereby determining a rate of translocation of the reporter nucleic acid molecules.

According to aspects of the present disclosure, methods of detecting a target nucleic acid sequence analyte further include determining the estimated concentration of the target nucleic acid sequence in the first chamber based on the reporter nucleic acid translocation rate as compared to the calibrant translocation rate.

According to aspects of the present disclosure, the calibrating step comprises: disposing an ion-containing solution in the first and second chamber and a known concentration of calibrant molecules in the first chamber, the calibrant molecules being the same or similar to the reporter nucleic acid molecules; applying the calibrating electrical potential between the chambers; sensing current between the chambers and counting resistive pulses to determine a number of molecules of the calibrant that pass through the nanopore during a period of time; and determining a rate of translocation for the known concentration of calibrant at the calibrating electrical potential.

According to aspects of the present disclosure, the calibrant molecules are the same as the reporter nucleic acid molecules, the calibrating electrical potential is in the range of 0.5 to 2 times the electrical potential used after the calibrating step, and the ion-containing solution during is the same during the calibrating step and after the calibrating step.

According to aspects of the present disclosure, the target nucleic acid sequence is DNA and the Cas enzyme is a Cas12 enzyme. According to aspects of the present disclosure, the target nucleic acid sequence is RNA and the Cas enzyme is a Cas13 enzyme. According to aspects of the present disclosure, the reporter nucleic acid is a linear circular single-stranded DNA molecule. According to aspects of the present disclosure, the reporter nucleic acid does not include a label. According to aspects of the present disclosure, the target nucleic acid sequence is a nucleic acid of a microorganism. According to aspects of the present disclosure, the target nucleic acid sequence is a nucleic acid of a virus, a bacterium, a fungus, or a parasite. According to aspects of the present disclosure, the sample is obtained from a mammal. According to aspects of the present disclosure, the sample is derived from a human. According to aspects of the present disclosure, the sample is derived from a mammal having, or suspected of having, an infection by a virus, a bacterium, a fungus, or a parasite. According to aspects of the present disclosure, the sample is derived from a human having, or suspected of having, an infection by a virus, a bacterium, a fungus, or a parasite.

According to aspects of the present disclosure, the sample is an environmental sample, containing, or suspected of containing, a virus, a bacterium, a fungus, or a parasite.

According to aspects of the present disclosure, the target nucleic acid sequence is a nucleic acid of a human immunodeficiency virus.

According to aspects of the present disclosure, the target nucleic acid sequence is a nucleic acid of a coronavirus. According to aspects of the present disclosure, the coronavirus is a SARS-Cov-2 coronavirus. According to aspects of the present disclosure, the sample is derived from a plant.

According to aspects of the present disclosure, the sample is derived from a plant having, or suspected of having, an infection by a virus, a bacterium, a fungus, or a parasite.

### BRIEF DESCRIPTION OF THE DRAWINGS

FIG. 1 is a schematic representation of a nanopore counting device which may form an aspect of the present invention;

FIGS. 2A-2D are charts illustrating current flow and the change over time as molecules translocate in an exemplary nanopore counting device;

FIG. 3A a schematic of an experimental setup;

FIG. 3B is a graph of a current-voltage (I-V) curve recorded for nanopore size estimation;

FIG. 3C is an SEM image of a glass nanopore;

FIG. 3D is a TEM image of a glass nanopore;

FIG. 4A is a series of current time traces;

FIG. 4B is a graph of extracted interarrival time distribution;

FIG. 4C is a graph showing probability distribution P(n) for observing n events within a fixed time interval;

FIG. 4D is a graph showing a curve fit for a Poisson distribution;

FIG. 5 is a graph comparing the inferred rate using the online n/T method to the rate determined by the Poisson fitting method, using the 12 pM λ-DNA sample;

FIG. 6A is a schematic showing translocation and an associated current trace;

FIG. 6B is a representation of date from experiments performed with 10 kbp DNA at 24 pM in the 1 M KCl buffer solution with two different pores and at different voltages;

FIG. 6C is a graph showing translocation rate versus baseline current;

FIG. 6D is a graph showing measured concentration versus input concentration;

FIG. 7A and FIG. 7B schematically show aspects of a Solid-State CRISPR-Cas12a- or CRISPR-Cas13-Assisted Nanopore (SCAN) sensor method of the present disclosure. FIG. 7A illustrates a positive case, in which the trans-cleavage activity of the Cas12a after activation causes degradation of the circular ssDNA reporters, resulting in reduced reporter event rate through the nanopore. FIG. 7B illustrates a negative case, the Cas12a is not activated in the absence of target dsDNA and thus the ssDNA reporters are not cleaved with the result that the nanopore event rate remains high;

FIG. 8A and FIG. 8B are images of gel electrophoresis results of buffer optimizing for an HIV-1 Cas12a assay according to aspects of the present disclosure. FIG. 8A shows results of evaluating three candidate buffers: NEBuffer 3.1, PBS buffer and IDT buffer. FIG. 8B shows results of evaluating the impact of salt concentration on Cas12a activity in the IDT buffer;

FIG. 9A and FIG. 9B are traces and a graph, respectively, showing quantitative ability at constant RNP concentrations. The traces of FIG. 9A show translocation recording of serially-diluted ssDNA reporters ranging from 50-250 pM through the glass nanopore under 400 mV bias. The RNP and buffer salt concentration were fixed as 30 nM and 1 M, respectively. The graph of FIG. 9B shows the nanopore event rate as a function of the ssDNA reporter concentrations; the error bars correspond to the Poisson noise of determining the event rate;

FIG. 10 is a graph showing the remaining ssDNA reporter concentration as a function of reaction time (0, 5, 10, 20, and 30 minutes) for different HIV target concentrations (15, 30, and 60 nM). In each case, the initial ssDNA reporter concentration was set as 100 pM and the remaining concentration was obtained using the extracted translocation rate through the nanopore. The solid line is the fitting using the Michaelis-Menten kinetics, C=C₀e^{-kT}^{ᵣ}, where C₀ is the initial ssDNA reporter concentration (100 pM). The fitted rate constants k for 15, 30, and 60 nM target HIV was obtained as 0.037, 0.051, and 0.081 min-1 respectively;

FIG. 11A is a graph illustrating distributions for event numbers observed in the negative and positive cases. The overlap of the two distributions should be less than 5% for a positive/negative call at the 95% confidence level. FIG. 11B shows the total experimental time needed for making a positive/negative call at different combinations of HIV target and RNP concentrations. The dashed dotted line indicates the region in which the qualitative call can be made within 1 hour;

FIG. 12A is an image of agarose gel electrophoresis showing results of the designed specificity test. Only specific combinations (Assay 1-Target 1, and Assay 2-Target 2) lead to the degradation of reporters. FIGS. 12B, 12C, 12D, and 12E are traces and graphs showing results of a specificity test in SCAN. The reaction time for all cases is 30 minutes and the HIV target concentration is 30 nM. Only matched Cas12a assay and its target can produce a significant reduction in the number of translocation events. The error bars correspond to the Poisson noise of determining the event rate;

FIG. 13A and FIG. 13B show a comparison between the sensitivity of traditional gel electrophoresis and a nanopore sensor in the detection of reporter residue. Reporter ssDNAs with a concentration lower than 1 nM cannot be detected by the gel images, FIG. 13A. On the other hand, the nanopore sensor shows higher sensitivity such that 100 pM of the ssDNA reporter is detected, FIG. 13B; the magnified view of a current trace illustrates a typical single molecule translocation event;

FIG. 14 is a trace showing results of a nanopore experiment for a pure RNP and HIV targets sample (30 nM) without any ssDNA reporter. No single event was observed for a measurement duration time of 1000 s, which confirms that the translocation rate of the background activated RNPs is less than 0.001 s⁻¹;

FIG. 15A is a set of scatter plots showing current dip magnitude vs. dwell times for a negative sample and positive samples (30 nM HIV-1) with different cleavage time (5, 10, 20, and 30 minutes). This data illustrates that the distribution of the current dip and dwell times do not change after cleavage. Thus, the effect of the cleaved DNAs on the nanopore sensing is negligible. FIG. 15B is a box plot of the event charge deficits (ECD) for all cases, which shows that the ECD distribution does not change significantly from the negative sample to the positive samples;

FIG. 16 is a graph showing a linear fitting of experiment results for extracting degradation rates at different RNP concentrations. The fitting slope of the fitted line is 0.00148 min-1 nM-1;

FIG. 17A and FIG. 17B show the effect of RNP concentration on the electrophoretic mobility of ssDNA reporters. FIG. 17A shows traces illustrating translocation recording of ssDNA through glass nanopore at four RNP concentrations, 0, 15, 30, and 60 nM, under 400 mV bias; these experiments were performed at 1 M salt concentration and ssDNA concentration was fixed at 100 pM. FIG. 17B is a graph showing extracted ssDNA reporter electrophoretic mobility as a function of the background RNP concentration; and

FIG. 18 is a graph showing exponential fitting of experiment results for electrophoretic mobility at different RNP concentrations.

### DETAILED DESCRIPTION

Scientific and technical terms used herein are intended to have the meanings commonly understood by those of ordinary skill in the art. Such terms are found defined and used in context in various standard references illustratively including M. Green and J. Sambrook, Molecular Cloning: A Laboratory Manual, Cold Spring Harbor Laboratory Press; 4^{th} Ed., 2012; F. M. Ausubel, Ed., Short Protocols in Molecular Biology, Current Protocols; 5th Ed., 2002; B. Alberts et al., Molecular Biology of the Cell, 4th Ed., Garland, 2002; D. Clark et al., Molecular Biology, 3^{rd} Ed., Academic Cell, 2018; CRISPR/Cas: A Laboratory Manual, Doudna and Mali (eds), Cold Spring Harbor Laboratory Press, Cold Spring Harbor, N.Y., USA, 2016; D. L. Nelson and M. M. Cox, Lehninger Principles of Biochemistry, 4th Ed., W.H. Freeman & Company, 2004; and Herdewijn, P. (Ed.), Oligonucleotide Synthesis: Methods and Applications, Methods in Molecular Biology, Humana Press, 2004.

The singular terms “a,” “an,” and “the” are not intended to be limiting and include plural referents unless explicitly stated otherwise or the context clearly indicates otherwise.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

Reference herein to “one aspect”, “an aspect,” “an example,” means that a particular feature, structure or characteristic described or named is included in at least one embodiment of the present invention. Thus, appearances of the phrases “according to aspects,” “according to an aspect,” or “an example” herein are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination

The recitation of numerical ranges by endpoints includes all numbers and fractions subsumed within the respective ranges, as well as the recited endpoints.

Nanopore Digital Counting of Single Molecules

According to an aspect of the present invention, a concentration of molecules of an analyte in a solution may be determined using a nanopore digital counting method.

According to an aspect of the present invention, nanopore devices are not modified to include specific binding sites for the analyte. Thus, the nanopore device may be used to detect any of various analytes, including nucleic acid analytes.

Nanopore-based sensors allow single molecules to be analyzed electronically without the need for labeling and partitioning, unlike with optical digital counting or bulk analog sensing. Conceptually, a nanopore sensor represents an ideal single molecule counting device due to its unique features of label-free electronic sensing, single-molecule sensitivity, and potential reusability.

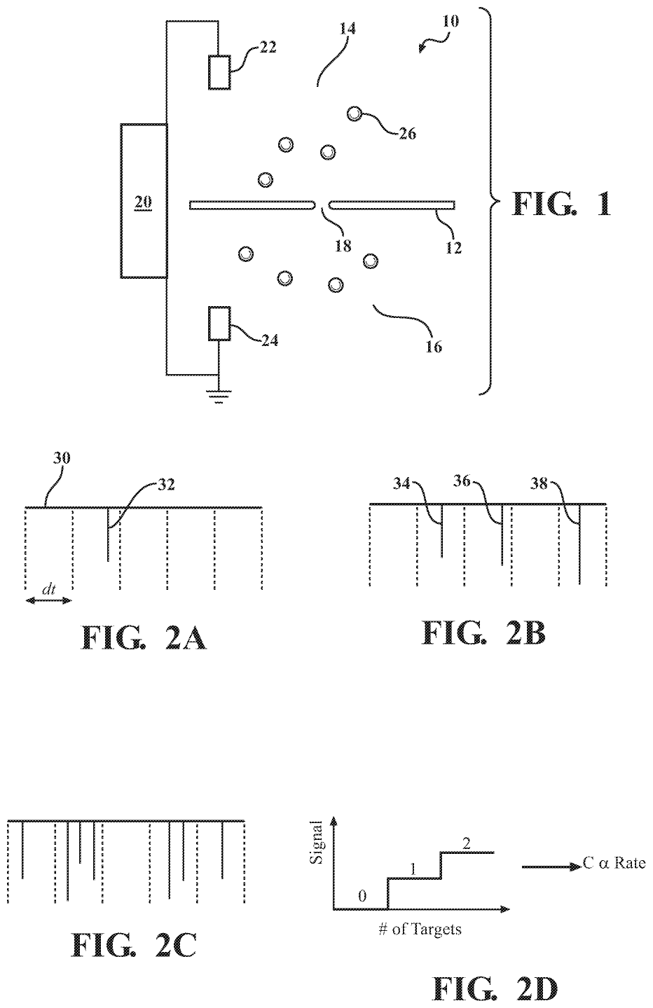

FIG. 1 provides a schematic representation of a nanopore counting device 10. A barrier 12, such as a membrane, divides a first chamber 14 from a second chamber 16. A nanopore 18 is defined in the barrier 12 to allow communication between the first chamber 14 and second chamber 16. An ion-containing solution is disposed in both chambers such that electrical current may travel through the nanopore in the presence of an electrical potential. A control/sensing system 20 is operable to provide an electrical potential between the solution in the first chamber 14 and the second chamber 16, and to sense current and/or voltage between the solutions in the chambers. As will be clear to those of skill in the art, the control/sensing system 20 may be a single combined unit or separate systems may be used to provide the electrical potential and the sense current and/or voltage. A first electrode 22 is disposed in the first chamber 14 and a second electrode 24 is disposed in the second chamber 16, with both electrodes in electrical communication with the control/sensing system.

The barrier may take a variety of forms, including biological barriers with a biological nanopore and solid state nanopores where the nanopore is created using any one of a variety of techniques.

Molecules of an analyte are represented as small spheres at 26 and are illustrated as present in both of the chambers. As will be clear to those of skill in the art, the drawing is not to scale. In one example, where the molecules of analyte 26 are DNA, the molecules have a width dimension of approximately 2 nm (nanometers) and the nanopore has a width dimension of approximately 10 nm. The nanopore is typically larger than the molecules to be analyzed, such as up to one order of magnitude larger, though other dimensions may be used.

In the presence of an electrical potential, provided by the electrodes 22 and 24, molecules of analyte translocate through the nanopore from one chamber to the other electrophoretically. When a molecule 26 is not translocating through the nanopore, current will flow through the solution at a baseline rate. When a molecule translocates, the current flow is partially obstructed causing resistance to current flow to rise and current flow to drop. FIGS. 2A-D illustrates a current flow and the change over time as molecules translocate. The solid horizontal line 30 represents time, divided into time periods dt. In FIG. 2A. a single resistive pulse is shown at 32 occurring during the second time interval. FIG. 2B shows three resistive pulses, one of which occurs in each of the second, third and fifth intervals. It is noted that pulses 34 and 36 are illustrated as smaller than pulse 38. Typically, the size of the pulse is not important to the method, as long as the pulse is large enough to indicate the passage of a molecule. In other words, the method typically does not distinguish characteristics of individual molecules based on pulse size but instead counts each pulse. This is what is meant by “digital counting.” Either a pulse occurs, which is a “yes” or a digital 1, or a pulse does not occur, which is a “no” or a digital zero. FIG. 2C illustrates an additional series of pulses. FIG. 2D shows a digital count of number of targets over time.

By counting the number of molecules that translocate through the nanopore during a period of time, a translocation rate may be determined. The translocation rate depends on the concentration of the molecules of analyte in the first chamber, the strength of the electrical potential, the size and the shape of the nanopore and various other factors. The strength of the electrical potential is controlled by the user and the size of the nanopore and most other factors are generally stable over time, so the rate of translocation therefore correlates with the analyte concentration. Put another way, a single molecule is electrophoretically driven through the nanopore, a detectable ionic current blockade generates a digital “1” signal, the rate of which is proportional to the sample concentration. Resolving this digital event itself is much easier than analyzing its analog features such as magnitude and duration of the current dip.

Existing theories and experiments have shown that when interactions between molecules are negligible, the molecule molar concentration (mol/m3) is linearly related to the translocation rate (events per second) as R=αN_{A}C, where N_{A} is the Avogadro constant and α is usually referred to as the capture rate. Since capture rate α strongly depends on experimental parameters such as nanopore geometry, temperature, molecule size, and applied voltage, it is typically necessary to define a calibration curve of the rate versus concentration to then use the nanopore digital counting method to infer the concentration in an unknown sample. Moreover, the calibration curve must be obtained under the same experimental conditions such that there is a comparable capture rate α. Unfortunately, generating this calibration curve is often time-consuming and challenging due to nanopore clogging issues.

Calibration may be performed in several ways, as long as the calibration results in determining a translocation rate versus concentration slope under conditions sufficiently similar to the conditions to be used when testing a sample with an unknown concentration. Also, the calibration should be performed with a known concentration that is not too much different from the unknown concentration. For example, if the calibration is performed with a first concentration and the unknown concentration is several orders of magnitude greater, the slope determined during the calibration step may not be applicable. This could necessitate calibration at multiple concentrations or performing a new calibration.

One approach to calibration is as follows. Starting with the same nanopore counting device to be used in the test, an ion-containing solution is disposed in the first and second chamber and a known concentration of calibrant molecules is disposed in the first chamber. As noted, the calibrant molecules need to be functionally the same or very similar to what will be used in the actual test. Typically, the same analyte molecules are used. A calibrating electrical potential is applied between the chambers to cause translocation of calibrant molecules. The calibrating electrical potential should be the same as will be used during the test. In some cases, calibration may be performed at more than one level of electrical potential so that other levels may be used during testing. In some examples, the rate determined during calibration may be applicable to potentials that are someone larger or smaller, with appropriate adjustment. For example, in some cases, the calibrating electrical potential may be in the range of 0.1 to 10 times the electrical potential used in the actual test.

Current between the chambers is then sensed and resistive pulses are digitally counted to determine a number of molecules of the calibrant that pass through the nanopore during a period of time. The interval between translocation events varies, generally indicating a Poisson process, as will be discuss later. In order to reduce uncertainty in the rate determined during calibration, the rate should be determined for a statistically significant number of translocation events. This will be further discussed later. Once enough events have been counted, a rate of translocation for the known concentration of calibrant at the calibrating electrical potential. The testing of the unknown concentration may then proceed, with the concentration being determined by determining a rate and then dividing by the slope of the rate versus concentration curve from the calibration.

Calibration-Free Nanopore Digital Counting of Single Molecules

According to a further aspect of the present invention, a method is provided for determining a concentration of molecules of an analyte in a solution using nanopore digital counting without the need for a separate calibration step prior to testing the unknown solution.

The calibration-free method makes use of a nanopore counting device that may be the same as described above with reference to FIG. 1. That is, a first chamber is separated from a second chamber by a barrier with a nanopore opening defined therein, and molecules of an analyte are electrophoretically driven through the nanopore opening. The molecules are counted during a period of time to determine a translocation rate. However, unlike with the above-described method, the calibration-free method does not require calibration prior to testing. It has been discovered that measurements of the baseline current may be used to determine characteristics of the nanopore and thereby calculate an estimated concentration.

An embodiment of the calibration-free method will be described, and then details of the theory and testing leading to the embodiment will be provided. A method of determining an estimated concentration of an analyte in a solution starts with providing a nanopore counting device. FIG. 1 provides a schematic representation of the nanopore counting device 10. A barrier 12, such as a membrane, divides a first chamber 14 from a second chamber 16. A nanopore 18 is defined in the barrier 12 to allow communication between the first chamber 14 and second chamber 16. An ion-containing solution is disposed in both chambers such that electrical current may travel through the nanopore in the presence of an electrical potential. A control/sensing system 20 is operable to provide an electrical potential between the solution in the first chamber 14 and the second chamber 16, and to sense current and/or voltage between the solutions in the chambers. As will be clear to those of skill in the art, the control/sensing system 20 may be a single combined unit or separate systems may be used to provide the electrical potential and the sense current and/or voltage. A first electrode 22 is disposed in the first chamber 14 and a second electrode 24 is disposed in the second chamber 16, with both electrodes in electrical communication with the control/sensing system.

The barrier may take a variety of forms, though typically solid state nanopores are used, where the nanopore is created using one of a variety of techniques. In an example that will be described below, a glass nanopore was used.

Molecules of an analyte are represented as small spheres at 26 and are illustrated as present in both of the chambers. As will be clear to those of skill in the art, the drawing is not to scale. In one example, where the molecules of analyte 26 are DNA, the molecules have a width dimension of approximately 2 nm (nanometers) and the nanopore has a width dimension of approximately 10 nm. The nanopore is typically larger than the molecules to be analyzed, such as up to one order of magnitude larger, though other dimensions may be used.

In the presence of an electrical potential, provided by the electrodes 22 and 24, molecules of analyte translocate through the nanopore from one chamber to the other. When a molecule 26 is not translocating through the nanopore, current will flow through the solution at a baseline rate. When a molecule translocates, the current flow is partially obstructed causing resistance to current flow to rise and current flow to drop. FIG. 2 illustrates the current flow and the change over time as molecules translocate. This step is the same as was discussed above.

By counting the number of molecules that translocate through the nanopore during a period of time, a translocation rate is determined.

Unlike the above-described method, the calibration-free method includes sensing a baseline current between the chambers in addition to counting resistive pulses to determine a number of molecules of analyte that pass through the nanopore during the period of time. The baseline current is the current when a molecule is not translocating. Put another way, the baseline current is the current between resistive pulses. As will be clear to those of skill in the art, the baseline current is typically not a constant value. Instead, the current signal typically fluctuates and may be noisy. As such, the baseline current may be an average or normalized current determined based on the fluctuating baseline current signal. The longer that baseline current is sensed, the better the average will be.

As with the earlier method, translocation events should be counted for long enough to reduce the uncertainty to an acceptable level. Once a translocation rate and a baseline current are determined, the estimated concentration of the molecules of analyte in the first chamber may be determined using the following formula:

$${C}_{mol} = \frac{Λ R}{μ {N}_{A} {I}_{b}} {C}_{ion}$$

where:
a. A is the molar conductivity of the buffer solution in Siemens per meter per mole;
b. R is the translocation rate in resistive pulses per second;
c. μ is the free solution electrophoretic mobility of the analyte in meters per volt second;
d. N_{A} is the Avogadro constant; and
e. I_{b} is the baseline current in Ampere;

As noted previously, no calibration step is performed before the step of disposing the solution with the unknown concentration in the first chamber.

The development of the calibration-free method will now be described. The inventors first studied the statistics of the molecule translocation rate and developed an experimentally practical method to measure the rate. They then developed a quantitative model for molecule quantification without the need for prior knowledge of experimental conditions such as nanopore geometry, size, and applied voltage. This was achieved by using the background ions as the in situ reference such that the molecule translocation rates were normalized to the ion translocation rates (baseline current). This model was experimentally validated for different nanopores and DNA molecules with different sizes. While the results discussed below were from glass nanopores and DNA molecules, the principle could be well extended to other nanopore types and other charged molecules.

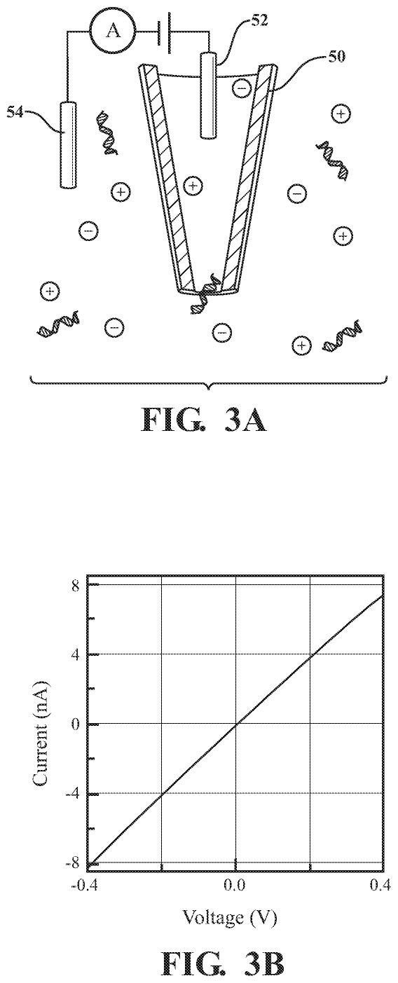

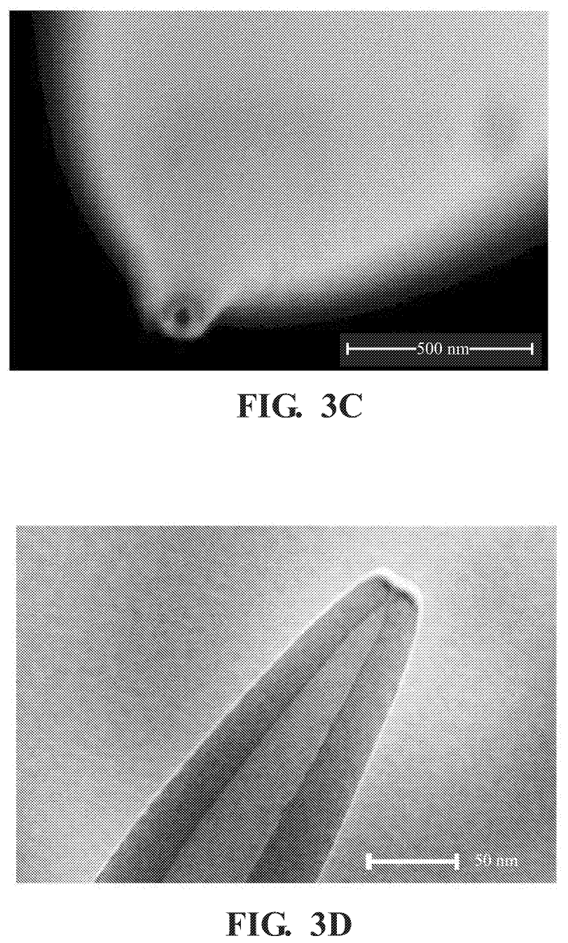

FIG. 3A provides a schematic illustration of the experimental setup. 0.2 mm Ag wires (Warner Instruments, Hamden, USA) were used to fabricate the Ag/AgCl electrodes in house. Microinjectors of 34 gauge was purchased from World Precision Instruments. Potassium chloride and Tris-EDTA-buffer solution (10 mM Tris-HCl and 1 mM EDTA) were purchased from Sigma-Aldrich. Piranha solution was made by mixing concentrated sulfuric acid (H₂SO₄) with hydrogen peroxide (H₂O₂). Quartz capillaries with inner and outer diameters of 0.5 mm and 1 mm, respectively, were purchased from Sutter Instrument. The testing solutions were filtered with a 0.2 μm syringe filter (Whatman). DNA templates (λ-DNA, 10 kbp, and 5 kbp DNA with the concentration of 0.5 μg/μL) were purchased from Thermo-Fisher. Glass nanopores were fabricated as follows. The quartz capillaries were first cleaned in Piranha solution for 30 min to remove organic residues, then rinsed with DI water, and dried in an oven at 120° C. for 15 min. A two-line recipe, (1) heat 750, filament 5, velocity 50, delay 140, and pull 50; (2) heat 710, filament 4, velocity 30, delay 155, and pull 215, were used to pull the capillaries with a laser pipet puller (P-2000, Sutter Instruments, USA). This recipe typically produces a nanopore size around 10 nm. Despite known batch-to-batch variations in size, the method presented herein is valid as long as the nanopore can resolve the single molecule translocation (rather than multiple molecules). The glass nanopore is shown at 50 in FIG. 3A, with the nanopore opening being at the bottom.

Glass nanopore characterization was performed by standard I-V (current-voltage) measurement, SEM, and TEM imaging. For I-V characterization, the glass nanopore was filled with 1 M KCl in a Tris-EDTA buffer by a microinjector and then immersed in the testing solution. Ag/AgCl electrodes 52 and 54 were used for interfacing the electrical measurement, and the I-V curve was recorded for nanopore size estimation, as shown in FIG. 3B. For SEM imaging, the glass nanopore was coated with 5 nm thick of iridium to avoid the charging effect. The SEM image is provided in FIG. 3C. TEM characterization was also performed to obtain detailed information for the nanopore geometry and size. The TEM image is provided in FIG. 3d.

The schematic of the single molecule counting setup is illustrated in FIG. 3A. One molar KCl in Tris-EDTA buffer was used for all DNA experiments to decrease the effect of electroosmotic flow. A voltage was applied across the nanopore by a 6363 DAQ card (National Instruments, USA). The resulting current was amplified by a transimpedance amplifier (DLPCA-200, FEMTO, Germany) and then digitalized by a 6363 DAQ card with a 100 kHz sampling rate. The recorded current time trace was analyzed by with customized MATLAB (MathWorks) software to extract the single molecule translocation information regarding the interarrival time between translocation events, the ionic current dip, and the molecule dwell time.

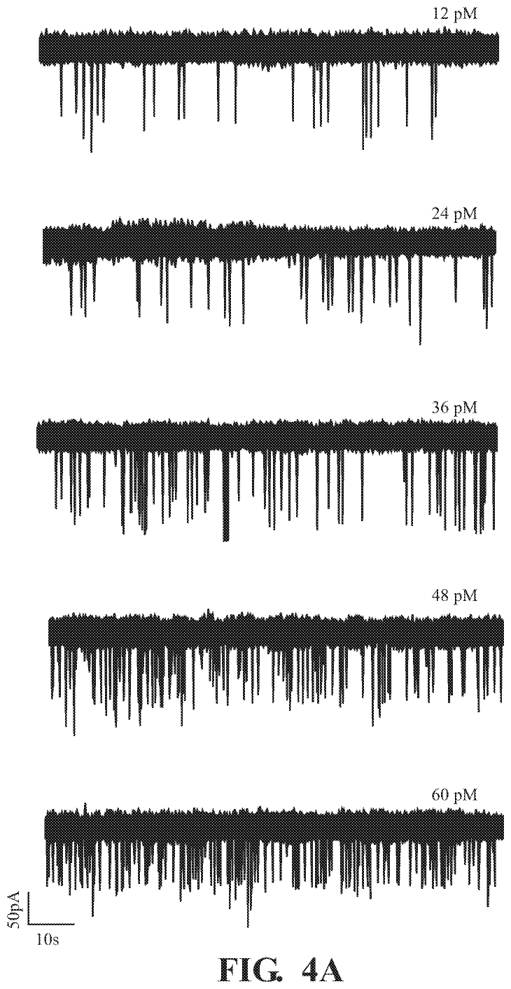

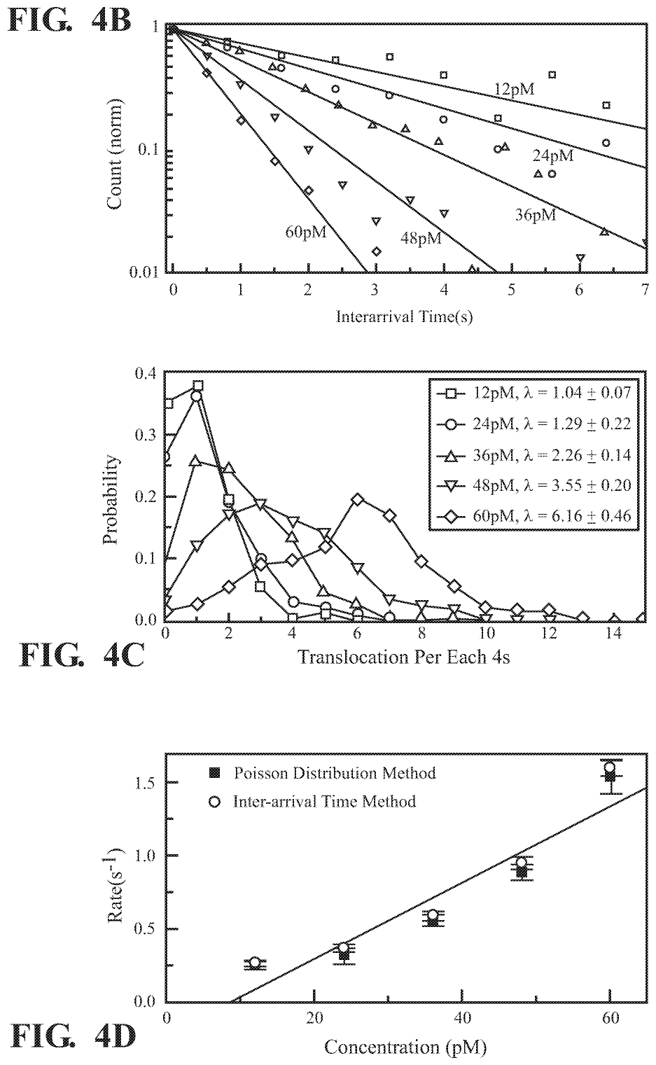

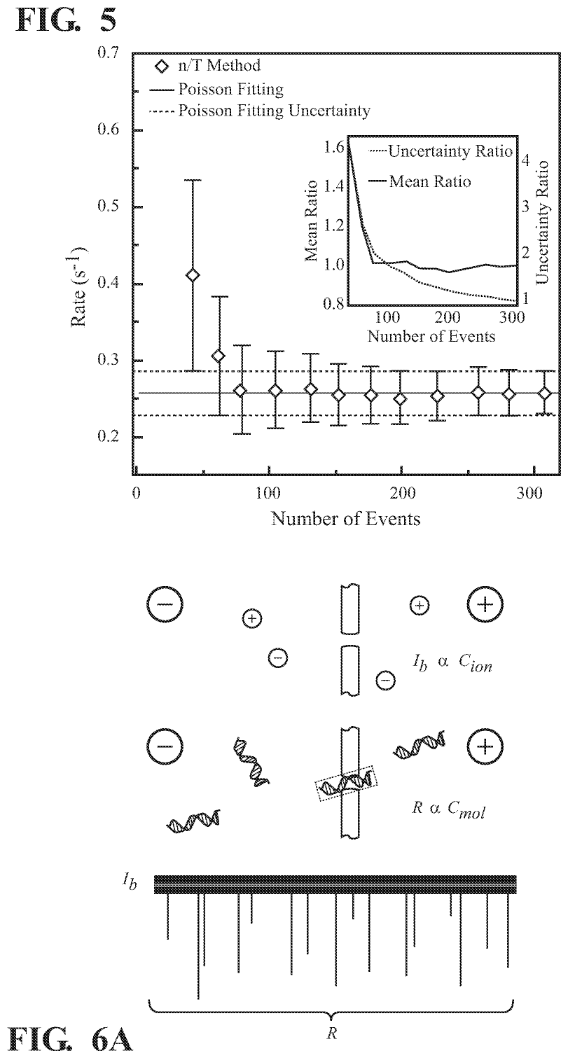

It was previously observed that the mean time between single-molecule capture events in a solid-state nanopore follows an exponential distribution, indicating a Poisson process. To validate if this was also true in the glass nanopore, studies were performed on λ-DNAs with a serial of concentrations ranging from 12 to 60 pM. A quick eyeball on the current time traces in FIG. 4A shows that translocation occurs more often as the concentration increases. The extracted interarrival time distribution also shows a remarkable exponential distribution for each concentration, as shown in FIG. 4B. The exponential fits to these distributions are usually used to obtain the hidden translocation rate. To further confirm the Poisson process, the same raw data sets were used to extract the probability distribution P(n) for observing n events within a fixed time interval, as shown in FIG. 4C. Each concentration case was then fitted with a Poisson distribution, P(n)=e^{-λ}λⁿ/n!, where λ is the expected occurrence of the events. In a process with the rate of R, λ=Rdt where dt is the time interval. As shown in FIG. 4D, both fittings to the exponentially distributed interarrival time and fittings to the Poisson distribution yield comparable rate determination at different concentrations. FIG. 4D also shows there is a linear relationship between translocation rate and the DNA concentrations in the glass nanopores, consistent with the theory prediction and previous experimental studies. While both fitting methods provide a measure of the rate R, the result can only be obtained off-line after enough digital events were registered to generate sufficient data points for fitting. A more practical approach to determine the rate online is by counting the number of events per certain time while the experiment is ongoing. Since the translocation events follow the Poisson process, assuming n discrete single-molecule translocation events were observed in a particular observation time window T, one can infer the rate with a certain confidence interval as (n±z(n)^{1/2})/T, where z is the standard score. The 95% confidence interval of the rate is (n±1.96(n)^{1/2})/T. This approach is denoted as the n/T method hereafter. The relative uncertainty of inferring the rate R is proportional to n^{-1/2}. It is thus clear that there is a trade-off between minimizing the uncertainty (increasing n) and achieving real-time rate determination (reducing n). FIG. 5 compares the inferred rate using the online n/T method to the rate determined by the Poisson fitting method, using the 12 pM λ-DNA sample. Two features were observed when the more digital translation event was observed. First, the relative uncertainty (error bars) was reduced to that of the Poisson fitting method. Second, the mean rate estimation (diamonds) converged to the trans-location rate obtained from the Poisson fitting method. These two features can be seen quantitively in the inset of FIG. 5, i.e., as more digital translations were observed, both mean and uncertainty ratios converge to 1. This validates the n/T method for rate determination as long as sufficient translocations were observed. Experimentally, the inventors examined at least 200 events for a measurement uncertainty less than 7%.

With an experimentally efficient n/T approach to determine the rate, the next task was to determine the capture rate α. The dynamics of molecule translocation through the nanopore consists of three steps: (1) the molecule moves from the bulk of the reaction chamber toward the pore entrance by a combination of diffusion and drift forces; (2) the molecule is captured at the entrance of the nanopore; and (3) the molecule overcomes an entropy energy barrier and goes through the nanopore, causing a detectable ionic current blockade which can be detected electronically as a digital signal. It is known that the capture rate α could be diffusion limited (step 1) or barrier limited (step 3). The glass nanopores used in the experiments are around 10 nm in size, which is large enough such that the transport is diffusion limited rather than barrier limited, as indicated by the linear dependence of the capture rate on the voltage.

The derivation of capture rate for the conical shaped nanopore is as follows. First, a length scale r* is introduced such that at the distances r>r* DNA is freely diffusing in the bulk solution, with potential V (r) playing a marginal role, while at r<r*, DNA gets irreversibly captured and funneled down the potential V (r*) directly to the pore mouth. If r* is estimated, then the diffusion-controlled rate is given by the classical Smoluchowski result, α=2πDr* where D is the diffusion coefficient. Assuming that the equipotential surfaces are semi-spherical outside the pore, one obtains the electrostatic potential V(r)=I/2πσr where σ is the conductivity of the nanopore and current I can be estimated by I=GΔV. For a conical nanopore, conductance can be expressed as G=σ[4l/πdₜd_{b}+1/2dₜ+1/2d_{b}]⁻¹ where l, dₜ, and d_{b} are the length and diameters of the truncated cone. Hence by defining the characteristic length d as d=1/2π[4l/πdₜd_{b}+1/2dₜ+1/2d_{b}]⁻¹, we have V(r)=ΔVd/r. Based on the previous studies, it is knows that V(r*)=D/μ, where μ is the free solution electrophoretic mobility. So r* can be estimated by r*=μd/D*ΔV. Considering the equation α=2πDr*, from above, we can derive the capture rate for a conical shape nanopore as α=2πμd ΔV.

In the diffusion-limited region, the capture rate for the conical-shaped glass nanopore is given by α=2πμdΔV, where p is the free solution electrophoretic mobility, ΔV is the applied electric potential across the pore, and d is the characteristic length of the nanopore. If the nanopore geometry and size is explicitly known for a particular experiment, the capture rate can be directly calculated to determine the unknown sample concentration without calibration, similar to a pressure-driven calibration-less quantitation of nanoparticles by calculating the hydrodynamic resistance. Nevertheless, it is well-known that glass nanopore geometry is widely dispersed. TEM characterization of each nanopore is often destructive and is time-, facility-, and expertise-intensive. In addition, experimental conditions such as applied voltage, temperatures, and buffers also vary from one experiment to the other. To properly determine the unknown sample concentration, a calibration curve must be obtained under the same experimental conditions to extract the capture rate α in that particular experiment. While this could be done, it is often time-consuming and experimentally challenging due to potential nanopore clogging under repetitive testing.

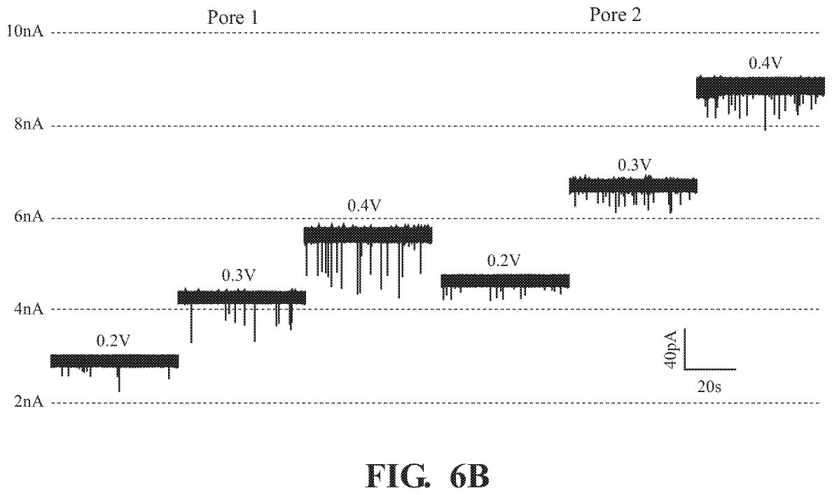

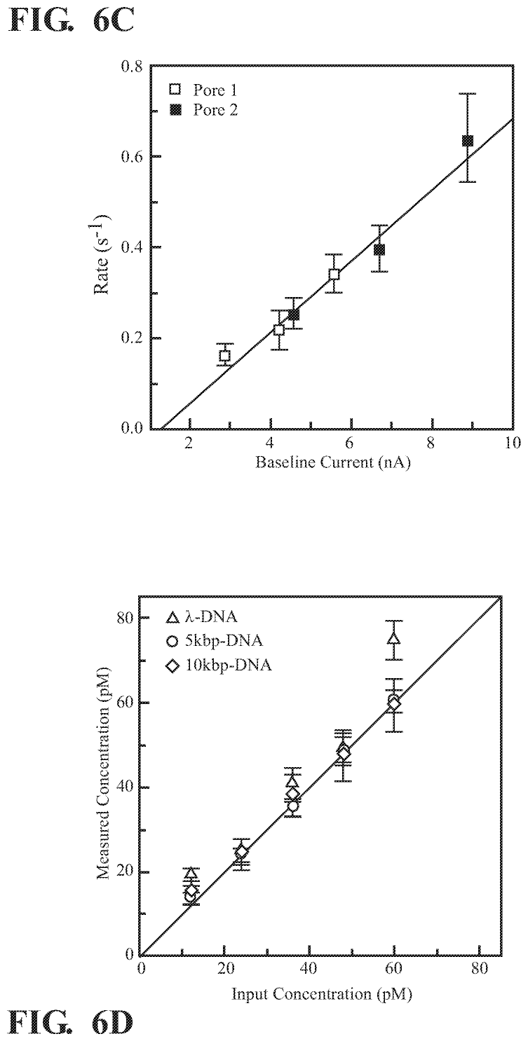

To overcome these challenges, an aspect of the present invention was developed, providing an in situ method for determining the capture rate α without the need for prior knowledge of nanopore experimental conditions. This is achieved by recognizing that the baseline current carries information about the background ion translocation rate, as shown in FIG. 6A. It was discovered that it is feasible to use the ionic concentration (generally known for a particular experiment) as the internal reference to estimate the unknown capture rate α.

The baseline current can be estimated as follows.

The baseline current is estimated based on the applied voltage ΔV and the nanopore conductance by

I_{b}=GΔV

For a conical nanopore, conductance can be expressed as

$$G = {σ [ \frac{4 l}{π {d}_{t} {d}_{b}} + \frac{1}{2 {d}_{t}} + \frac{1}{2 {d}_{b}} ]}^{- 1}$$

Similar to the last case, by defining the characteristic length

$$\tilde{d} = {\frac{1}{2 π} [ \frac{4 l}{π {d}_{t} {d}_{b}} + \frac{1}{2 {d}_{t}} + \frac{1}{2 {d}_{b}} ]}^{- 1}$$

we have:

G=2πσd

The conductivity as a function of ion species concentration and ion mobility can be written as:

$$σ = {\sum }_{i} {N}_{A} {z}_{i} e {μ}_{i} {C}_{i}$$

where N_{A} is the Avogadro constant, zᵢ is the valance, Cᵢ is the molar concentration [mole/m3], and e represents the elementary charge (1.6×10-19C), μᵢ is the ions mobility [m2/(V·s)]. Assuming monovalent ions, we can write Cᵢ=Cᵢₒₙ. As a result,

$$σ = {C}_{ion} {\sum }_{i} {eN}_{A} {z}_{i} {μ}_{i}$$

Defining the molar conductivity as

$$Λ = {\sum }_{i} {N}_{A} {ez}_{i} {μ}_{i}$$

The baseline current can thus be written and estimated as Ib=2πΛCᵢₒₙdΔV, where A is the molar conductivity which depends on the mobility and valence of the ions as Λ=ΣᵢN_{A}ezᵢμᵢ. The previously inaccessible parameter α=2πμdΔV can be rewritten as

$$α = \frac{μ {I}_{b}}{Λ {C}_{ion}}$$

This equation implies that the unknown capture rate can be derived from the experimentally accessible baseline current and the ionic concentration without knowing the nanopore geometry, size, and the applied voltage. The molecule mobility μ and molar conductivity Λ can be estimated for a particular molecule and salt. Thus, the molecule translocation rate R=αN_{A}Cₘₒₗ can be written as

$$R = \frac{μ {N}_{A} {C}_{mol}}{Λ {C}_{ion}} {I}_{b}$$

To validate this equation, experiments were performed with 10 kbp DNA at 24 pM in the 1 M KCl buffer solution. FIG. 6B shows the current time trace at different applied voltages for two glass nanopores pulled from different batches. Two features can be observed. First, higher applied voltage leads to a higher molecule translocation rate, consistent with previous reports. Second, due to the nanopore size variation, the same applied voltage does not generate the same molecule translocation rate. This dependence of the translocation rate on applied voltages and the nanopore sizes indicates that a calibration curve must be obtained under the same experimental conditions (the same pore and applied voltage). Fortunately, the molecule translocation rate equation above predicts that the molecule translocation rate scales linearly with the baseline current for a fixed testing molecule and salt concentrations. This is exactly what was observed in FIG. 6C. The molecule translocation rate versus the I_{b} indeed falls into a single line for different pores at different applied voltages. After verifying this in situ ionic current reference model, calibration-free quantification of the molecule molar concentration can thus be performed by rewriting the translocation rate equation as

$${C}_{mol} = \frac{Λ R}{μ {N}_{A} {I}_{b}} {C}_{ion}$$

This equation shows that an unknown sample concentration can be quantified without explicitly knowing the nanopore geometry, size, and the applied voltage, as long as the parameters on the right-hand side of the equation could be determined. To validate this method, tests were performed with λ-DNA, 5 kbp DNA, and 10 kbp DNA at five known concentrations (12, 24, 36, 48, and 60 pM) in 1 M KCl buffer, intentionally using glass nanopores pulled from different batches. Since the free solution electrophoretic mobility of DNA in the Tris-EDTA buffer was theoretically and experimentally shown to be independent of the DNA length longer than a few persistence lengths, μ of 4.5×10⁻⁸ m² V⁻¹ s⁻¹ was used for all DNA molecules. The buffer solution is dominated by 1 M KCl, and thus the molar conductivity A is estimated to be 10.86 m⁻¹ M⁻¹ S. Table 1 summarizes the results for this calibration-free method for concentration measurement.

TABLE 1
Summary of calibration-free method for quantifying concentration

| | Input | | | Measured | |
|---|---|---|---|---|---|
| | concentration | | | concentration | Error |
| Sample | (pM) | I_{b}(nA) | R(1/s) | (pM)* | (%)** |
| λ-DNA | 12 | 6.06 | 0.26 ± 0.03 | 18.90 ± 2.07 | 57.50 |
| | 24 | 5.93 | 0.34 ± 0.05 | 24.90 ± 3.11 | 3.75 |
| | 36 | 6.28 | 0.58 ± 0.04 | 40.78 ± 3.92 | 13.29 |
| | 48 | 8.19 | 0.92 ± 0.05 | 49.54 ± 3.54 | 3.20 |
| | 60 | 8.40 | 1.43 ± 0.11 | 74.93 ± 4.65 | 24.88 |
| 5 Kbps | 12 | 3.96 | 0.13 ± 0.01 | 13.89 ± 1.27 | 15.74 |
| DNA | 24 | 4.03 | 0.22 ± 0.01 | 24.11 ± 1.64 | 0.45 |
| | 36 | 4.05 | 0.33 ± 0.02 | 35.36 ± 2.07 | −1.77 |
| | 48 | 4.03 | 0.45 ± 0.03 | 48.70 ± 3.24 | 1.46 |
| | 60 | 4.46 | 0.61 ± 0.03 | 60.49 ± 2.71 | 0.82 |
| 10 Kbps | 12 | 6.21 | 0.21 ± 0.04 | 15.15 ± 2.87 | 26.30 |
| DNA | 24 | 6.21 | 0.34 ± 0.05 | 24.22 ± 3.63 | 0.93 |
| | 36 | 6.27 | 0.54 ± 0.07 | 38.23 ± 5.07 | 6.19 |
| | 48 | 6.83 | 0.74 ± 0.09 | 47.59 ± 6.08 | −0.85 |
| | 60 | 8.51 | 1.15 ± 0.12 | 59.53 ± 6.24 | −0.78 |

*Calculated using Eq. 3 with parameters: μ = 4.1 × 10⁻⁸ m⁻²V⁻¹s⁻¹, [symbol] = 10.86 m⁻¹M⁻¹S, Cᵢₒₙ= 1M
** Error is defined as (Measured-Input)/Input × 100%

The baseline current (Ib) and translocation rate (R) was determined from the experiment. FIG. 6d plots the measured versus the input concentration for all tests. All data points fall into a straight line of slope 1, indicating the accuracy of the calibration-free method. It is noteworthy that the molecule concentration determined by rewritten translocation rate equation is widely applicable to other kinds of molecules as long as their electrophoretic mobility was known. One important aspect of the nanopore single molecule counting method is the upper and lower bound for concentrations (dynamic range). The upper bound is related to the maximum count rate, which is determined by the speed of the electronic detector and the jamming effect when too many molecules are translocating at the same time. On the other hand, the lower bound (limit of detection) is determined by two factors. The first is the false positive rate when no molecule exists in the testing sample. This is similar to the dark count rate in the single photon counters. This false positive rate determines the minimum count rate at which the signal is dominantly caused by real molecules presented. The false detection events are mostly due to the noise in the testing apparatus. The second factor is the uncertainty in the Poisson rate determination (FIG. 5). Since relative uncertainty of inferring the rate R is proportional to n^{-1/2}, large enough event numbers (N) should be recorded to establish a sufficiently robust statistical basis. With the translocation rate R, a minimal recording time of N/R is thus required. Assuming a practical measurement time of T, a minimal translocation rate N/T is required, which corresponds to the lower bound of the molecule concentration. For example, if we need N to be 200 events and a practical experiment time of 30 min, the minimum rate should be around 0.1/s, corresponding to ˜10 pM in the above discussed experimental setup.

Nucleic Acid Assays

According to aspects of the present disclosure, the analyte is a target nucleic acid sequence. The target nucleic acid sequence is RNA or DNA.

The term “nucleic acid” as used herein refers to RNA or DNA molecules having more than one nucleotide in any form including single-stranded, double-stranded, oligonucleotide or polynucleotide. The terms “nucleotide sequence” and “nucleic acid sequence” refer to the ordering of nucleotides in an oligonucleotide or polynucleotide in reference to a single-stranded form of nucleic acid.

The term “nucleic acid” further encompasses any chemical modifications of RNA or DNA molecules, such as inclusion of one or more modified or non-naturally occurring nucleotides. Such modified or non-naturally occurring nucleotides include, but are not limited to, 2′-position sugar modifications, 5-position pyrimidine modifications, 8-position purine modifications, modifications at exocyclic amines, substitution of 4-thiouridine, substitution of 5-bromo or 5-iodo-uracil, backbone modifications, methylations, and non-standard base-pairing combinations, such as isobases, such as deoxyisocytidine and deoxyisoguanosine. Accordingly, the nucleic acids described herein include not only the standard bases adenine (A), cytosine (C), guanine (G), thymine (T), and uracil (U) but also non-standard or non-naturally occurring nucleotides.

The term “double-stranded” is used herein to refer to nucleic acids characterized by binding interaction of complementary nucleotide sequences. A double-stranded nucleic acid includes a “sense” strand and an “antisense” strand. Such duplexes include RNA/RNA, DNA/DNA or RNA/DNA types of duplexes.

The term “single-stranded” is used to refer to nucleic acids not bound by binding interaction to a complementary nucleotide sequence.

According to aspects of the present disclosure, a sample obtained from a subject or an environmental sample is assayed for a target nucleic acid according to aspects of the present disclosure.

The subject can be any organism including, a mammalian organism, a vertebrate non-mammalian organism, or a plant.

A mammalian subject can be any mammal including, but not limited to, a human; a non-human primate; a rodent such as a mouse, rat, or guinea pig; a domesticated pet such as a cat or dog; a horse, cow, pig, deer, sheep, goat, or rabbit. A non-mammalian vertebrate subject can be any vertebrate organism including, but not limited to, a bird such as a duck, goose, chicken, or turkey, a reptile, or an amphibian. Subjects can be either gender and can be any age. In aspects of methods of detecting a target nucleic acid sequence, the subject is human.

According to aspects of the present disclosure, the subject is an individual infected, or suspected of being infected, by a microorganism.

The term “sample” or “biological sample” as used herein refers to material obtained from any suitable source, including, a subject or an environment.

According to aspects of the present disclosure, a sample obtained from a subject can be any material containing, or suspected of containing, the target nucleic acid. Exemplary samples include, but are not limited to, a cell sample, a tissue sample, a fluid sample or a combination of two or more thereof. Exemplary samples include, but are not limited to, whole blood, serum, plasma, blood cells, lymph, bronchoalveolar lavage material, cerebrospinal fluid, mucus, saliva, semen, sweat, tears, amniotic fluid, wound material such as pus or a wound exudate, skin, biopsy material, synovial fluid, gastrointestinal material, vaginal fluid, fecal material, sputum, urine, any other body fluid, cell, tissue, or any material obtained from a subject that contains, or is suspected of containing a target nucleic acid, or a combination of two or more thereof.

According to aspects of the present disclosure, a sample obtained from a plant can be any portion of a plant containing, or suspected of containing, the target nucleic acid. Exemplary plant samples include, but are not limited to, a cell sample, a tissue sample, a fluid sample or a combination of two or more thereof.

Sample collection procedures for obtaining a sample from a subject are known in the art are suitable for use with various aspects of the present disclosure.

According to aspects of the present disclosure, a sample obtained from an environment can be any material containing, or suspected of containing, the target nucleic acid. Exemplary environmental samples include, but are not limited to, soil samples, air samples, water samples, aerosol samples, or a combination of any two or more thereof. An environmental sample may be, without limitation, obtained from an area, object, space, material, or a combination of any two or more thereof, such as a swab, scrape, wipe, or portion of a hospital room surface or object, clothing, furniture, equipment, food, drink, or any other object, surface, or material.

According to aspects of the present disclosure, a sample is purified to enrich for a target nucleic acid. The term “purified” in the context of a sample refers to separation of a target nucleic acid in the sample, or suspected of being present in the sample, from at least one other component present in the sample.

According to aspects of the present disclosure, a sample is purified to enrich for a target nucleic acid and remove, or substantially remove, proteins from the sample. According to aspects of the present disclosure, a sample is protein-free, or substantially protein-free, such as containing no detectable protein, less than 1 nM protein concentration, or less than 1 μM protein concentration. Detection of proteins and/or quantitation of proteins in a sample can be accomplished by any of various protein detection and/or quantitation assays, such as, but not limited to, Bradford or Lowry methods.

The term “sample” also include samples processed to enrich for nucleic acids such as by decrease or removal of other non-nucleic acid components of the sample, and/or by amplification of nucleic acids in the sample, such as by amplification of a target nucleic acid in the sample.

Amplification of a target nucleic acid is achieved using an in vitro amplification method. The term “amplification” refers to copying a target nucleic acid, thereby producing copies of the target nucleic acid.

The term “amplification” refers to methods which include template directed primer extension catalyzed by a nucleic acid polymerase using at least one primer, and in particular methods, a pair of primers which flank the target nucleic acid. Such methods include, but are not limited to, Polymerase Chain Reaction (PCR), reverse-transcription PCR (RT-PCR). ligation-mediated PCR (LM-PCR), phi-29 PCR, and other nucleic acid amplification methods, for instance, as described in C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

In particular embodiments, nucleic acids are optionally substantially purified from the sample to produce a substantially purified nucleic acid sample for use in an inventive assay. The term “substantially purified” refers to a desired material separated from other substances naturally present in a sample obtained from the subject so that the desired material makes up at least about 0.01-100% of the mass, by weight, such as about 0.01%, 0.1%, 1%, 5%, 10%, 25%, 50% 75% or greater than about 75% of the mass, by weight, of the substantially purified sample. Purification is achieved by techniques illustratively including electrophoretic methods such as gel electrophoresis and 2-D gel electrophoresis; solvent-based removal of proteins; and precipitation.

According to aspects of the present disclosure, the target nucleic acid sequence is a nucleic acid of a microorganism present in a sample obtained from a subject. The microorganism to be detected in a sample via detection of a target nucleic acid sequence of the microorganism can be any microorganism, such as, but not limited to bacteria, viruses, fungi, and parasite microorganisms, such as, but not limited to, protozoa.

Methods according to aspects of the present disclosure can be used to detect various types of bacteria, including pathogens and bacteria which are not ordinarily pathogenic (e.g. normal microflora of the gut) including, but not limited to bacteria of any of the following genera: Acidilobus, Aeropyrum, Archaeoglobus, Caldisphaera, Caldivirga, Desulfirococcus, Desulfurolobus, Ferroglobus, Ferroplasma, Geoglobus, Haloarcula, Halobacterium, Halobaculum, Halobiforma, Halococcus, Haloferax, Halogeometricum, Halomethanococcus, Halorhabdus, Halorubrobacterium, Halorubrum, Halosimplex, Haloterrigena, Hyperthermus, Ignicoccus, Metallosphaera, Methanimicrococcus, Methanobacterium, Methanobrevibacter, Methanocalculus, Methanocaldococcus, Methanococcoides, Methanococcus, Methanocorpusculum, Methanoculleus, Methanofollis, Methanogenium, Methanohalobium, Methanohalophilus, Methanolacinia, Methanolobus, Methanomicrobium, Methanomicrococcus, Methanoplanus, Methanopyrus, Methanosaeta, Methanosalsum, Methanosarcina, Methanosphaera, Methanospirillum, Methanothermobacter, Methanothermococcus, Methanothermus, Methanothrix, Methanotorris, Natrialba, Natrinema, Natronobacterium, Natronococcus, Natronomonas, Natronorubrum, Palaeococcus, Picrophilus, Pyrobaculum, Pyrococcus, Pyrodictium, Pyrolobus, Staphylothermus, Stetteria, Stygiolobus, Sulfolobus, Sulfophobococcus, Sulfurisphaera, Sulfurococcus, Thermocladium, Thermococcus, Thermodiscus, Thermofilum, Thermoplasma, Thermoproteus, Thermosphaera, and Vulcanisaeta.

Methods according to aspects of the present disclosure can be used to detect various types of bacteria, including pathogens and bacteria which are not ordinarily pathogenic (e.g. normal microflora of the gut) including, but not limited to bacteria of any of the following genera: Abiotrophia, Acetitomaculum, Acetivibrio, Acetoanaerobium, Acetobacter, Acetobacterium, Acetofilamentum, Acetogenium, Acetohalobium, Acetomicrobium, Acetonema, Acetothermus, Acholeplasma, Achromatium, Achromobacter, Acidaminobacter, Acidaminococcus, Acidimicrobium, Acidiphilium, Acidisphaera, Acidithiobacillus, Acidobacterium, Acidocella, Acidomonas, Acidothermus, Acidovorax, Acinetobacter, Acrocarpospora, Actinoalloteichus, Actinobacillus, Actinobaculum, Actinobispora, Actinocorallia, Actinokineospora, Actinomadura, Actinomyces, Actinoplanes, Actinopolymorpha, Actinopolyspora, Actinopycnidium, Actinosporangium, Actinosynnema, Aegyptianella, Aequorivita, Aerococcus, Aeromicrobium, Aeromonas, Afipia, Agitococcus, Agreia, Agrobacterium, Agrococcus, Agromonas, Agromyces, Ahrensia, Albibacter, Albidovulum, Alcaligenes, Alcalilimnicola, Alcanivorax, Algoriphagus, Alicycliphilus, Alicyclobacillus, Alishewanella, Alistipes, Alkalibacterium, Alkalilimnicola, Alkaliphilus, Alkalispirillum, Alkanindiges, Allisonella, Allochromatium, Allofustis, Alloiococcus, Allomonas, Allorhizobium, Alterococcus, Alteromonas, Alysiella, Amaricoccus, Aminobacter, Aminobacterium, Aminomonas, Ammonifex, Ammoniphilus, Amoebobacter, Amorphosporangium, Amphibacillus, Ampullariella, Amycolata, Amycolatopsis, Anaeroarcus, Anaerobacter, Anaerobaculum, Anaerobiospirillum, Anaerobranca, Anaerococcus, Anaerofilum, Anaeroglobus, Anaerolinea, Anaeromusa, Anaeromyxobacter, Anaerophaga, Anaeroplasma, Anaerorhabdus, Anaerosinus, Anaerostipes, Anaerovibrio, Anaerovorax, Anaplasma, Ancalochloris, Ancalomicrobium, Ancylobacter, Aneurinibacillus, Angiococcus, Angulomicrobium, Anoxybacillus, Anoxynatronum, Antarctobacter, Aquabacter, Aquabacterium, Aquamicrobium, Aquaspirillum, Aquifex, Arachnia, Arcanobacterium, Archangium, Arcobacter, Arenibacter, Arhodomonas, Arsenophonus, Arthrobacter, Asaia, Asanoa, Asteroleplasma, Asticcacaulis, Atopobacter, Atopobium, Aurantimonas, Aureobacterium, Azoarcus, Azomonas, Azomonotrichon, Azonexus, Azorhizobium, Azorhizophilus, Azospira, Azospirillum, Azotobacter, Azovibrio, Bacillus, Bacterionema, Bacteriovorax, Bacteroides, Bactoderma, Balnearium, Balneatrix, Bartonella, Bdellovibrio, Beggiatoa, Beijerinckia, Beneckea, Bergeyella, Beutenbergia, Bifidobacterium, Bilophila, Blastobacter, Blastochloris, Blastococcus, Blastomonas, Blattabacterium, Bogoriella, Bordetella, Borrelia, Bosea, Brachybacterium, Brachymonas, Brachyspira, Brackiella, Bradyrhizobium, Branhamella, Brenneria, Brevibacillus, Brevibacterium, Brevinema, Brevundimonas, Brochothrix, Brucella, Brumimicrobium, Buchnera, Budvicia, Bulleidia, Burkholderia, Buttiauxella, Butyrivibrio, Caedibacter, Caenibacterium, Calderobacterium, Caldicellulosiruptor, Caldilinea, Caldimonas, Caldithrix, Caloramator, Caloranaerobacter, Calymmatobacterium, Caminibacter, Caminicella, Campylobacter, Capnocytophaga, Capsularis, Carbophilus, Carboxydibrachium, Carboxydobrachium, Carboxydocella, Carboxydothermus, Cardiobacterium, Camimonas, Carnobacterium, Caryophanon, Caseobacter, Catellatospora, Catenibacterium, Catenococcus, Catenuloplanes, Catonella, Caulobacter, Cedecea, Cellulomonas, Cellulophaga, Cellulosimicrobium, Cellvibrio, Centipeda, Cetobacterium, Chainia, Chelatobacter, Chelatococcus, Chitinophaga, Chlamydia, Chlamydophila, Chlorobaculum, Chlorobium, Chloroflexus, Chloroherpeton, Chloronema, Chondromyces, Chromatium, Chromobacterium, Chromohalobacter, Chryseobacterium, Chryseomonas, Chrysiogenes, Citricoccus, Citrobacter, Clavibacter, Clevelandina, Clostridium, Cobetia, Coenonia, Collinsella, Colwellia, Comamonas, Conexibacter, Conglomeromonas, Coprobacillus, Coprococcus, Coprothermobacter, Coriobacterium, Corynebacterium, Couchioplanes, Cowdria, Coxiella, Craurococcus, Crenothrix, Crinalium (not validly published), Cristispira, Croceibacter, Crocinitomix, Crossiella, Cryobacterium, Cryomorpha, Cryptobacterium, Cryptosporangium, Cupriavidus, Curtobacterium, Cyclobacterium, Cycloclasticus, Cystobacter, Cytophaga, Dactylosporangium, Dechloromonas, Dechlorosoma, Deferribacter, Defluvibacter, Dehalobacter, Dehalospirillum, Deinobacter, Deinococcus, Deleya, Delftia, Demetria, Dendrosporobacter, Denitrobacterium, Denitrovibrio, Dermabacter, Dermacoccus, Dermatophilus, Derxia, Desemzia, Desulfacinum, Desulfitobacterium, Desulfobacca, Desulfobacter, Desulfobacterium, Desulfobacula, Desulfobulbus, Desulfocapsa, Desulfocella, Desulfococcus, Desulfofaba, Desulfofrigus, Desulfofustis, Desulfohalobium, Desulfomicrobium, Desulfomonas, Desulfomonile, Desulfomusa, Desulfonatronovibrio, Desulfonatronum, Desulfonauticus, Desulfonema, Desulfonispora, Desulforegula, Desulforhabdus, Desulforhopalus, Desulfosarcina, Desulfospira, Desulfosporosinus, Desulfotalea, Desulfotignum, Desulfotomaculum, Desulfovibrio, Desulfovirga, Desulfurella, Desulfurobacterium, Desulfuromonas, Desulfuromusa, Dethiosulfovibrio, Devosia, Dialister, Diaphorobacter, Dichelobacter, Dichotomicrobium, Dictyoglomus, Dietzia, Diplocalyx, Dolosicoccus, Dolosigranulum, Dorea, Duganella, Dyadobacter, Dysgonomonas, Ectothiorhodospira, Edwardsiella, Eggerthella, Ehrlichia, Eikenella, Elytrosporangium, Empedobacter, Enhydrobacter, Enhygromyxa, Ensifer, Enterobacter, Enterococcus, Enterovibrio, Entomoplasma, Eperythrozoon, Eremococcus, Erwinia, Erysipelothrix, Erythrobacter, Erythromicrobium, Erythromonas, Escherichia, Eubacterium, Ewingella, Excellospora, Exiguobacterium, Facklamia, Faecalibacterium, Faenia, Falcivibrio, Ferribacterium, Ferrimonas, Fervidobacterium, Fibrobacter, Filibacter, Filifactor, Filobacillus, Filomicrobium, Finegoldia, Flammeovirga, Flavimonas, Flavobacterium, Flectobacillus, Flexibacter, Flexistipes, Flexithrix, Fluoribacter, Formivibrio, Francisella, Frankia, Frateuria, Friedmanniella, Frigoribacterium, Fulvimarina, Fulvimonas, Fundibacter, Fusibacter, Fusobacterium, Gallibacterium, Gallicola, Gallionella, Garciella, Gardnerella, Gelidibacter, Gelria, Gemella, Gemmata, Gemmatimonas, Gemmiger, Gemmobacter, Geobacillus, Geobacter, Geodermatophilus, Georgenia, Geothrix, Geotoga, Geovibrio, Glaciecola, Globicatella, Gluconacetobacter, Gluconoacetobacter, Gluconobacter, Glycomyces, Gordonia, Gordonia, Gracilibacillus, Grahamella, Granulicatella, Grimontia, Haemobartonella, Haemophilus, Hafnia, Hahella, Halanaerobacter, Halanaerobium, Haliangium, Haliscomenobacter, Hallella, Haloanaerobacter, Haloanaerobium, Halobacillus, Halobacteroides, Halocella, Halochromatium, Haloincola, Halomicrobium, Halomonas, Halonatronum, Halorhodospira, Halospirulina, Halothermothrix, Halothiobacillus, Halovibrio, Helcococcus, Heliobacillus, Helicobacter, Heliobacterium, Heliophilum, Heliorestis, Heliothrix, Herbaspirillum, Herbidospora, Herpetosiphon, Hippea, Hirschia, Histophilus, Holdemania, Hollandina, Holophaga, Holospora, Hongia, Hydrogenobacter, Hydrogenobaculum, Hydrogenophaga, Hydrogenophilus, Hydrogenothermus, Hydrogenovibrio, Hymenobacter, Hyphomicrobium, Hyphomonas, Ideonella, Idiomarina, Ignavigranum, Ilyobacter, Inquilinus, Intrasporangium, Iodobacter, Isobaculum, Isochromatium, Isosphaera, Janibacter, Jannaschia, Janthinobacterium, Jeotgalibacillus, Jeotgalicoccus, Johnsonella, Jonesia, Kerstersia, Ketogulonicigenium, Ketogulonigenium, Kibdelosporangium, Kineococcus, Kineosphaera, Kineosporia, Kingella, Kitasatoa, Kitasatospora, Kitasatosporia, Klebsiella, Kluyvera, Knoellia, Kocuria, Koserella, Kozakia, Kribbella, Kurthia, Kutzneria, Kytococcus, Labrys, Lachnobacterium, Lachnospira, Lactobacillus, Lactococcus, Lactosphaera, Lamprobacter, Lamprocystis, Lampropedia, Laribacter, Lautropia, Lawsonia, Lechevalieria, Leclercia, Legionella, Leifsonia, Leisingera, Leminorella, Lentibacillus, Lentzea, Leptonema, Leptospira, Leptospirillum, Leptothrix, Leptotrichia, Leucobacter, Leuconostoc, Leucothrix, Levinea, Lewinella, Limnobacter, Limnothrix, Listeria, Listonella, Lonepinella, Longispora, Lucibacterium, Luteimonas, Luteococcus, Lysobacter, Lyticum, Macrococcus, Macromonas, Magnetospirillum, Malonomonas, Mannheimia, Maricaulis, Marichromatium, Marinibacillus, Marinilabilia, Marinilactibacillus, Marinithermus, Marinitoga, Marinobacter, Marinobacterium, Marinococcus, Marinomonas, Marinospirillum, Marmoricola, Massilia, Megamonas, Megasphaera, Meiothermus, Melissococcus, Melittangium, Meniscus, Mesonia, Mesophilobacter, Mesoplasma, Mesorhizobium, Methylarcula, Methylobacillus, Methylobacter, Methylobacterium, Methylocaldum, Methylocapsa, Methylocella, Methylococcus, Methylocystis, Methylomicrobium, Methylomonas, Methylophaga, Methylophilus, Methylopila, Methylorhabdus, Methylosarcina, Methylosinus, Methylosphaera, Methylovorus, Micavibrio, Microbacterium, Microbispora, Microbulbifer, Micrococcus, Microcyclus, Microcystis, Microellobosporia, Microlunatus, Micromonas, Micromonospora, Micropolyspora, Micropruina, Microscilla, Microsphaera, Microtetraspora, Microvirga, Microvirgula, Mitsuokella, Mobiluncus, Modestobacter, Moellerella, Mogibacterium, Moorella, Moraxella, Morganella, Moritella, Morococcus, Muricauda, Muricoccus, Mycetocola, Mycobacterium, Mycoplana, Mycoplasma, Myroides, Myxococcus, Nannocystis, Natroniella, Natronincola, Natronoincola, Nautilia, Neisseria, Neochlamydia, Neorickettsia, Neptunomonas, Nesterenkonia, Nevskia, Nitrobacter, Nitrococcus, Nitrosococcus, Nitrosolobus, Nitrosomonas, Nitrosospira, Nitrospina, Nitrospira, Nocardia, Nocardioides, Nocardiopsis, Nonomuraea, Nonomuria, Novosphingobium, Obesumbacterium, Oceanicaulis, Oceanimonas, Oceanisphaera, Oceanithermus, Oceanobacillus, Oceanobacter, Oceanomonas, Oceanospirillum, Ochrobactrum, Octadecabacter, Oenococcus, Oerskovia, Okibacterium, Oleiphilus, Oleispira, Oligella, Oligotropha, Olsenella, Opitutus, Orenia, Oribaculum, Orientia, Ornithinicoccus, Ornithinimicrobium, Ornithobacterium, Oscillochloris, Oscillospira, Oxalicibacterium, Oxalobacter, Oxalophagus, Oxobacter, Paenibacillus, Pandoraea, Pannonibacter, Pantoea, Papillibacter, Parachlamydia, Paracoccus, Paracraurococcus, Paralactobacillus, Paraliobacillus, Parascardovia, Parvularcula, Pasteurella, Pasteuria, Paucimonas, Pectinatus, Pectobacterium, Pediococcus, Pedobacter, Pedomicrobium, Pelczaria, Pelistega, Pelobacter, Pelodictyon, Pelospora, Pelotomaculum, Peptococcus, Peptoniphilus, Peptostreptococcus, Persephonella, Persicobacter, Petrotoga, Pfennigia, Phaeospirillum, Phascolarctobacterium, Phenylobacterium, Phocoenobacter, Photobacterium, Photorhabdus, Phyllobacterium, Pigmentiphaga, Pilimelia, Pillotina, Pimelobacter, Pirella, Pirellula, Piscirickettsia, Planctomyces, Planktothricoides, Planktothrix, Planobispora, Planococcus, Planomicrobium, Planomonospora, Planopolyspora, Planotetraspora, Plantibacter, Pleisomonas, Plesiocystis, Plesiomonas, Polaribacter, Polaromonas, Polyangium, Polynucleobacter, Porphyrobacter, Porphyromonas, Pragia, Prauserella, Prevotella, Prochlorococcus, Prochloron, Prochlorothrix, Prolinoborus, Promicromonospora, Propionibacter, Propionibacterium, Propionicimonas, Propioniferax, Propionigenium, Propionimicrobium, Propionispira, Propionispora, Propionivibrio, Prosthecobacter, Prosthecochloris, Prosthecomicrobium, Proteus, Protomonas, Providencia, Pseudaminobacter, Pseudoalteromonas, Pseudoamycolata, Pseudobutyrivibrio, Pseudocaedibacter, Pseudomonas, Pseudonocardia, Pseudoramibacter, Pseudorhodobacter, Pseudospirillum, Pseudoxanthomonas, Psychrobacter, Psychroflexus, Psychromonas, Psychroserpens, Quadricoccus, Quinella, Rahnella, Ralstonia, Ramlibacter, Raoultella, Rarobacter, Rathayibacter, Reichenbachia, Renibacterium, Rhabdochromatium, Rheinheimera, Rhizobacter, Rhizobium, Rhizomonas, Rhodanobacter, Rhodobaca, Rhodobacter, Rhodobium, Rhodoblastus, Rhodocista, Rhodococcus, Rhodocyclus, Rhodoferax, Rhodoglobus, Rhodomicrobium, Rhodopila, Rhodoplanes, Rhodopseudomonas, Rhodospira, Rhodospirillum, Rhodothalassium, Rhodothermus, Rhodovibrio, Rhodovulum, Rickettsia, Rickettsiella, Riemerella, Rikenella, Rochalimaea, Roseateles, Roseburia, Roseibium, Roseiflexus, Roseinatronobacter, Roseivivax, Roseobacter, Roseococcus, Roseomonas, Roseospira, Roseospirillum, Roseovarius, Rothia, Rubrimonas, Rubritepida, Rubrivivax, Rubrobacter, Ruegeria, Rugamonas, Ruminobacter, Ruminococcus, Runella, Saccharobacter, Saccharococcus, Saccharomonospora, Saccharopolyspora, Saccharospirillum, Saccharothrix, Sagittula, Salana, Salegentibacter, Salibacillus, Salinibacter, Salinibacterium, Salinicoccus, Salinisphaera, Salinivibrio, Salmonella, Samsonia, Sandaracinobacter, Sanguibacter, Saprospira, Sarcina, Sarcobium, Scardovia, Schineria, Schlegelella, Schwartzia, Sebaldella, Sedimentibacter, Selenihalanaerobacter, Selenomonas, Seliberia, Serpens, Serpula, Serpulina, Serratia, Shewanella, Shigella, Shuttleworthia, Silicibacter, Simkania, Simonsiella, Sinorhizobium, Skermanella, Skermania, Slackia, Smithella, Sneathia, Sodalis, Soehngenia, Solirubrobacter, Solobacterium, Sphaerobacter, Sphaerotilus, Sphingobacterium, Sphingobium, Sphingomonas, Sphingopyxis, Spirilliplanes, Spirillospora, Spirillum, Spirochaeta, Spiroplasma, Spirosoma, Sporanaerobacter, Sporichthya, Sporobacter, Sporobacterium, Sporocytophaga, Sporohalobacter, Sporolactobacillus, Sporomusa, Sporosarcina, Sporotomaculum, Staleya, Staphylococcus, Stappia, Starkeya, Stella, Stenotrophomonas, Sterolibacterium, Stibiobacter, Stigmatella, Stomatococcus, Streptacidiphilus, Streptimonospora, Streptoalloteichus, Streptobacillus, Streptococcus, Streptomonospora, Streptomyces: S. abikoensis, S. erumpens, S. erythraeus, S. michiganensis, S. microflavus, S. zaomyceticus, Streptosporangium, Streptoverticillium, Subtercola, Succiniclasticum, Succinimonas, Succinispira, Succinivibrio, Sulfitobacter, Sulfobacillus, Sulfurihydrogenibium, Sulfurimonas, Sulfurospirillum, Sutterella, Suttonella, Symbiobacterium, Symbiotes, Synergistes, Syntrophobacter, Syntrophobotulus, Syntrophococcus, Syntrophomonas, Syntrophospora, Syntrophothermus, Syntrophus, Tannerella, Tatlockia, Tatumella, Taylorella, Tectibacter, Teichococcus, Telluria, Tenacibaculum, Tepidibacter, Tepidimonas, Tepidiphilus, Terasakiella, Teredinibacter, Terrabacter, Terracoccus, Tessaracoccus, Tetragenococcus, Tetrasphaera, Thalassomonas, Thalassospira, Thauera, Thermacetogenium, Thermaerobacter, Thermanaeromonas, Thermanaerovibrio, Thermicanus, Thermithiobacillus, Thermoactinomyces, Thermoanaerobacter, Thermoanaerobacterium, Thermoanaerobium, Thermobacillus, Thermobacteroides, Thermobifida, Thermobispora, Thermobrachium, Thermochromatium, Thermocrinis, Thermocrispum, Thermodesulfobacterium, Thermodesulforhabdus, Thermodesulfovibrio, Thermohalobacter, Thermohydrogenium, Thermoleophilum, Thermomicrobium, Thermomonas, Thermomonospora, Thermonema, Thermosipho, Thermosyntropha, Thermoterrabacterium, Thermothrix, Thermotoga, Thermovenabulum, Thermovibrio, Thermus, Thialkalicoccus, Thialkalimicrobium, Thialkalivibrio, Thioalkalicoccus, Thioalkalimicrobium, Thioalkalispira, Thioalkalivibrio, Thiobaca, Thiobacillus, Thiobacterium, Thiocapsa, Thiococcus, Thiocystis, Thiodictyon, Thioflavicoccus, Thiohalocapsa, Thiolamprovum, Thiomargarita, Thiomicrospira, Thiomonas, Thiopedia, Thioploca, Thiorhodococcus, Thiorhodospira, Thiorhodovibrio, Thiosphaera, Thiospira, Thiospirillum, Thiothrix, Thiovulum, Tindallia, Tissierella, Tistrella, Tolumonas, Toxothrix, Trabulsiella, Treponema, Trichlorobacter, Trichococcus, Tropheryma, Tsukamurella, Turicella, Turicibacter, Tychonema, Ureaplasma, Ureibacillus, Vagococcus, Vampirovibrio, Varibaculum, Variovorax, Veillonella, Verrucomicrobium, Verrucosispora, Vibrio, Victivallis, Virgibacillus, Virgisporangium, Virgosporangium, Vitellibacter, Vitreoscilla, Vogesella, Volcaniella, Vulcanithermus, Waddlia, Weeksella, Weissella, Wigglesworthia, Williamsia, Wolbachia, Wolinella, Xanthobacter, Xanthomonas, Xenophilus, Xenorhabdus, Xylanimonas, Xylella, Xylophilus, Yersinia, Yokenella, Zavarzinia, Zobellia, Zoogloea, Zooshikella, Zymobacter, Zymomonas, and Zymophilus.

Methods according to aspects of the present disclosure can be used to detect various types of virus including, but not limited to dsDNA viruses (e.g. Adenoviruses, Herpesviruses, Poxviruses); ssDNA viruses (+ strand or “sense”) DNA (e.g. Parvoviruses); dsRNA viruses (e.g. Reoviruses); (+)ssRNA viruses (+ strand or sense) RNA (e.g. Coronaviruses, Picornaviruses, Togaviruses); (−)ssRNA viruses (− strand or antisense) RNA (e.g. Orthomyxoviruses, Rhabdoviruses); ssRNA-RT viruses (+ strand or sense) RNA with DNA intermediate in life-cycle (e.g. Retroviruses); and dsDNA-RT viruses DNA with RNA intermediate in life-cycle (e.g. Hepadnaviruses).

Methods according to aspects of the present disclosure can be used to detect various types of virus, including pathogens and viruses which are not ordinarily pathogenic (e.g. viral vectors for nucleic acid delivery) including, but not limited to viruses of any of the following families: Anelloviridae, Arenaviridae, Arterivirus, Astroviridae, Birnaviridae, Bromoviridae, Bunyaviridae, Caliciviridae, Closteroviridae, Comoviridae, Coronaviridae, Cystoviridae, Filoviridae, Flaviviridae, Flexiviridae, Hepadnaviridae, Hepevirus, Herpesviridae, Leviviridae, Luteoviridae, Mesoniviridae, Mononegavirales, Mosaic Viruses, Nidovirales, Nodaviridae, Orthomyxoviridae, Papillomaviridae, Paramyxoviridae, Parvoviridae, Picobirnaviridae, Picornaviridae, Polyomaviridae, Potyviridae, Poxviridae, Pneumoviridae, Reoviridae, Retroviridae, Rhabdoviridae, Roniviridae, Sequiviridae, Tenuivirus, Togaviridae, Tombusviridae, Totiviridae, and Tymoviridae.

Methods according to aspects of the present disclosure can be used to detect various types of viral infection caused by one or more of: adeno-associated virus, adenovirus, Aichi virus, Alfuy virus, Australian bat lyssavirus, Banna virus, Banzi virus, Barmah forest virus, BK polyomavirus, bovine diarrhea virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, cytomegalovirus, Dengue virus (DNV), Dhori virus, Dugbe virus, Duvenhage virus, Eastern equine encephalitis virus, Ebolavirus, Echovirus, Encephalomyocarditis virus (EMCV), Enterovirus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Human herpesvirus 1, Human herpesvirus 2, Human herpesvirus 6, Human herpesvirus 7, Human herpesvirus 8, human cytomegalovirus (hCMV), Human immunodeficiency virus, Horsepox virus, Ilheus virus, influenza virus, including avian influenza virus, human influenza virus, and swine influenza virus, Influenza A virus, Influenza B virus, Influenza C virus, human papillomavirus 1, human papillomavirus 2, human papillomavirus 16, human papillomavirus 18, Human parainfluenza, Human parvovirus B19, Human spumaretrovirus, Human T-lymphotropic virus, Human torovirus, Isfahan virus, Japanese encephalitis virus, JC polyomavirus, Junin virus, KI Polyomavirus, Kokobera virus, Kunjin virus, Kyasanur forest disease virus, Lagos bat virus, Lake Victoria Marburgvirus, Langat virus, Lassa virus, Lordsdale virus, louping-ill virus, Lymphocytic choriomeningitis virus, Machupo virus, Marburg virus, Mayaro virus, measles virus, Mengo encephalomyocarditis virus, Merkel cell polyomavirus, MERS-coronavirus (MERS), metapneumovirus, Molluscum contagiosum virus, Mokola virus, Monkeypox virus, Mosaic Viruses, Mumps virus, Murray Valley encephalitis virus, New York virus, Nipah virus, norovirus, O'nyong-nyong virus, Orf virus, Oropouche virus, parainfluenza virus, Pichinde virus, poliovirus, Powassan virus, Punta toro phlebovirus, Puumala virus, Rabies virus, respiratory syncytial virus (RSV), rhinovirus, Rift valley fever virus, Rocio virus, Rosavirus A, Ross river virus, Rotavirus A, Rotavirus B, RotavirusC, Rubella virus, Sagiyama virus, Salivirus A, Sandfly fever sicilian virus, Sapporo virus, SARS-coronavirus (SARS), SARS-CoV-2 coronavirus, Semliki forest virus, Seoul virus, Simian foamy virus, Simian virus 5, Sindbis virus, Southampton virus, St. Louis encephalitis virus, Tacaribe virus, Tick-borne powassan virus, tick-borne encephalitis virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, yellow fever virus, and Zika virus.

Methods according to aspects of the present disclosure can be used to detect various types of fungal or yeast infection caused by an organism such as, without limitation: Aspergillus, Blastomyces, Candida, Candida auris, Coccidioides, Cryptococcus neoformans, Cryptococcus gatti, Histoplasma, mucormycetes, Pneumocystis jirovecii, Sporothrix, Epidermophyton floccosum, fungi of the genus Trichophyton including Trichophyton rubrum and Trichophyton mentagrophytes, Trichophyton mengninii, Trichophyton schoenleinii, Trichophyton tonsurans, Micosporum canis, Microsporum audouinii, Microsporum gypseum, and Pityrosporum orbicular

Fungal diseases include aspergillosis, blastomycosis, candiasis, coccidiomycosis, cryptococcosis, histoplasmosis, mucorycosis, mycetoma, pneumocystis pneumonia, dermatophytosis, sporotrichosis, paracoccidioidmycosis, pseudallescheriasis, and talaromycosis.

Methods according to aspects of the present disclosure can be used to detect various types of infection caused by a parasitic organism such as, without limitation: protozoans including Sarcodina, Mastigophora, Ciliophora, and Sporozoa; helminths including platyhelminths, acanthocephalins, and nematodes; and ectoparasites, including ticks, fleas, lice, and mites.

Methods according to aspects of the present disclosure can be used to detect various types of infection caused by a parasitic organism such as, without limitation: Cryptosporidium parvum, Cryptosporidium hominis, Cyclospora cayetanensis, Entamoeba histolytica, Giardia lamblia, Plasmodium falciparum, Plasmodium vivax, Plasmodium malariae, Plasmodium ovale, and Plasmodium knowlesi.

Parasitic diseases include, without limitation, malaria, giardiasis, Babesiosis, cyclosporiasis, cryptosporidiosis, amoebiasis lymphatic filariasis, Leishmaniasis, onchocerciasis, schistosomiasis, Toxoplasmosis, trichomoniasis, trypanosomiasis, and Guinea worm disease, and organisms associated with these or other parasitic diseases can be assayed according to aspects of the present disclosure

A reporter nucleic acid is provided according to aspects of the present disclosure which is capable of being cleaved by “trans” cleavage, also called “collateral” cleavage and “off-target” cleavage of an activated ribonucleoprotein (RNP) complex of a Cas12 or Cas 13 protein and a guide RNA (crRNA) specifically bound to a target nucleic acid, wherein specific binding of the crRNA and the target nucleic acid includes specific binding of complementary nucleic acid sequences.

The term “complementary” as used herein refers to Watson-Crick base pairing between nucleotides and specifically refers to nucleotides hydrogen-bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” or “percent homology” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′; and the nucleotide sequence 3′-TCGA-5′ is 100% complementary to a region of the nucleotide sequence 5′-TAGCTG-3′.

A crRNA oligonucleotide that is specific for a target nucleic acid will specifically hybridize to the target nucleic acid under suitable conditions. As used herein, the terms “hybridize,” “hybridization,” “hybridizing,” or grammatical equivalents thereof refer to the process by which an oligonucleotide single strand anneals with a complementary strand through base pairing under defined hybridization conditions. The terms “specific hybridization,” “specifically hybridize,” “specifically hybridized,” and the like, indicate that two nucleic acid sequences share a high degree of complementarity. Specific hybridization complexes form under permissive annealing conditions and remain hybridized after subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm, for example, nearest-neighbor parameters, and conditions for nucleic acid hybridization are known in the art.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002. High stringency hybridization conditions are those which only allow hybridization of substantially complementary nucleic acids. Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

A reporter nucleic acid sequence can be DNA or RNA depending on the Cas protein used. The reporter nucleic acid sequence can be any size or conformation detectable by the nanopore counting device of the present disclosure and capable of being cleaved by the “trans” cleavage activity of the Cas protein used. Typically, the reporter nucleic acid sequence has a size in the range of about 500 nucleotides to about 100,000 nucleotides, such as in the range of about 750 nucleotides to about 50,000 nucleotides, such as in the range of about 900 nucleotides to about 25,000 nucleotides, such as in the range of about 1,000 nucleotides to about 10,000 nucleotides.

A reporter nucleic acid sequence provided according to aspects of the present disclosure is a single-stranded DNA molecule or a single-stranded RNA molecule. The reporter nucleic acid sequence may be linear or circular.

A reporter nucleic acid provided according to aspects of the present disclosure does not include an exogenous label such as a fluorescent label, a chemiluminescent label, a bioluminescent label, a magnetic particle, a radioisotope, or a chromophore.

A target nucleic acid sequence is a nucleic acid sequence of interest and the object of analysis in an assay method according to the present disclosure. The target nucleic acid can be RNA or DNA, depending on the Cas protein used. The target nucleic acid can be genomic DNA of an organism present in, or suspected of being present in, a sample. The target nucleic acid can be RNA transcribed from DNA of an organism present in, or suspected of being present in, a sample. The target nucleic acid can be viral DNA or RNA of a virus present in, or suspected of being present in, a sample. The target nucleic acid can be bacterial DNA or RNA of bacteria present in, or suspected of being present in, a sample. The target nucleic acid can be fungal DNA or RNA of a fungus present in, or suspected of being present in, a sample. The target nucleic acid can be parasite DNA or RNA of a parasite present in, or suspected of being present in, a sample. A target nucleic acid sequence may be selected which is specific for a particular organism to be detected, such as, a species or strain of virus or a species or strain of bacteria. A target nucleic acid sequence may be selected which is common to a number of organisms of a similar type, such as, a genus of virus or a genus of bacteria.

According to aspects, a protospacer adjacent motif (PAM) or PAM-like motif is adjacent the target nucleic acid sequence which directs binding of the non-activated RNP complex to the target nucleic acid to form an activated RNP complex.

The PAM may be a 5′ PAM located upstream of the 5′ end of the target nucleic acid sequence (also known as a protospacer), or a 3′ PAM located downstream of the 5′ end of the target nucleic acid. PAMs are typically 2-5 base pair sequences adjacent the target nucleic acid sequence. For Cas12a, a prototypical PAM is TTTV, where V is A, C, or G, although different Cas 12 enzymes prefer or require particular PAMs. Particular PAMs for particular Cas12 enzymes are known in the art or can be determined using known methodology, see for example, T. Jacobsen et al., Nucleic Acids Research, 48(10): 5624-5638, 2020. Cas13 enzymes typically do not require a PAM.

A CRISPR-Cas system guide RNA (crRNA) is provided according to aspects of the present disclosure that specifically hybridizes to the target nucleic acid sequence. The crRNA contains a target-specific nucleotide sequence (also called a “spacer”) complementary, or substantially complementary, to the target nucleic acid or a region of the target nucleic acid. The target-specific nucleotide sequence of the crRNA contains about 15 to 50 nucleotides, preferably 20 to 25 nucleotides. The target-specific nucleotide sequence of the crRNA contains about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 nucleotides, or preferably 20, 21, 22, 23, 24, or 25 nucleotides.

According to aspects of the present disclosure, the target-specific nucleotide sequence of the crRNA is substantially complementary to the target sequence, having at least 50%, 55%, 60%, 65%, 70%, 75%, 80%, 85%, 90%, 95%, 96%, 97%, 98%, 99% or more complementarity to the target nucleic acid sequence. According to aspects of the present disclosure, there are one or more differences between the target-specific nucleotide sequence of the crRNA and the target nucleic acid sequence, such as a one base pair mismatch between the target-specific nucleotide sequence of the crRNA and the target nucleic acid sequence, such as a two base pair mismatch between the target-specific nucleotide sequence of the crRNA and the target nucleic acid sequence, such as a three base pair mismatch between the target-specific nucleotide sequence of the crRNA and the target nucleic acid sequence, such as a four base pair mismatch between the target-specific nucleotide sequence of the crRNA and the target sequence, or such as a five base pair mismatch between the target-specific nucleotide sequence of the crRNA and the target nucleic acid sequence.

The ability of a crRNA to form a complex with a Cas12 or Cas13 enzyme and form an activated complex with a target nucleic acid sequence may be assessed by any suitable assay. For example, the crRNA, Cas enzyme, and target nucleic acid may be included in a reaction vessel under reaction conditions, followed by an assessment of preferential targeting, such cleavage of the target nucleic acid sequence, assessed by any suitable assay, such as, but not limited to gel electrophoresis. Other assessment methods are possible, and will be recognized by those skilled in the art.

The crRNA further contains a direct repeat (DR) sequence located 5′ (i.e. upstream) or 3′ (i.e. downstream) of the target-specific nucleotide sequence which interacts with the Cas12 or Cas13 protein. Typically a DR sequence is about 18 to 40 nucleotides in length which may form a hairpin stem-loop structure.

A crRNA may be produced by expression using recombinant molecular biology techniques. For example, a crRNA may be designed as a DNA molecule which can be translated to RNA using an in vitro transcription/expression system. A crRNA may also be obtained commercially.

The terms “expression,” “expressing,”. “expresses” and grammatical equivalents refer to transcription of a gene to produce a corresponding mRNA and/or translation of the mRNA to produce the corresponding protein.

A nucleic acid encoding one or more nucleic acid sequences, such as a crRNA, can be cloned into an expression vector for expression of the encoded nucleic acid sequence.

A nucleic acid encoding one or more peptides or proteins, such as a Cas protein, can be cloned into an expression vector for expression of the encoded peptides and/or protein(s).

The term “expression vector” is used to refer to a double-stranded recombinant nucleotide sequence containing a desired coding sequence and containing one or more regulatory elements necessary or desirable for the expression of the operably-linked coding sequence.

Expression vectors can be prokaryotic vectors, e.g., plasmids, or shuttle vectors, insect vectors, or eukaryotic vectors, and expression can be in vitro or in vivo. Suitable expression systems, and associated expression methods, are well known in the art and described, e.g., in Sambrook et al., Molecular Cloning, A Laboratory Manual (2nd ed. 1989; 3rd ed., 2001); Kriegler, Gene Transfer and Expression: A Laboratory Manual (1990); and Ausubel et al., Current Protocols in Molecular Biology. Such nucleic acids and proteins may also be chemically synthesized by well-known methods or may be obtained from commercial sources. Further, kits including suitable expression systems are commercially available.

An included Cas enzyme has “trans” activity to cleave the reporter nucleic acid at least once, thereby changing the size and/or conformation of the reporter nucleic acid.

According to aspects of the present disclosure, a CRISPR/Cas protein is used in methods according to aspects of the present disclosure, wherein the CRISPR/Cas protein includes a Cas protein capable of forming a non-activated ribonucleoprotein (RNP) complex (non-activated RNP) with the guide RNA (crRNA), and wherein the non-activated RNP is capable of binding to the target nucleic acid, thereby forming an activated RNP complex (activated RNP) having “trans” activity to cleave the reporter nucleic acid at least once, thereby changing the size and/or conformation of the reporter nucleic acid, thereby producing a detectable signal indicative of the presence of the target nucleic acid in the sample.

CRISPR/Cas proteins characterized by capability to indiscriminately cleave non-target nucleic acids once “activated” by formation of an activated RNP complex include Type V and Type VI CRISPR/Cas systems, including Cas12 including subtypes V-A, V-B, and variants thereof, and Cas13 including subtypes VI-A, VI-B, VI-C, VI-D (also known as Cas13a, Cas13b, Cas13c, and Cas13d) and variants thereof. Cas13a is also known as C2c2.

CRISPR/Cas12 proteins target single stranded DNA and, once an activated complex is formed including a Cas12 protein, crRNA, and the target RNA, Cas12 proteins indiscriminately cleave non-target single-stranded DNA.

Non-limiting examples of Cas12 proteins used in methods according to aspects of the present disclosure are those from an organism selected from the group consisting of: Acidaminococcus sp., Bacteroidales, Clostridia, Fibrobacteraceae, Lachnospiraceae, Prevotella sp., Spirochaetia, Succinivibrionaceae,

Non-limiting examples of Cas12 proteins used in methods according to aspects of the present disclosure are those from an organism selected from the group consisting of: Acidaminococcus, Agathobacter, Anaerovibrio, Arcobacter, Bacteroides, Butyrivibrio, Flavobacterium, and Treponema Campylobacter, Corynebacter, Eubacterium, Fiihfactor, Flaviivola, Flavobacterium, Francisella, Helcococcus, Oribacterium, Pseudobutyrivibrio, Proteocatella, Sneathia, Sulfuricurvum, Synergistes, and Treponema.

Cas12 proteins have been isolated and characterized from all of the above and include, but are not limited to: Arcobacter butzleri L348 (AbCas12a), Agathobacter rectalis strain 2789STDY5834884 (ArCas12a), Acidaminococcus sp. BV3L6 (AsCas12a), Anaerovibrio sp. RM50 (As2Cas12a), Bacteroidales bacterium KA00251 (BbCas12a), Bacteroidetes oral taxon 274 (BoCas12a), Butyrivibrio sp. NC3005 (BsCas12a), Candidate division WS6 bacterium (C6Cas12a), Coprococcus eutactus, Treponema endosymbiont of Eucomonympha sp. (EsCas12a), Fibrobacter succinogenes, Flavobacterium branchiophilum FL-15 (FbCas12a), Francisella novicida (FnCas12a), Helcococcus kunzii ATCC 51366 (HkCas12a), Lachnospira pectinoschiza strain 2789STDY5834886 (LpCas12a), Lachnospiraceae bacterium (LbCas12a), Candidatus Methanomethylophilus alvus Mx1201 (MaCas12a), Oribacterium sp. NK2B42 (OsCas12a), Candidatus Peregrinibacteria bacterium GW2011 (PbCas12a), Parcubacteria group bacterium GW2011 (PgbCas12a), Proteocatella sphenisci DSM 23131 (PsCas12a), Pseudobutyrivibrio ruminis CF1b (PrCas12a), Pseudobutyrivibrio xylanivorans strain DSM 10317 (PxCas12a), Candidatus Roizmanbacteria bacterium GW2011 (RbCas12a), Sneathia amnii strain SN3 (SaCas12a), Sulfuricurvum sp. PC08-66 (SsCas12a), Synergistes jonesii strain 78-1 (SjCas12a), Succinivibrio dextrinosolvens H5 (SdCas12a), Thiomicrospira sp. XS5 (TsCas12a), and Uncultured bacterium (gcode 4) ACD 3C00058 (U4Cas12a), any of which may be used in methods according to aspects of the present disclosure.

A non-limiting example of a Cas12a protein
is Francisella tularensis subsp. novicida U112
(FnCas12a), 1300 aa:
(SEQ ID NO: 11)
MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRNN
A non-limiting example of a Cas12 protein is
Corynebacterium diphtheria Cas12, 933 aa:
(SEQ ID NO: 12)
MSVVDQADQWRRARSVQAYSLWAKSGSEDLYLRLPQHLIDAACVAEWLWN
NWVSDSLKSTLSAAWRLPAEEVGRLYTFYAGTHDVGKATISFQRLVEKTS
HGNYLLGPVREAGLSLQWTLNEGEGKKFPHGMASALIIAAWLEKHDIDPS
SAARLSFIADAHHGFASDEELYRSHEDTLDYYPPEWLVVHAEILDSMAEI
TDIGETLEELADQSTPSAPAMQIMTGLVIMADWIASDEKAFPYVCDSSQH
DRVVEGMSHVNLPPAWVPTDVPDNVETLFRDTFTWPDSYQVRPVQRAAVA
VARAVQDPTLIIIEAPTGEGKTEAGLATSHILGQKTGAQGIFFAAPTMST
ANGLFERTKNWAQCTSSRGEVASLYLAHSKNKLSLPFQSLRFTSIGEDDH
LEKHGSVVASQWLSGRHRGILSDFVVGTVDQVLMMALQVRFSMLRHVGLA
GKIVIIDEVHAYDAYMSQYLYLTLQWLAKYGVSVILMSATLPPQQRARLV
NAYASQVCKKADASALNSDAYPLITAVNKKGISVTEVPQENSDTTIKIRR
IDDSLPALGGMFSDLLVDGGIALVICNTIRRAQQAYDSLKAIFPDEVELH
HAAFIATQRSEKEDALRESLGPHASRGEDRPWRRIVVATQVAEQSLDIDA
DVLVTDIAPIDLIIQRAGRLHRHERPHSDRPEILGQPQIFIRGINNEEIS
GELPEFDGGAAAIYGEKILYATVAYLPDEFHRPSDVPKLVKNVYSDTPCI
PENWKEQWEQACVKAKENYEKSVRKAQTFSFPQPHMARTLRDLFKQQHSN
SVDKKEESGSAQVRDAEFSIEVVALLKTEYGYHPFGREEEIENGRELTWK
EAEALAGNTVRLPAQMTRRDSDFNAVIDSLEAQTPPEWQRSGLLKGQVAL
FFDERGEARLGRFLVRYTNERGLEVEVCPKEDA
A non-limiting example of a Cas12 protein is
Proteocatella sphenisci Cas12a, 1154 aa:
(SEQ ID NO: 13)
MENFKNLYPINKTLRFELRPYGKTLENFKKSGLLEKDAFKANSRRSMQAI
IDEKFKETIEERLKYTEFSECDLGNMTSKDKKITDKAATNLKKQVILSFD
DEIFNNYLKPDKNIDALFKNDPSNPVISTFKGFTTYFVNFFEIRKHIFKG
ESSGSMAYRIIDENLTTYLNNIEKIKKLPEELKSQLEGIDQIDKLNNYNE
FITQSGITHYNEIIGGISKSENVKIQGINEGINLYCQKNKVKLPRLTPLY
KMILSDRVSNSFVLDTIENDTELIEMISDLINKTEISQDVIMSDIQNIFI
KYKQLGNLPGISYSSIVNAICSDYDNNFGDGKRKKSYENDRKKHLETNVY
SINYISELLTDTDVSSNIKMRYKELEQNYQVCKENFNATNWMNIKNIKQS
EKTNLIKDLLDILKSIQRFYDLFDIVDEDKNPSAEFYTWLSKNAEKLDFE
FNSVYNKSRNYLTRKQYSDKKIKLNFDSPTLAKGWDANKEIDNSTIIMRK
FNNDRGDYDYFLGIWNKSTPANEKIIPLEDNGLFEKMQYKLYPDPSKMLP
KQFLSKIWKAKHPTTPEFDKKYKEGRHKKGPDFEKEFLHELIDCFKHGLV
NHDEKYQDVFGFNLRNTEDYNSYTEFLEDVERCNYNLSFNKIADTSNLIN
DGKLYVFQIWSKDFSIDSKGTKNLNTIYFESLFSEENMIEKMFKLSGEAE
IFYRPASLNYCEDIIKKGHHHAELKDKFDYPIIKDKRYSQDKFFFHVPMV
INYKSEKLNSKSLNNRTNENLGQFTHIIGIDRGERHLIYLTVVDVSTGEI
VEQKHLDEIINTDTKGVEHKTHYLNKLEEKSKTRDNERKSWEAIETIKEL
KEGYISHVINEIQKLQEKYNALIVMENLNYGFKNSRIKVEKQVYQKFETA
LIKKFNYIIDKKDPETYIHGYQLTNPITTLDKIGNQSGIVLYIPAWNTSK
IDPVTGFVNLLYADDLKYKNQEQAKSFIQKIDNIYFENGEFKFDIDFSKW
NNRYSISKTKWTLTSYGTRIQTFRNPQKNNKWDSAEYDLTEEFKLILNID
GTLKSQDVETYKKFMSLFKLMLQLRNSVTGTDIDYMISPVTDKTGTHFDS
RENIKNLPADADANGAYNIARKGIMAIENIMNGISDPLKISNEDYLKYIQ
NQQE

CRISPR/Cas13 proteins target single-stranded RNA and, once an activated complex is formed including a Cas13 protein, crRNA, and the target RNA, Cas13 proteins indiscriminately cleave non-target single-stranded RNA.

Non-limiting examples of Cas13 proteins used in methods according to aspects of the present disclosure are those from an organism selected from the group consisting of: Azospirillum, Bacteroides, Campylobacter, Corynebacter, Eubacterium, Filifactor, Flaviivola, Flavobacterium, Gluconacetobacter, Lactobacillus, Legionella, Leptotrichia, Listeria, Mycoplasma, Neisseria, Nitratifractor, Parvibaculum, Roseburia, Sphaerochaeta, Staphylococcus, Streptococcus, Sutterella, and Treponema.

Cas13 proteins have been isolated and characterized from the following: Leptotrichia buccalis, Leptotrichia shahii, Leptotrichia wadei, Ruminococcus flavefaciens, Bergeyella zoohelcum, Prevotella buccae, Eubacteriaceae bacterium, Eubacterium rectale, Listeria seeligeri, Carnobacterium gallinarum, Clostridium aminophilum, Herbinix hemicellulosilytics, Lachnospiraceae bacterium, Leptotrichia buccalis, Listeria weihenstephanensis, Listeriaceae bacterium, Paludibacter propionicigenes, Rhodobacter capsulatus, see for example Abudayyeh, Omar O., et al., Nature, 550(7675), p. 280, 2017, any of which may be used in methods according to aspects of the present disclosure.

A non-limiting example of a Cas13 protein
is Leptotrichia buccalis, CRISPR-associated
endoribonuclease Cas13a, 1159 aa:
(SEQ ID NO: 14)
MKVTKVGGISHKKYTSEGRLVKSESEENRTDERLSALLNMRLDMYIKNPS
STETKENQKRIGKLKKFFSNKMVYLKDNTLSLKNGKKENIDREYSETDIL
ESDVRDKKNFAVLKKIYLNENVNSEELEVFRNDIKKKLNKINSLKYSFEK
NKANYQKINENNIEKVEGKSKRNIIYDYYRESAKRDAYVSNVKEAFDKLY
KEEDIAKLVLEIENLTKLEKYKIREFYHEIIGRKNDKENFAKIIYEEIQN
VNNMKELIEKVPDMSELKKSQVFYKYYLDKEELNDKNIKYAFCHFVEIEM
SQLLKNYVYKRLSNISNDKIKRIFEYQNLKKLIENKLLNKLDTYVRNCGK
YNYYLQDGEIATSDFIARNRQNEAFLRNIIGVSSVAYFSLRNILETENEN
DITGRMRGKTVKNNKGEEKYVSGEVDKIYNENKKNEVKENLKMFYSYDFN
MDNKNEIEDFFANIDEAISSIRHGIVHFNLELEGKDIFAFKNIAPSEISK
KMFQNEINEKKLKLKIFRQLNSANVFRYLEKYKILNYLKRTRFEFVNKNI
PFVPSFTKLYSRIDDLKNSLGIYWKTPKTNDDNKTKEIIDAQIYLLKNIY
YGEFLNYFMSNNGNFFEISKEIIELNKNDKRNLKTGFYKLQKFEDIQEKI
PKEYLANIQSLYMINAGNQDEEEKDTYIDFIQKIFLKGFMTYLANNGRLS
LIYIGSDEETNTSLAEKKQEFDKFLKKYEQNNNIKIPYEINEFLREIKLG
NILKYTERLNMFYLILKLLNHKELTNLKGSLEKYQSANKEEAFSDQLELI
NLLNLDNNRVTEDFELEADEIGKFLDFNGNKVKDNKELKKFDTNKIYFDG
ENIIKHRAFYNIKKYGMLNLLEKIADKAGYKISIEELKKYSNKKNEIEKN
HKMQENLHRKYARPRKDEKFTDEDYESYKQAIENIEEYTHLKNKVEFNEL
NLLQGLLLRILHRLVGYTSIWERDLRFRLKGEFPENQYIEEIFNFENKKN
VKYKGGQIVEKYIKFYKELHQNDEVKINKYSSANIKVLKQEKKDLYIRNY
IAHFNYIPHAEISLLEVLENLRKLLSYDRKLKNAVMKSVVDILKEYGFVA
TFKIGADKKIGIQTLESEKIVHLKNLKKKKLMTDRNSEELCKLVKIMFEY
KMEEKKSEN
A non-limiting example of a Cas13 protein is
Leptotrichia wadei (strain F0279, LwaCas13a)
CRISPR-associated endoribonuclease Cas13a,
1182 aa:
(SEQ ID NO: 15)
MYMKITKIDGVSHYKKQDKGILKKKWKDLDERKQREKIEARYNKQIESKI
YKEFFRLKNKKRIEKEEDQNIKSLYFFIKELYLNEKNEEWELKNINLEIL
DDKERVIKGYKFKEDVYFFKEGYKEYYLRILFNNLIEKVQNENREKVRKN
KEFLDLKEIFKKYKNRKIDLLLKSINNNKINLEYKKENVNEEIYGINPTN
DREMTFYELLKEIIEKKDEQKSILEEKLDNFDITNFLENIEKIFNEETEI
NIIKGKVLNELREYIKEKEENNSDNKLKQIYNLELKKYIENNFSYKKQKS
KSKNGKNDYLYLNFLKKIMFIEEVDEKKEINKEKFKNKINSNFKNLFVQH
ILDYGKLLYYKENDEYIKNTGQLETKDLEYIKTKETLIRKMAVLVSFAAN
SYYNLFGRVSGDILGTEVVKSSKTNVIKVGSHIFKEKMLNYFFDFEIFDA
NKIVEILESISYSIYNVRNGVGHFNKLILGKYKKKDINTNKRIEEDLNNN
EEIKGYFIKKRGEIERKVKEKFLSNNLQYYYSKEKIENYFEVYEFEILKR
KIPFAPNFKRIIKKGEDLFNNKNNKKYEYFKNFDKNSAEEKKEFLKTRNF
LLKELYYNNFYKEFLSKKEEFEKIVLEVKEEKKSRGNINNKKSGVSFQSI
DDYDTKINISDYIASIHKKEMERVEKYNEEKQKDTAKYIRDFVEEIFLTG
FINYLEKDKRLHFLKEEFSILCNNNNNVVDFNININEEKIKEFLKENDSK
TLNLYLFFNMIDSKRISEFRNELVKYKQFTKKRLDEEKEFLGIKIELYET
LIEFVILTREKLDTKKSEEIDAWLVDKLYVKDSNEYKEYEEILKLFVDEK
ILSSKEAPYYATDNKTPILLSNFEKTRKYGTQSFLSEIQSNYKYSKVEKE
NIEDYNKKEEIEQKKKSNIEKLQDLKVELHKKWEQNKITEKEIEKYNNTT
RKINEYNYLKNKEELQNVYLLHEMLSDLLARNVAFFNKWERDFKFIVIAI
KQFLRENDKEKVNEFLNPPDNSKGKKVYFSVSKYKNTVENIDGIHKNFMN
LIFLNNKFMNRKIDKMNCAIWVYFRNYIAHFLHLHTKNEKISLISQMNLL
IKLFSYDKKVQNHILKSTKTLLEKYNIQINFEISNDKNEVFKYKIKNRLY
SKKGKMLGKNNKFEILENEFLENVKAMLEYSE
A non-limiting example of a Cas13 protein is
Herbinix hemicellulosilytics HheC2c2, 1285 aa:
(SEQ ID NO: 16)
MKLTRRRISGNSVDQKITAAFYRDMSQGLLYYDSEDNDCTDKVIESMDFE
RSWRGRILKNGEDDKNPFYMFVKGLVGSNDKIVCEPIDVDSDPDNLDILI
NKNLTGFGRNLKAPDSNDTLENLIRKIQAGIPEEEVLPELKKIKEMIQKD
IVNRKEQLLKSIKNNRIPFSLEGSKLVPSTKKMKWLFKLIDVPNKTFNEK
MLEKYWEIYDYDKLKANITNRLDKTDKKARSISRAVSEELREYHKNLRTN
YNRFVSGDRPAAGLDNGGSAKYNPDKEEFLLFLKEVEQYFKKYFPVKSKH
SNKSKDKSLVDKYKNYCSYKVVKKEVNRSIINQLVAGLIQQGKLLYYFYY
NDTWQEDFLNSYGLSYIQVEEAFKKSVMTSLSWGINRLTSFFIDDSNTVK
FDDITTKKAKEAIESNYFNKLRTCSRMQDHFKEKLAFFYPVYVKDKKDRP
DDDIENLIVLVKNAIESVSYLRNRTFHFKESSLLELLKELDDKNSGQNKI
DYSVAAEFIKRDIENLYDVFREQIRSLGIAEYYKADMISDCFKTCGLEFA
LYSPKNSLMPAFKNVYKRGANLNKAYIRDKGPKETGDQGQNSYKALEEYR
ELTWYIEVKNNDQSYNAYKNLLQLIYYHAFLPEVRENEALITDFINRTKE
WNRKETEERLNTKNNKKHKNFDENDDITVNTYRYESIPDYQGESLDDYLK
VLQRKQMARAKEVNEKEEGNNNYIQFIRDVVVWAFGAYLENKLKNYKNEL
QPPLSKENIGLNDTLKELFPEEKVKSPFNIKCRFSISTFIDNKGKSTDNT
SAEAVKTDGKEDEKDKKNIKRKDLLCFYLFLRLLDENEICKLQHQFIKYR
CSLKERRFPGNRTKLEKETELLAELEELMELVRFTMPSIPEISAKAESGY
DTMIKKYFKDFIEKKVFKNPKTSNLYYHSDSKTPVTRKYMALLMRSAPLH
LYKDIFKGYYLITKKECLEYIKLSNIIKDYQNSLNELHEQLERIKLKSEK
QNGKDSLYLDKKDFYKVKEYVENLEQVARYKHLQHKINFESLYRIFRIHV
DIAARMVGYTQDWERDMHFLFKALVYNGVLEERRFEAIFNNNDDNNDGRI
VKKIQNNLNNKNRELVSMLCWNKKLNKNEFGAIIWKRNPIAHLNHFTQTE
QNSKSSLESLINSLRILLAYDRKRQNAVTKTINDLLLNDYHIRIKWEGRV
DEGQIYFNIKEKEDIENEPIIHLKHLHKKDCYIYKNSYMFDKQKEWICNG
IKEEVYDKSILKCIGNLFKFDYEDKNKSSANPKHT
A non-limiting example of a Cas13d protein
is Ruminoccocus Flavefaciens (CasRx), 966 aa:
(SEQ ID NO: 17)
MIEKKKSFAKGMGVKSTLVSGSKVYMTTFAEGSDARLEKIVEGDSIRSVN
EGEAFSAEMADKNAGYKIGNAKFSHPKGYAVVANNPLYTGPVQQDMLGLK
ETLEKRYFGESADGNDNICIQVIHNILDIEKILAEYITNAAYAVNNISGL
DKDIIGFGKFSTVYTYDEFKDPEHHRAAFNNNDKLINAIKAQYDEFDNFL
DNPRLGYFGQAFFSKEGRNYIINYGNECYDILALLSGLRHWVVHNNEEES
RISRTWLYNLDKNLDNEYISTLNYLYDRITNELTNSFSKNSAANVNYIAE
TLGINPAEFAEQYFRFSIMKEQKNLGFNITKLREVMLDRKDMSEIRKNHK
VFDSIRTKVYTMMDFVIYRYYIEEDAKVAAANKSLPDNEKSLSEKDIFVI
NLRGSFNDDQKDALYYDEANRIWRKLENIMHNIKEFRGNKTREYKKKDAP
RLPRILPAGRDVSAFSKLMYALTMFLDGKEINDLLTTLINKFDNIQSFLK
VMPLIGVNAKFVEEYAFFKDSAKIADELRLIKSFARMGEPIADARRAMYI
DAIRILGTNLSYDELKALADTFSLDENGNKLKKGKHGMRNFIINNVISNK
RFHYLIRYGDPAHLHEIAKNEAVVKFVLGRIADIQKKQGQNGKNQIDRYY
ETCIGKDKGKSVSEKVDALTKIITGMNYDQFDKKRSVIEDTGRENAEREK
FKKIISLYLTVIYHILKNIVNINARYVIGFHCVERDAQLYKEKGYDINLK
KLEEKGFSSVTKLCAGIDETAPDKRKDVEKEMAERAKESIDSLESANPKL
YANYIKYSDEKKAEEFTRQINREKAKTALNAYLRNTKWNVIIREDLLRID
NKTCTLFRNKAVHLEVARYVHAYINDIAEVNSYFQLYHYIMQRIIMNERY
EKSSGKVSEYFDAVNDEKKYNDRLLKLLCVPFGYCIPRFKNLSIEALFDR
NEAAKFDKEKKKVSGN
Ruminoccocus Flavefaciens Cas13d protein
(CasRx), 2901 nt, is encoded by:
(SEQ ID NO: 18)
ATGATCGAAAAAAAAAAGTCCTTCGCCAAGGGCATGGGCGTGAAGTCCAC
ACTCGTGTCCGGCTCCAAAGTGTACATGACAACCTTCGCCGAAGGCAGCG
ACGCCAGGCTGGAAAAGATCGTGGAGGGCGACAGCATCAGGAGCGTGAAT
GAGGGCGAGGCCTTCAGCGCTGAAATGGCCGATAAAAACGCCGGCTATAA
GATCGGCAACGCCAAATTCAGCCATCCTAAGGGCTACGCCGTGGTGGCTA
ACAACCCTCTGTATACAGGACCCGTCCAGCAGGATATGCTCGGCCTGAAG
GAAACTCTGGAAAAGAGGTACTTCGGCGAGAGCGCTGATGGCAATGACAA
TATTTGTATCCAGGTGATCCATAACATCCTGGACATTGAAAAAATCCTCG
CCGAATACATTACCAACGCCGCCTACGCCGTCAACAATATCTCCGGCCTG
GATAAGGACATTATTGGATTCGGCAAGTTCTCCACAGTGTATACCTACGA
CGAATTCAAAGACCCCGAGCACCATAGGGCCGCTTTCAACAATAACGATA
AGCTCATCAACGCCATCAAGGCCCAGTATGACGAGTTCGACAACTTCCTC
GATAACCCCAGACTCGGCTATTTCGGCCAGGCCTTTTTCAGCAAGGAGGG
CAGAAATTACATCATCAATTACGGCAACGAATGCTATGACATTCTGGCCC
TCCTGAGCGGACTGAGGCACTGGGTGGTCCATAACAACGAAGAAGAGTCC
AGGATCTCCAGGACCTGGCTCTACAACCTCGATAAGAACCTCGACAACGA
ATACATCTCCACCCTCAACTACCTCTACGACAGGATCACCAATGAGCTGA
CCAACTCCTTCTCCAAGAACTCCGCCGCCAACGTGAACTATATTGCCGAA
ACTCTGGGAATCAACCCTGCCGAATTCGCCGAACAATATTTCAGATTCAG
CATTATGAAAGAGCAGAAAAACCTCGGATTCAATATCACCAAGCTCAGGG
AAGTGATGCTGGACAGGAAGGATATGTCCGAGATCAGGAAAAATCATAAG
GTGTTCGACTCCATCAGGACCAAGGTCTACACCATGATGGACTTTGTGAT
TTATAGGTATTACATCGAAGAGGATGCCAAGGTGGCTGCCGCCAATAAGT
CCCTCCCCGATAATGAGAAGTCCCTGAGCGAGAAGGATATCTTTGTGATT
AACCTGAGGGGCTCCTTCAACGACGACCAGAAGGATGCCCTCTACTACGA
TGAAGCTAATAGAATTTGGAGAAAGCTCGAAAATATCATGCACAACATCA
AGGAATTTAGGGGAAACAAGACAAGAGAGTATAAGAAGAAGGACGCCCCT
AGACTGCCCAGAATCCTGCCCGCTGGCCGTGATGTTTCCGCCTTCAGCAA
ACTCATGTATGCCCTGACCATGTTCCTGGATGGCAAGGAGATCAACGACC
TCCTGACCACCCTGATTAATAAATTCGATAACATCCAGAGCTTCCTGAAG
GTGATGCCTCTCATCGGAGTCAACGCTAAGTTCGTGGAGGAATACGCCTT
TTTCAAAGACTCCGCCAAGATCGCCGATGAGCTGAGGCTGATCAAGTCCT
TCGCTAGAATGGGAGAACCTATTGCCGATGCCAGGAGGGCCATGTATATC
GACGCCATCCGTATTTTAGGAACCAACCTGTCCTATGATGAGCTCAAGGC
CCTCGCCGACACCTTTTCCCTGGACGAGAACGGAAACAAGCTCAAGAAAG
GCAAGCACGGCATGAGAAATTTCATTATTAATAACGTGATCAGCAATAAA
AGGTTCCACTACCTGATCAGATACGGTGATCCTGCCCACCTCCATGAGAT
CGCCAAAAACGAGGCCGTGGTGAAGTTCGTGCTCGGCAGGATCGCTGACA
TCCAGAAAAAACAGGGCCAGAACGGCAAGAACCAGATCGACAGGTACTAC
GAAACTTGTATCGGAAAGGATAAGGGCAAGAGCGTGAGCGAAAAGGTGGA
CGCTCTCACAAAGATCATCACCGGAATGAACTACGACCAATTCGACAAGA
AAAGGAGCGTCATTGAGGACACCGGCAGGGAAAACGCCGAGAGGGAGAAG
TTTAAAAAGATCATCAGCCTGTACCTCACCGTGATCTACCACATCCTCAA
GAATATTGTCAATATCAACGCCAGGTACGTCATCGGATTCCATTGCGTCG
AGCGTGATGCTCAACTGTACAAGGAGAAAGGCTACGACATCAATCTCAAG
AAACTGGAAGAGAAGGGATTCAGCTCCGTCACCAAGCTCTGCGCTGGCAT
TGATGAAACTGCCCCCGATAAGAGAAAGGACGTGGAAAAGGAGATGGCTG
AAAGAGCCAAGGAGAGCATTGACAGCCTCGAGAGCGCCAACCCCAAGCTG
TATGCCAATTACATCAAATACAGCGACGAGAAGAAAGCCGAGGAGTTCAC
CAGGCAGATTAACAGGGAGAAGGCCAAAACCGCCCTGAACGCCTACCTGA
GGAACACCAAGTGGAATGTGATCATCAGGGAGGACCTCCTGAGAATTGAC
AACAAGACATGTACCCTGTTCAGAAACAAGGCCGTCCACCTGGAAGTGGC
CAGGTATGTCCACGCCTATATCAACGACATTGCCGAGGTCAATTCCTACT
TCCAACTGTACCATTACATCATGCAGAGAATTATCATGAATGAGAGGTAC
GAGAAAAGCAGCGGAAAGGTGTCCGAGTACTTCGACGCTGTGAATGACGA
GAAGAAGTACAACGATAGGCTCCTGAAACTGCTGTGTGTGCCTTTCGGCT
ACTGTATCCCCAGGTTTAAGAACCTGAGCATCGAGGCCCTGTTCGATAGG
AACGAGGCCGCCAAGTTCGACAAGGAGAAAAAGAAGGTGTCCGGCAATTA
A

Cas12 and Cas13 proteins, and their variants, may be produced by recombinant expression using well-known methodologies of molecular biology, or obtained commercially.

Methods and compositions of the present invention are not limited to particular amino acid sequences identified herein and variants of a reference peptide or protein are encompassed.

Variants of a peptide or protein described herein are characterized by conserved functional properties compared to the corresponding peptide or protein.

As disclosed herein, many Cas12 and Cas13 proteins and variants thereof are known and can be used in assays according to aspects of the present disclosure. Variant Cas12 and Cas13 proteins have at least 30%, 35%, 40%, 45%, 50%, 55%, 60%, 65%, 70%, 75%, 80%, 85%, 90% 95%, 96%, 97%, 98%, 99%, or greater identity to a particular reference Cas12 or Cas13 protein and retains the desired functional abilities of a reference Cas12 or Cas13 protein, including capability to form a complex with a crRNA and corresponding target nucleic acid, and to cleave “off-target” nucleic acids including a reporter nucleic acid.

Non-limiting, example amino acid sequences of Cas12 and Cas13 proteins are included herein. Variant of these or other Cas12 and Cas13 proteins have at least 30%, 35%, 40%, 45%, 50%, 55%, 60%, 65%, 70%, 75%, 80%, 85%, 90% 95%, 96%, 97%, 98%, 99%, or greater identity to a particular reference Cas12 or Cas13 protein and retains the desired functional abilities of a reference Cas12 or Cas13 protein, including capability to form a complex with a crRNA and corresponding target nucleic acid, and to cleave “off-target” nucleic acids including a reporter nucleic acid.

Percent identity is determined by comparison of amino acid or nucleic acid sequences, including a reference amino acid or nucleic acid sequence and a putative homologue amino acid or nucleic acid sequence. To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions ×100%). The two sequences compared are generally the same length or nearly the same length. A variant may be a naturally-occurring variant of a reference protein such as an ortholog expressed by another organism or species of organism.

The determination of percent identity between two sequences can also be accomplished using a mathematical algorithm. Algorithms used for determination of percent identity illustratively include the algorithms of S. Karlin and S. Altshul, PNAS, 90:5873-5877, 1993; T. Smith and M. Waterman, Adv. Appl. Math. 2:482-489, 1981, S. Needleman and C. Wunsch, J. Mol. Biol., 48:443-453, 1970, W. Pearson and D. Lipman, PNAS, 85:2444-2448, 1988 and others incorporated into computerized implementations such as, but not limited to, GAP, BESTFIT, FASTA, TFASTA; and BLAST, for example incorporated in the Wisconsin Genetics Software Package, Genetics Computer Group, 575 Science Drive, Madison, Wis.) and publicly available from the National Center for Biotechnology Information.

A non-limiting example of a mathematical algorithm utilized for the comparison of two sequences is the algorithm of Karlin and Altschul, 1990, PNAS 87:2264-2268, modified as in Karlin and Altschul, 1993, PNAS. 90:5873-5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, word length=12 to obtain nucleotide sequences homologous to a nucleic acid molecules of the present invention. BLAST protein searches are performed with the XBLAST program parameters set, e.g., to score 50, word length=3 to obtain amino acid sequences homologous to a protein molecule of the present invention. To obtain gapped alignments for comparison purposes, Gapped BLAST are utilized as described in Altschul et al., 1997, Nucleic Acids Res. 25:3389-3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used. Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

One of skill in the art will recognize that one or more amino acid mutations can be introduced without altering the functional properties of a given nucleic acid or protein, respectively. Mutations can be introduced using standard molecular biology techniques, such as site-directed mutagenesis and PCR-mediated mutagenesis, to produce variants. For example, one or more amino acid substitutions, additions, or deletions can be made without altering the functional properties of a reference protein.

Conservative amino acid substitutions can be made or may be present in reference proteins to produce or identify variants.

Conservative amino acid substitutions are art recognized substitutions of one amino acid for another amino acid having similar characteristics. For example, each amino acid may be described as having one or more of the following characteristics: electropositive, electronegative, aliphatic, aromatic, polar/nonpolar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size; alanine, cysteine, aspartate, glycine, asparagine, proline, threonine, serine, valine are all typically considered to be small.

A variant can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

A non-activated RNP is formed by contacting a crRNA and Cas 12 or Cas 13 protein. The non-activated RNP is then contacted with the target nucleic acid sequence under conditions allowing specific binding of the non-activated RNP to the target nucleic acid sequence, forming an activated RNP. The activated RNP indiscriminately cleaves the reporter nucleic acid.

According to aspects of the present disclosure, an assay for a target nucleic acid analyte can be quantitative or qualitative. A quantitative assay provides a count of the number of target nucleic acid molecules in a sample, whereas a qualitative assay provides a positive/negative result indicating presence/absence of a target nucleic acid at a specified % confidence level within a specified time.

As described herein, the time needed to determine the presence/absence of a target nucleic acid with a particular confidence level, such as 95% confidence level, or lower, or higher confidence level can be calculated. For any fixed nanopore reading time Tₘ, one can solve for the maximal λₚ and therefore the minimal required reaction time Tᵣ. FIG. 11B illustrates the total experimental time (Tᵣ+Tₘ) as a function of target nucleic acid and activated RNP concentrations. Using a selected fixed RNP concentration, the total experimental time can be significantly reduced when target nucleic acid concentration is increased up to that of activated RNP concentration, beyond which the total experimental time is independent of the target nucleic acid concentration. This is because the rate constant k is determined by the smaller amount between target nucleic acid and RNP. Second, although when the activated RNP concentration is higher than target nucleic acid, the constant rate k is independent of activated RNP concentration, the total experimental time can be significantly increased when activated RNP target concentration is increased. This stems from the fact that the electrophoretic mobility of the reporters decays exponentially as the activated RNP concentration is increased. Hence, more time is needed in the nanopore reading to make a call at 95% confidence. An optimized combination of target nucleic acid concentration and activated RNP concentration provides for a positive/negative call at 95% confidence which can be made within an hour. It was found that the activated RNP concentration between 10-100 nM and target nucleic acid concentration higher than 10 nM provides for a positive/negative call at 95% confidence which can be made within an hour. Optionally, an amplification step, such as PCR can be used to increase the amount of the target nucleic acid sequence. For example, if the starting target nucleic acid sequence concentration is less than 10 nM, an amplification step can be used before adding the sample to the first chamber to increase the concentration of the target nucleic acid sequence.

Alternatively, the assay time can be increased or decreased and concentrations of activated RNP and target nucleic acid adjusted accordingly.

According to aspects of the present disclosure, the non-activated RNP concentration is in a range of about 1 nM-10 uM, such as 10-100 nM. The components of the non-activated RNP are present in a ratio of at least 1:1 such that each component is also present in a concentration of about 1 nM-10 uM, such as 10-100 nM.

Nucleic acid reporter concentration is in the range of about 10 picomolar (pM) to about 1 micromolar (μM), such as about 50 pM to about 250 nanomolar (nM).

According to aspects of the present disclosure, an assay for detection of a nucleic acid analyte includes: 1) formation of a non-activated RNP complex; 2) formation of an activated RNP complex; and 3) indiscriminate cleavage of a nucleic acid reporter in the presence of the activated RNP complex.

Formation of the non-activated RNP complex is achieved by contacting a Cas12 or Cas13 protein with a crRNA under appropriate conditions. Appropriate conditions for formation of a non-activated RNP complex are typically “physiological” conditions, such as in an aqueous medium containing physiological salt concentrations, pH, and at a temperature in the range of about 4° C. to about 37° C., preferably “room temperature” (about 25° C.) to about 37° C., depending on the desired time for formation of the non-activated RNP complex. Phosphate-buffered saline (1×PBS) is a non-limiting example of a suitable aqueous medium containing physiological salt concentrations and physiological pH. At room temperature, non-activated RNP complex is formed in about 5 minutes to about 60 minutes, such as about 15 minutes to about 30 minutes.

Formation of activated RNP complex is achieved by mixing the non-activated RNP with target nucleic acid, double-stranded target DNA for Cas12 and single-stranded target RNA for Cas13 under appropriate conditions.

Appropriate conditions for formation of a non-activated RNP complex are typically “physiological” conditions, such as in an aqueous medium containing physiological salt concentrations, pH, and at a temperature in the range of about 4° C. to about 37° C., preferably “room temperature” (about 25° C.) to about 37° C., depending on the desired time for formation of the non-activated RNP complex. Phosphate-buffered saline (1×PBS) is a non-limiting example of a suitable aqueous medium containing physiological salt concentrations and physiological pH. At 37° C., non-activated RNP complex is formed in about 5 minutes to about 60 minutes, such as about 10 minutes to about 20 minutes.

The nucleic acid reporter is then incubated with the activated RNP complex under appropriate conditions to produce indiscriminate cleavage of nucleic acids by the “off-target” activity of the activated RNP complex, which includes cleavage of the nucleic acid reporter. Appropriate conditions for cleavage include an aqueous Cas enzyme activity-compatible medium containing a divalent cation, preferably Mg⁺⁺, in an amount sufficient for Cas activity, such as about 1-10 mM MgCl₂, such as about 2.5-75 mM MgCl₂, such as about 5 mM MgCl₂; a salt, such as NaCl or KCl, in a concentration in the range of at least 1 mm and less than 200 mM, a buffer compatible with cleavage activity such as, but not limited to HEPES, substantially protein free, a pH in the range appropriate for the particular buffer, generally in the range of about pH 5 to about pH 9, such as about pH 6 to about pH 8, and at a temperature in the range of about 4° C. to about 37° C., preferably “room temperature” to about 37° C., depending on the desired time for cleavage of the reporter if the target nucleic acid is present. A non-limiting example of a aqueous Cas enzyme activity-compatible medium includes about 40-200 mM NaCl, such as 50-150 mM NaCl, such as 75-100 mM NaCl; about 1-10 mM MgCl₂, such as about 2.5-75 mM MgCl₂, such as about 5 mM MgCl₂; about 10-30 mM HEPES buffer, such as about 20 mM HEPES buffer, about 0.05-5 mM EDTA, such as about 0.1-0.2 mM EDTA, at a pH of about 6.5 at 25° C. At 37° C., the cleavage reaction time can be in the range of about 1 minute to about 60 minutes, such as about 5 minutes to about 30 minutes.

The reaction is then terminated, for example by adding sufficient salt to terminate the reaction and such that the final salt concentration is about 0.5 M to 50 M, for efficient nanopore counting of the remaining nucleic acid reporter. Thus, the reaction is terminated by adding a high concentration of salt, such as KCl or NaCl, such that the final salt concentration is in the range 0.5 M to 50 M, thereby stopping the reaction and providing an ionic environment for the nanopore detection.

Results show that the reporter cleavage rate constant is proportional to the target nucleic acid concentration.

Cleavage of the reporter nucleic acid reduces passage of the reporter nucleic acid through the nanopore such that a reduction of resistive pulses is produced which provides a signal representative of presence of the target nucleic acid in the sample.

Embodiments of inventive compositions and methods are illustrated in the following examples. These examples are provided for illustrative purposes and are not considered limitations on the scope of inventive compositions and methods.

### EXAMPLES

Sequence-Specific Recognition of Virus DNA with Solid-State CRISPR-Cas12a-Assisted Nanopores (SCAN)

Materials and Methods

A.s.Cas12a Ultra (#10001272) and IDTE pH7.5 buffer (#11-01-02-02) were purchased from Integrated DNA Technologies (IDT). dsDNA target nucleic acid and crRNA were also synthesized and purchased from IDT. M13mp18 ssDNA (#N4040S) and NEBuffer 3.1 (#B7203S) were purchased from NEW ENGLAND Biolabs Inc. (NEB). DNA elution buffer was purchased from Zymo Research (#D4036-5). Nuclease-free molecular Biology grade water was purchased from Hyclone (SH30538). DPBS was purchased from Thermo Fisher (#14190250). DNA gel loading dye (6×) was purchased from Thermo Fisher (#R0611). 10× IDT reaction buffer (200 mM HEPES, 1 M NaCl, 50 mM MgCl₂ and 1 mM EDTA, PH6.5@25° C.) was made at the lab. MgCl₂, NaCl, KCl and Tris-EDTA-buffer solution (10 mM Tris-HCl and 1 mM EDTA) were purchased from Sigma-Aldrich. HEPES was purchased from Gibco #15630-080. Agarose was purchased from Bio-rad (#1613102). Ethidium Bromide (EB) was purchased from Life Technologies (#15585011). DNA Ladder was purchased from Thermo Scientific (#SM0311). Ag/AgCl wires electrodes were fabricated by using 0.2 mm Ag wires (Warner Instruments, Hamden, USA). Micro injectors of 34 gauge were purchased from World Precision Instruments. Piranha solution was made by mixing concentrated sulfuric acid (H₂SO₄) with hydrogen peroxide (H₂O₂). Quartz capillaries with inner and outer diameter of 0.5 mm and 1 mm were purchased from Sutter Instrument.

Virus Detection Assay

Aligned sequences of three domains of HIV-1, which are GAG (capsid protein), POL (protease, reverse transcriptase and integrase), and ENV (glycoprotein) were obtained from an HIV sequence database detailed in Rouzioux, C. et al., Retrovirology 2018, 15(1), 30. Shannon entropy value of each nucleotide of the aligned sequences was calculated with the “Entropy-One” function in the HIV sequence database. Two HIV-1 DNA oligonucleotides from the GAG region (HIV-1 Target 1 and Target 2) and two specific crRNAs were synthesized by IDT. Sequences used in this example are as follows:

Target 1: 5′-tatcacctagaacTTTAAATGCATGGGTAAAAGTAGTAgaagagaaggct-3′ (SEQ ID NO:1). The underlined portion is the PAM, upper case is the target nucleic acid sequence, lower case sequence is included to show context in the virus genome.

crRNA1: 5′-UAAUUUCUACUCUUGUAGAUAAUGCAUGGGUAAAAGUAGUA-3′ (SEQ ID NO:2) In this sequence, the first 20 nt, UAAUUUtCUACUCUUGUAGAU (SEQ ID NO:5), constitute the direct repeat, and the remainder, AAUGCAUGGGUAAAAGUAGUA (SEQ ID NO:6), is the target-specific nucleic acid sequence.

Target 2: 5′-ccTTTAACTTCCCTCAGGTCACTCTTTGGcaacgacccctcgtcacaataa-3′ (SEQ ID NO:3). The underlined portion is the PAM, upper case is the target nucleic acid sequence, lower case sequence is included to show context in the virus genome.

crRNA2: 5′-UAAUUUCUACUCUUGUAGAUACUUCCCUCAGGUCACUCUUUGG-3′ (SEQ ID NO:4) In this sequence, the first 20 nt, UAAUUUCUACUCUUGUAGAU (SEQ ID NO:5), constitute the direct repeat, and the remainder, ACUUCCCUCAGGUCACUCUUUGC (SEQ ID NO:7), is the target-specific nucleic acid sequence.

The synthesized HIV-1 DNA oligonucleotides were resuspended in molecular biology grade water, annealed in DNA elution buffer. The crRNAs were resuspended in IDTE pH 7.5 buffer and stored in −80° C. For ribonucleoprotein (RNP) complex formation, Cas12a and crRNA were mixed in 1×PBS to form the non-activated RNP complex at room temperature for 20 min and stored in −80° C. In the cleavage reaction, non-activated RNP complex was mixed with dsDNA target and incubated at 37° C. for 10 min for RNP activation, producing activated RNP complex. Then ssDNA reporters were added and incubated at 37° C. for cleavage. After the reaction, results were examined both in agarose gel and in the nanopore device. For gel imaging, reactions were terminated with DNA loading dye (6×). The 24 μl mixture was loaded to EB-stained 1% (wt/vol) agarose gel for electrophoresis analysis. For nanopore analysis, reactions were terminated by adjusting the salt concentrations to 1M KCl.

Glass Nanopore Fabrication

To remove organic residues from quartz capillaries, as-purchased quartz capillaries were firstly cleaned in Piranha solution for 30 minutes, then rinsed with DI water, and dried in a vacuum oven at 120° C. for 15 min. A two-line recipe, (1) Heat 750, Filament 5, Velocity 50, Delay140, and Pull 50; (2) Heat 710, Filament 4, Velocity 30, Delay 155, and Pull 215, was used to pull the capillaries with a laser pipette puller (P-2000, Sutter Instruments, USA). This recipe typically produces nanopores of diameter around 10 nm.

Nanopore Sensing and Data Analysis

A constant voltage was applied across the glass nanopore by 6363 DAQ card (National Instruments, USA). A transimpedance amplifier (Axopatch 200B, Molecular Device, USA) was used to amplify the resulting current and then digitalized by the 6363 DAQ card at 100 kHz sampling rate. Finally, a customized MATLAB (MathWorks) software was used to analyze the current time trace and extract the single molecule translocation information.

Results

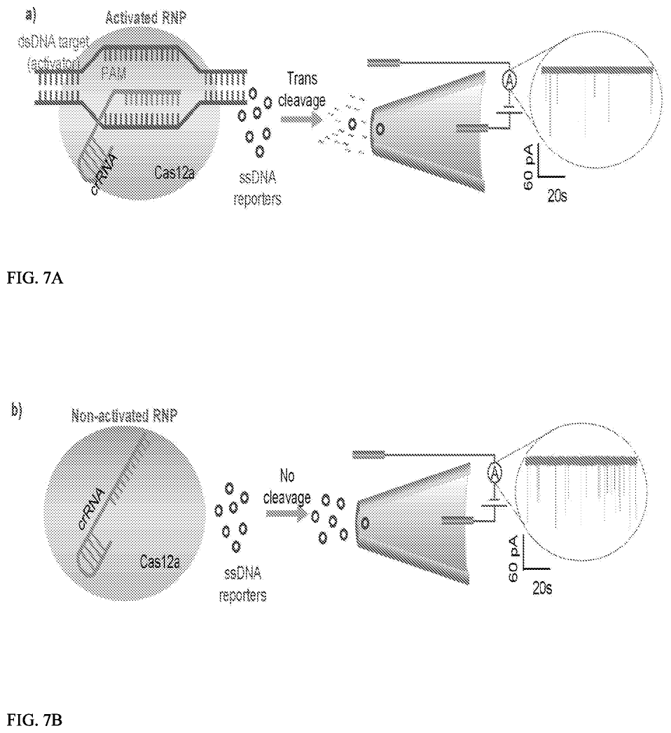

FIG. 7A and FIG. 7B schematically show aspects of a Solid-State CRISPR-Cas12a- or CRISPR-Cas13-Assisted Nanopore (SCAN) sensor method of the present disclosure, used in this example.

In this example of a SCAN method, circular ssDNAs (M13mp18, 7249 bases) of a known concentration (typically 100 pM) were used as reporters. If target HIV-1 DNAs exist in the analyte solution, the Cas12a/crRNA complex (i.e., non-activated RNP) is activated by binding specifically to the target HIV-1 DNAs, producing an activated RNP complex, see FIG. 7A. The activated RNP complex is then able to digest the ssDNA reporters. As a result, the effective concentration of the circular ssDNA reporter is reduced. On the other hand, if the target HIV-1 DNAs are not present in the analyte solution, the RNP complex remains inactive and will not degrade the ssDNA reporter, see FIG. 7B. Thus, the abundance of the remaining circular ssDNA reporter indicates the existence/absence of the target DNAs such that presence of the target HIV-1 DNAs in the analyte solution detectably reduces the ssDNA reporter event rate through the nanopore sensors.

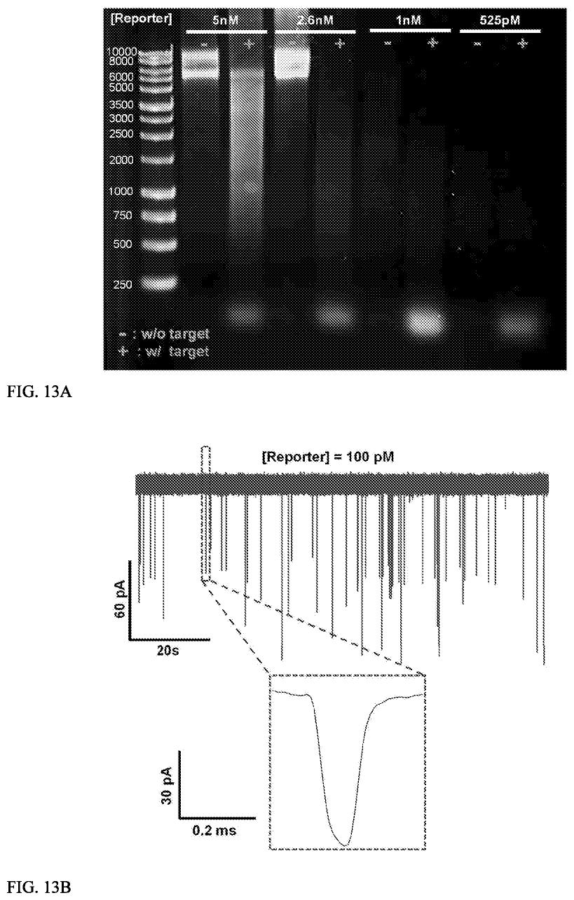

It is noted that, while the remaining ssDNA reporter can be readily visualized by conventional gel electrophoresis in this example, the nanopore readout is much more sensitive and can be performed in-situ, see FIGS. 13A and 13B.

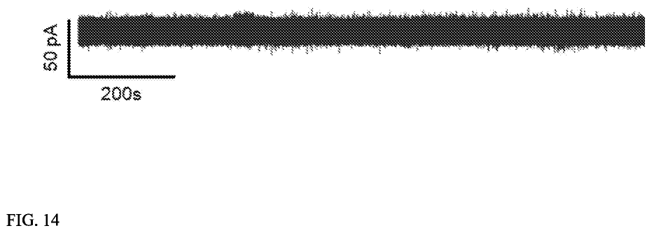

To ensure all events observed in the nanopore sensors correspond to the ssDNA reporter rather than interfering background molecules (e.g., RNPs), a control nanopore experiment was performed using a pure RNP and HIV-1 DNA sample, 30 nM each, without any ssDNA reporter. Not even a single event was observed for a measurement time of 1000 seconds, see FIG. 14, which confirms that the translocation rate of the background activated RNPs is less than 0.001 s⁻¹.

HIV-1 Assay and Buffer Optimization

For sequence-specific recognition of HIV-1 DNA, the crRNAs targeted conserved regions in all HIV-1 subtypes. This example focused on three commonly evaluated domains of HIV-1, which are GAG (capsid protein), POL (protease, reverse transcriptase and integrase), and ENV (glycoprotein), detailed for example in Zhao, J. et al., European Journal of Clinical Microbiology & Infectious Diseases 2019, 38 (5), 829-842; Schlatzer, D. et al., Analytical chemistry 2017, 89 (10), 5325-5332; and Waheed, A. A. et al., AIDS research and human retroviruses 2012, 28 (1), 54-75. Two 50 bp dsDNAs from the GAG region were synthesized as HIV-1 targets. Two specific crRNAs were designed for each of these dsDNAs targets as shown herein.

Three candidate buffers were tested: NEBuffer 3. 1, PBS buffer and IDT buffer, see Table 2 for detailed compositions.

TABLE 2
Detailed compositions of NEBuffer 3.1, PBS buffer and IDT buffer

| Buffer Name | Composition |
|---|---|
| NEBuffer 3.1 (1X) | 100 mM NaCl |
| | 50 mM Tris-HCl |
| | 10 mM MgCl₂ |
| | 100 μg/ml Bovine Serum Albumin (BSA) |
| | pH 7.9@25° C. |
| PBS buffer (1X) | 137 mM NaCl |
| | 2.7 mM KCl |
| | 10 mM Na₂HPO₄ |
| | 1.8 mM KH₂PO₄ |
| | pH 7.4@25° C. |
| IDT buffer (1X) | 100 mM NaCl |
| | 5 mM MgCl₂ |
| | 20 mM HEPES |
| | 0.1 mM EDTA |
| | pH 6.5@25° C. |

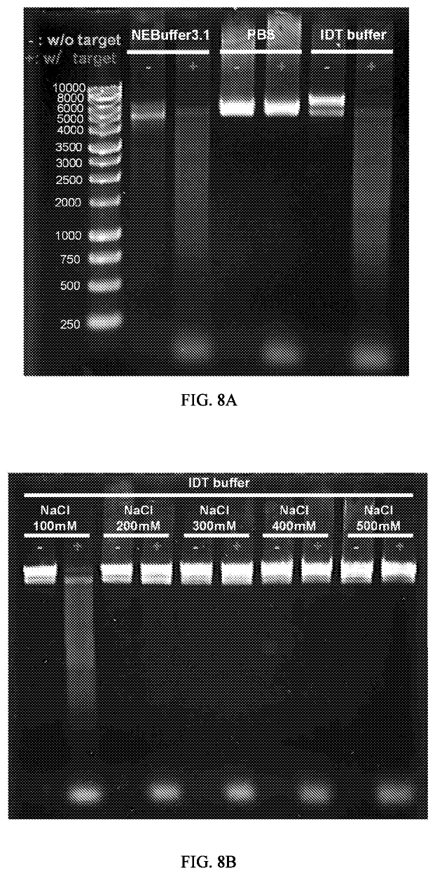

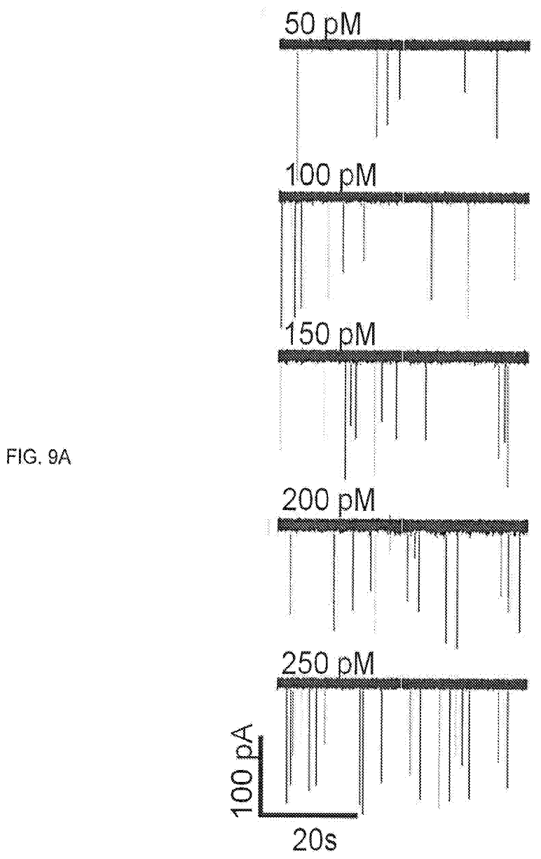

A gel analysis was performed to validate each of these buffers, and results are shown in FIG. 8A. As shown, the commonly used NEBuffer 3.1 indeed worked in the nucleic acid assay. However, it is incompatible with the nanopore sensor. The presence of high concentration (1.5 μM) of Bovine Serum Albumin (BSA) in the NEBuffer acts as an opposing obstacle for ssDNA reporters and dramatically impacted the nanopore ssDNA reporter event rates. For the PBS buffer, the gel results showed that it did not support the cleavage activity of Cas12a. This is likely due to the lack of Mg²⁺ ions, the key co-factor in Cas12a enzymatic activities.

It was hypothesized that a buffer with Mg²⁺ ions that has no BSA is desirable for the SCAN device. The IDT buffer is such a candidate. Functionality of IDT buffer, containing Mg2+ ions and no BSA, in a nucleic acid assay according to aspects of the present disclosure was determined in this example, and results are shown in FIG. 8A. While Cas12a indeed functioned properly in the IDT buffer, the low salt concentration of the buffer, 100 mM, is non-ideal for the nanopore sensing. Therefore, salt concentration in the IDT buffer was evaluated to determine the impact on the Cas12a assay of this example. It was found that 200 mM salt would start killing the activity of the Cas12a, see FIG. 8B. Thus, the IDT buffer with 100 mM salt can be used in the nucleic acid Cas12a reaction. To solve the conflicting buffer requirements in the Cas12a reaction (low salt) and the nanopore sensing (high salt), a two-step protocol was used in a SCAN system and method according to aspects of the present disclosure. The reaction between the target DNA, Cas12a/crRNA, and ssDNA reporter was performed in the IDT buffer. The reaction was then terminated by adding 1.045 M KCl solution such that the final salt concentration is 1 M for efficient nanopore counting of the remaining ssDNA reporter.

Nanopore Event Rate for Circular ssDNA Reporter Quantification

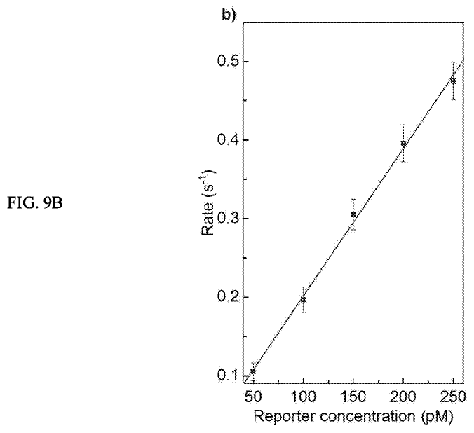

For a typical SCAN experiment, the RNP concentration remains constant during the Cas12a cleavage reaction. To validate if the nanopore event rate can be used as a quantitative readout for the ssDNA reporter concentration at the constant RNP background, nanopore counting experiments with serially diluted ssDNA reporter were performed. In all experiments, the RNP and salt concentration was fixed as 30 nM and 1 M, respectively. FIG. 9A shows the time traces of the ionic current from each of these cases. The results show that events occur more often as the ssDNA reporter concentration increases. The extracted event rate as a function of the reporter concentration was plotted in FIG. 9B. A clear linear relationship between the event rate and the ssDNA reporter concentration was observed (R²=0.98), which validates the abundance of ssDNA reporter can be quantified by nanopore counting at the constant RNP background.

Virus Nucleic Acid-Activated Cas12a Trans-Cleavage Monitored by Nanopore Counting

Having shown the linear relationship between the ssDNA reporter and the nanopore event rate under the constant RNP, an assay for viral target nucleic acid was performed using a SCAN method and apparatus according to aspects of the present disclosure.

Three different HIV-1 target nucleic acid concentrations, 15, 30, and 60 nM, were tested by adding dsDNA sample to the RNP solutions in the IDT buffer. In all the experiments, the initial ssDNA reporter concentration was fixed at 100 pM. The reaction was terminated at various reaction times, 0, 5, 10, 20, and 30 minutes, by adding KCl salt to the final salt concentration of 1M. The remaining ssDNA reporter concentration was measured by the calibration-free nanopore counting method as described below under the heading “Linking ssDNA reporter concentration with translocation rate.”

Linking ssDNA Reporter Concentration with Translocation Rate

In the diffusion-limited region, the capture rate for the conical-shaped glass nanopore is given by:

α=2πμ\hat{d}ΔV

where μ is the free solution electrophoretic mobility, ΔV is the applied electric potential across the pore, and \hat{d} is the characteristic length of the nanopore. Also, the baseline current can be estimated as:

I_{b}=2πΛCᵢₒₙ\hat{d}ΔV

where Λ is the molar conductivity which depends on the mobility and valance of the ions as:

Λ=ΣᵢN_{A}ezᵢμᵢ

Thus, the ssDNA reporter translocation rate R=αN_{A}C_{DNA} can be written as:

$$R = \frac{μ {N}_{A} {C}_{D N A}}{Λ {C}_{i o n}} {I}_{b}$$

where N_{A} is the Avogadro constant and C_{DNA} is the ssDNA reporter concentration. Hence, the remaining ssDNA reporter concentration can be estimated based on their translocation rate through the nanopore:

$${C}_{D N A} = \frac{Λ {C}_{i o n}}{μ {N}_{A}} \frac{R a t e}{{I}_{b}}$$

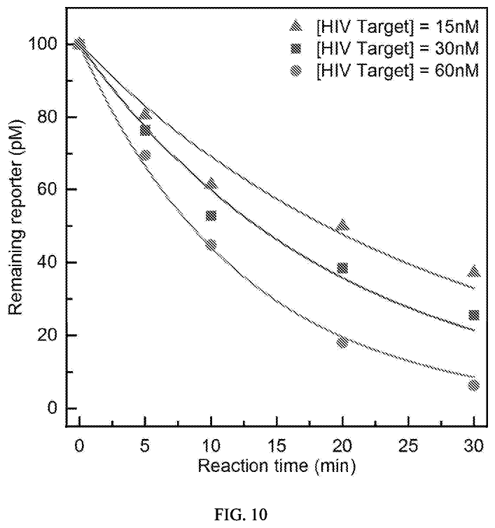

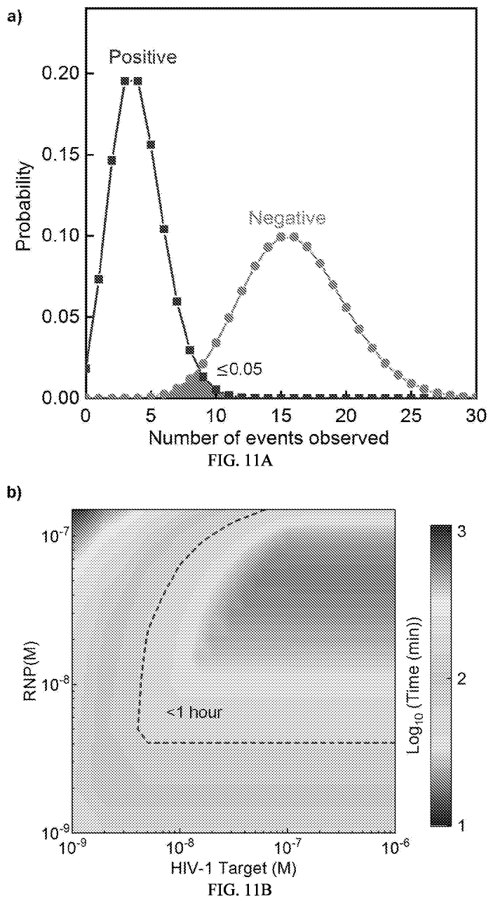

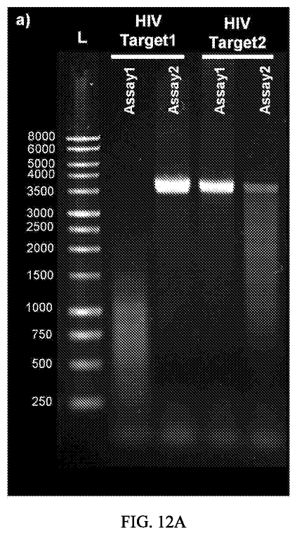

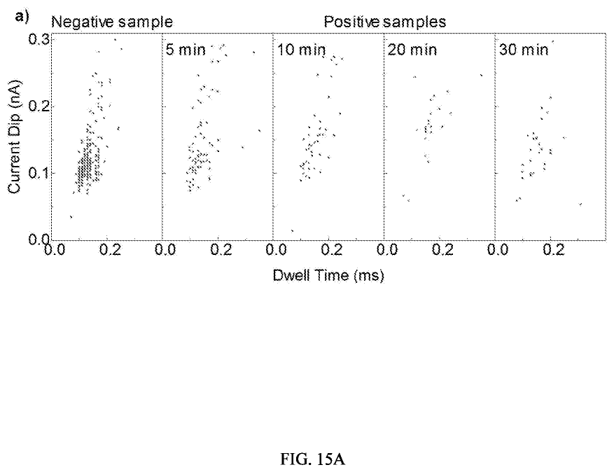

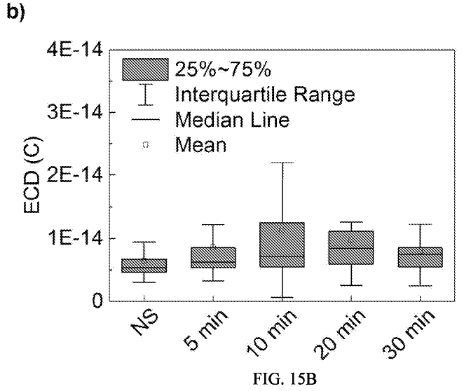

FIG. 10 is a graph showing the remaining ssDNA reporter concentration as a function of the reaction time for different target HIV-1 nucleic acid concentrations. For each target HIV-1 nucleic acid concentration, the ssDNA reporter concentration reduced exponentially and can be well fitted by the Michaelis-Menten kinetics, C=C₀e^{-kT}^{ᵣ}, where C and C₀ are the remaining and initial ssDNA reporter concentration, respectively. Tᵣ is the reaction time and k is the rate constant. The rate constant k went up from 0.037 min⁻¹ at 15 nM target HIV nucleic acid to 0.051 min⁻¹ at 30 nM target HIV nucleic acid, and further to 0.081 min⁻¹ at 60 nM target HIV nucleic acid. It was evident that the more target HIV-1 nucleic acid present in the analyte solution, the faster the ssDNA reporters get degraded by the activated Cas12a. The fact that the extracted remaining reporter decayed exponentially by the reaction time, see FIG. 10, and followed the Michaelis-Menten kinetics, indicates that interference from partially cleaved ssDNA reporter is negligible. The distributions of the current dips, dwell time, and event charge deficits (ECDs) of the events was evaluated for a negative sample and positive samples, see FIG. 15. No significant difference was observed between these two samples, indicating most of the detected events were from the intact circular ssDNA reporter.

Statistical Modeling for Qualitative Positive/Negative Test in SCAN

The translocation of molecules through the nanopore is a Poisson process. Thus inferring the event rate from observing n events in Tₘ will have an uncertainty of (1.96(n)^{1/2})/Tₘ. Thus, longer reaction time Tᵣ and measurement time Tₘ is preferred to make a statistically confident call for a qualitative positive/negative test. However, minimizing the total experimental time (Tᵣ+Tₘ) would be highly desirable towards a fast sample-to-result turnaround.

In order to estimate the total experimental time for a qualitative positive/negative test, a statistical model was developed. For the negative case (i.e., no reporter degradation), the expected number of events in the nanopore in a measurement time of Tₘ is given by

λₙ=αμC₀Tₘ

where μ is the electrophoretic mobility of the ssDNA reporter, α is a constant, and C₀ is the initial ssDNA reporter concentration before the reaction. For the positive case after reaction time Tᵣ, the initial reporter concentration C₀ would decrease to C₀e^{-kT}^{ᵣ} (Michaelis-Menten kinetics) and the expected number of events in the nanopore in a measurement time of Tₘ would be:

λ=αμC₀e^{-kT}^{ᵣ}Tₘ

in which k is the rate constant that is linearly proportional to the activated RNP concentration. The activated RNP concentration is limited by the smaller values between HIV-1 DNA and RNP concentration in the system and can be written as

k=A×min(C_{HIV},C_{RNP})

where A is a constant (0.00148 min⁻¹ nM⁻¹, see Michaelis-Menten kinetics below).

Michaelis-Menten Kinetics

Michaelis-Menten kinetics model describes the relationship between the reaction rate v and the concentration of a substrate C as:

$$v = \frac{d C}{d t} = - \frac{{V}_{m} C}{{K}_{m} + C}$$

where Vₘ and Kₘ represent the maximum rate achieved by the system and substrate concentration at which the reaction rate is half of Vₘ. respectively. For small concentrations, it can be assumed that Kₘ+C=Kₘ:

$$\frac{d C}{d t} = - \frac{{V}_{m}}{{K}_{m}} C$$

By introducing the rate constant

$$k = \frac{{V}_{m}}{{K}_{m}} :$$

C=C₀e^{-kt}

Rate constant k is proportional to activated RNP concentration. For this calculation, the concentration of the activated RNP is assumed to be constant. This is a reasonable assumption because the exemplified assay has three steps. (1) RNP formation, Cas12a, and crRNA were mixed in 1×PBS to form the non-activated RNP at room temperature for 20 min. (2) Non-activated RNP complex was mixed with dsDNA target and incubated at 37° C. for 10 min for RNP activation. (3) Then ssDNA reporters were added and incubated at 37° C. for different times (5, 10, 20, and 30 minutes) for cleavage. In step 2, more dsDNA targets were added to the mix to make sure all the RNP complexes have been activated. As a result, the concentration of the activated Cas12a enzyme was constant when the cleavage process starts.

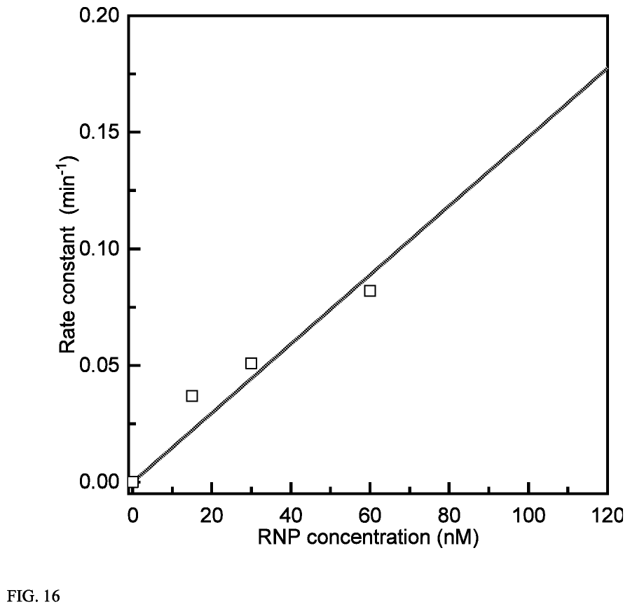

The rate constants at different activated RNP concentrations were obtained by line fitting to the experimental results of 0, 15, 30, and 60 nM, see FIG. 16.

It was found that, while the RNP complexes do not produce measurable events, they do affect the electrophoretic mobility of the ssDNA reporters. It was found the reporter electrophoretic mobility reduces exponentially as the RNP concentration is increased, see “RNP affects the reporter electrophoretic mobility” below.

RNP Affects the Reporter Electrophoretic Mobility

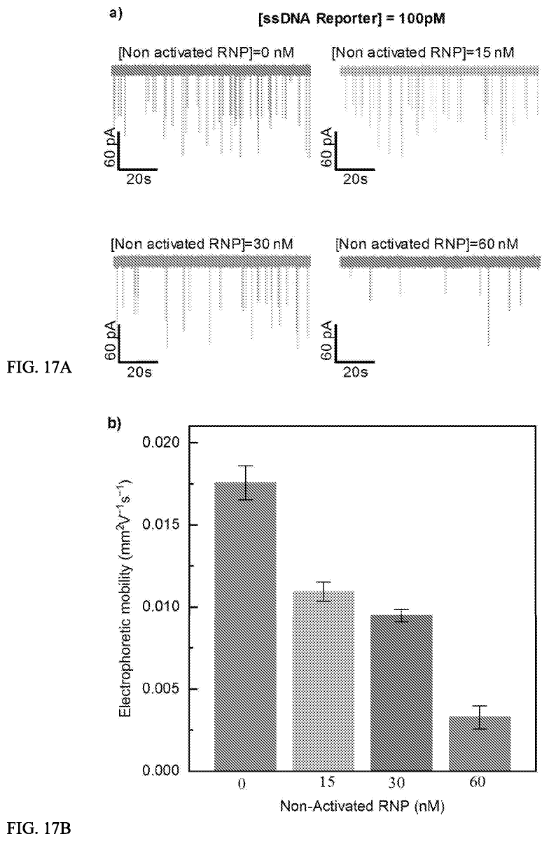

While the RNP complexes do not produce measurable events, they do affect the electrophoretic mobility of the ssDNA reporters. To investigate the effect of RNP concentration on the electrophoretic mobility of the reporter, a nanopore experiment on 100 pM ssDNA reporters was performed at four RNP concentrations, 0, 15, 30, and 60 nM. These RNP were not activated, i.e., no target dsDNA. Based on the time traces of the ionic current in FIG. 17A, translocation events occur less frequently as the RNP concentration increases.

To extract the effective electrophoretic mobility of the ssDNA reporter, a calibration-free nanopore counting method was employed, described herein, to relate the mobility to the molecular event rate and the ionic current baseline. The reduced ssDNA reporter mobility at increased RNP concentration, see FIG. 17B, is likely due to the physical interactions between these two molecules, where RNP complexes act as an opposing obstacle for ssDNA reporters to move.

This phenomenon is described by the Ogston-Morris-Rodbard-Chrambach model, in which molecule electrophoretic mobility has an exponential relationship with the obstacles concentration (μ∝e^{-C}^{_{RNP}}). Hence, electrophoretic mobility of the reporters can be extracted by fitting an exponential curve to the experimental results:

μ=μ₀e^{-βC}^{_{RNP}}

where μ₀ (1.73×10⁻⁸ m² V⁻1 s⁻¹) and β (0.025 nM⁻¹) are the constants of the fitted exponential curve to the experimental results, see Ogston-Morris-Rodbard-Chrambach (OMRC) model below.

Ogston-Morris-Rodbard-Chrambach (OMRC) Model

In the framework of the OMRC model, the low-field reduced mobility μ*(C_{RNP}) of an analyte is assumed to be equal to its free available volume f(C_{RNP}):

$${μ}^{*} ( {C}_{R N P} ) = \frac{μ}{{μ}_{0}} = f ( {C}_{R N P} )$$

where μ₀ is the free (no obstacle) mobility, and C_{RNP} is the non-activated RNP (obstacle concentration). For a specific system where analytes are considered to be spherical particles, the fractional volume available to the analyte is given by f(C_{RNP})=e^{-βC}^{_{RNP}}, where β is the retardation coefficient. Hence, electrophoretic mobility of the reporters can be extracted by fitting an exponential curve to our experimental results as:

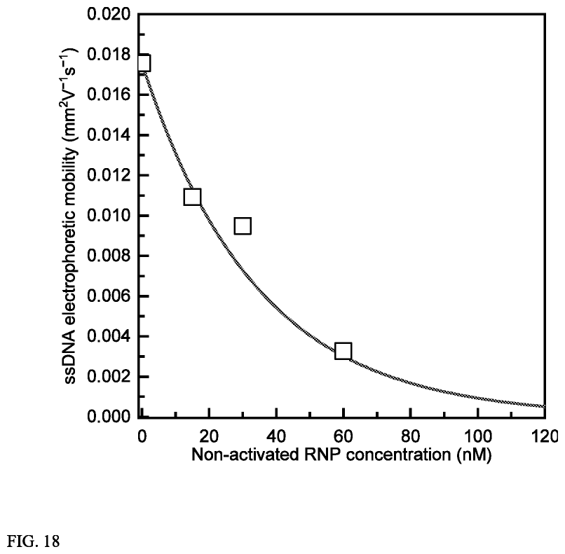

μ=μ₀e^{-βC}^{_{RNP}}

where extracted μ₀ and β from the fitting exponential line are 1.73×10⁻⁸ m² V⁻¹ s⁻¹ and 0.025 nM⁻¹, respectively, see FIG. 18.

Experimentally observed events for negative and positive case would follow the Poisson distribution with expected values of λₙ and μₚ respectively. As illustrated in FIG. 11A, an aim is to determine λₚ such that the overlap of Poisson probability density functions (PDF) with that of the negative case of λₙ is less than 5% (i.e., 95% confidence level for making a positive/negative call). For any fixed nanopore reading time Tₘ, one can solve for the maximal λₚ and therefore the minimal required reaction time Tᵣ. FIG. 11B plots the total experimental time (Tᵣ+Tₘ) as a function of HIV target nucleic acid and RNP concentrations. Three features were observed as follows, First, under a fixed RNP concentration, the total experimental time can be significantly reduced when HIV target nucleic acid concentration is increased up to that of RNP concentration, beyond which the total experimental time is independent of the HIV-1 target nucleic acid concentration. This is because the rate constant k is determined by the smaller amount between HIV-1 target nucleic acid and RNP. Second, although in regions where RNP concentration is higher than HIV-1 target nucleic acid, the constant rate k is independent of RNP concentration, the total experimental time would be significantly increased when RNP target concentration is increased, especially more than 50 nM regions. This stems from the fact that the electrophoretic mobility of the reporters decays exponentially as the RNP concentration is increased. Hence, more time is needed in the nanopore reading to make a call at 95% confidence. Third, an optimized combination of HIV-1 target nucleic acid and RNP concentration is required such that a positive/negative call at 95% confidence can be made within an hour (dashed line). It was found that the RNP concentration between 10-100 nM and HIV target nucleic acid concentration higher than 10 nM would be the optimized range in this example. If the starting HIV-1 DNA concentration is less than 10 nM, a pre-amplification step before SCAN is highly desirable for quick turnaround.

Sequence-Specific Example—HIV

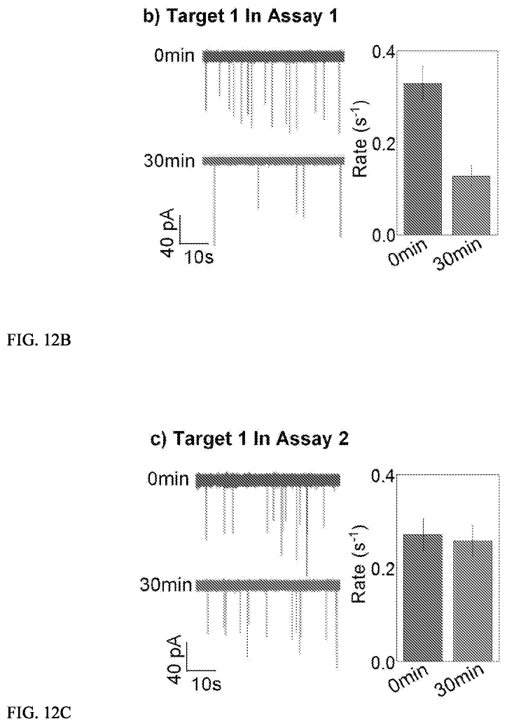

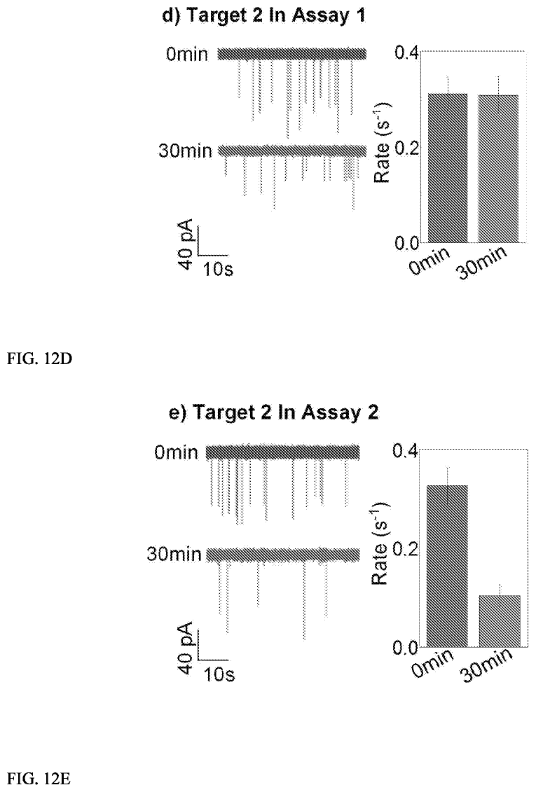

In this example, two sets of HIV-1 DNA targets and assays were used, in which each assay was specific to its target, Assay 1 was specific to Target 1 and Assay 2 was specific to Target 2. To test the cross-reactivity of designed assays, gel analysis was performed on the assay products, FIG. 12A shows the gel image. Reporter cleavage was observed when the assays were directed to their specific target, and no reporter cleavage if the assay is non-specific to the target. To validate the specificity of the SCAN method according to aspects of the present disclosure, four different assay-target combinations were used in this example. FIGS. 12B, 12C, 12D, and 12E present the results produced before and after 30 min of reaction. For Target 1 in Assay 1, the translocation event rate change is significant, from 0.329±0.036 s⁻¹ to 0.128±0.022 s⁻¹, whereas for Target 1 in Assay 2, the event rate change is negligible, from 0.271±0.034 s⁻¹ to 0.258±0.032 s⁻¹. Similarly, for Target 2 in Assay 1, the translocation event rate change is negligible, from 0.310±0.035 s⁻¹ to 0.308×0.039 s⁻¹, whereas Target 2 in Assay 2, the translocation event rate change is significant, from 0.326±0.037 s⁻¹ to 0.103±0.022 s⁻¹. It is clear that only the matched Cas12a assay and its target can produce a significant reduction in the number of translocation events after 30 minutes of reaction. These results demonstrate the SCAN method according to aspects of the present disclosure detects targets specifically.

Sequence-Specific Example—SARS-Cov-2

In this example, to detect SARS-CoV-2 virus, SARS-CoV-2 virus genome RNA is first converted to DNA and amplified via reverse transcription-recombinase polymerase amplification (RT-RPA) or RT-Loop-mediated isothermal amplification (RT-LAMP). The following PCR primers can be used to amplify SARS-CoV-2 virus nucleic acid of a sample prior to nanopore detection analysis: Forward: ttacaaacattggccgcaaa (SEQ ID NO:8), Reverse: gcgcgacattccgaagaa (SEQ ID NO:9)

Then A.s.Cas12a Ultra (IDT #10001272) Cas12a enzyme with its corresponding crRNA (cccccagcgcttcagcgttc (SEQ ID NO:10) with a PAM tttg) targeting SARS-CoV-2 N gene are contacted to form a non-activated RNP complex by incubation in 1×PBS at room temperature for 20 min. The non-activated RNP complex is then mixed with the amplified samples and incubated at 37° C. for 10 min for RNP activation to form activated RNP complex. A ssDNA reporter is then included in the mixture and incubated at 37° C. for cleavage of the reporter if SARS-CoV-2 N gene target nucleic acid. A solution of high salt concentration, over 1M, is then added to stop the reaction. This mixture is then loaded into the first chamber of a nanopore system as described herein for detection. Correct recognition of SARS-CoV-2 virus will indiscriminately cut the reporter nucleic acids, yielding fewer reads in the nanopore system.

Items

Item 1. A method of detecting an analyte in a solution, comprising: providing a nanopore counting device comprising; a first chamber and a second chamber; a barrier having a nanopore opening defined therein, the barrier separating the first chamber from the second chamber; a control/sensing system operable to apply an electrical potential between a solution in the first chamber and a solution in the second chamber and to sense a voltage and/or current between the chambers; disposing an ion-containing solution in the first and second chambers; disposing a sample in the first chamber, the sample containing or suspected of containing the analyte; applying an electrical potential between the solution in the first chamber and the solution in the second chamber; and sensing a baseline current between the chambers and detecting resistive pulses, wherein detecting resistive pulses provides a signal representative of presence of the analyte in the sample, and wherein no calibration step is performed before disposing the sample.

Item 2. The method of item 1, wherein sensing a baseline current between the chambers and detecting resistive pulses provides a count of a number of molecules of analyte that pass through the nanopore during a period of time.

Item 3. The method of item 2, further comprising determining an estimated concentration of the molecules of analyte in the first chamber, wherein determining comprises calculation using the formula:

$${C}_{mol} = \frac{Λ R}{μ {N}_{A} {I}_{b}} {C}_{ion}$$

where:
Λ is the molar conductivity of the buffer solution in Siemens per meter per mole;
R is the translocation rate in resistive pulses per second;
μ is the free solution electrophoretic mobility of the analyte in meters per volt second;
N_{A} is the Avogadro constant; and
I_{b} is the baseline current in nA.

Item 4. The method according to any of items 1 to 3, wherein the barrier with the nanopore is a solid state nanopore barrier.

Item 5. The method according any of items 1 to 4, wherein the nanopore has an opening size larger than a size of an analyte molecule, the nanopore opening size being within one order of magnitude of the size of the analyte molecule.

Item 6. The method according to any of items 2 to 5, wherein the period of time is determined by the number of resistive pulse, the number of resistive pulse during the period of time being at least 100.

Item 7. The method according to any of items 1 to 6, wherein the number of resistive pulses is at least 200.

Item 8. The method according to any of items 2 to 7, wherein the number of resistive pulses during the period of time is at least 200.

Item 9. The method according to any of items 1 to 8, wherein the analyte is a target nucleic acid sequence, and further comprising: providing a reporter nucleic acid; providing a CRISPR-Cas system guide RNA (crRNA) that hybridizes to the target nucleic acid sequence; providing a CRISPR enzyme, wherein the CRISPR enzyme is a Cas enzyme capable of forming a non-activated ribonucleoprotein (RNP) complex (non-activated RNP) with the guide RNA, and wherein the non-activated RNP complex is capable of binding to the target nucleic acid sequence, forming an activated RNP complex (activated RNP) having “trans” activity to cleave the reporter nucleic acid; contacting the crRNA and Cas enzyme, thereby forming the non-activated RNP; disposing the non-activated RNP in the first chamber with the sample, wherein the non-activated RNP and the target nucleic acid sequence specifically bind if the target nucleic acid is present in the sample, forming an activated RNP, wherein the activated RNP cleaves the reporter nucleic acid, and wherein cleavage of the reporter nucleic acid reduces passage of the reporter nucleic acid through the nanopore such that a reduction of resistive pulses is produced which provides a signal representative of presence of the target nucleic acid sequence in the sample.

Item 10. The method according to item 9, wherein the target nucleic acid sequence is DNA and the Cas enzyme is a Cas12 enzyme.

Item 11. The method according to item 9, wherein the target nucleic acid sequence is RNA and the Cas enzyme is a Cas13 enzyme.

Item 12. The method according to any of items 9 to 11, wherein the reporter nucleic acid is a linear or circular single-stranded DNA molecule.

Item 13. The method according to any of items 9 to 12, wherein the reporter nucleic acid does not include a label.

Item 14. The method according to any of items 9 to 13, wherein the target nucleic acid sequence is a nucleic acid of a microorganism.

Item 15. The method of any of items 9 to 14, wherein the target nucleic acid sequence is a nucleic acid of a virus, a bacterium, a fungus, or a parasite.

Item 16. The method of any of items 9 to 15, wherein the sample is obtained from a mammal.

Item 17. The method of any of items 9 to 16, wherein the sample is derived from a human.

Item 18. The method of any of items 9 to 17, wherein the sample is derived from a mammal having, or suspected of having, an infection by a virus, a bacterium, a fungus, or a parasite.

Item 19. The method of any of items 9 to 18, wherein the sample is derived from a human having, or suspected of having, an infection by a virus, a bacterium, a fungus, or a parasite.

Item 20. The method of any of items 9 to 15, wherein the sample is an environmental sample, containing, or suspected of containing, a virus, a bacterium, a fungus, or a parasite.

Item 21. The method of any of items 9 to 20, wherein the target nucleic acid sequence is a nucleic acid of a human immunodeficiency virus.

Item 22. The method of any of items 9 to 20, wherein the target nucleic acid sequence is a nucleic acid of a coronavirus.

Item 23. The method of item 22, wherein the coronavirus is a Sars-Cov-2 coronavirus.

Item 24. The method of any of items 9 to 15, wherein the sample is derived from a plant.

Item 25. The method of any of items 9 to 15 or 24, wherein the sample is derived from a plant having, or suspected of having, an infection by a virus, a bacterium, a fungus, or a parasite.

Item 26. A method of detecting a target nucleic acid sequence in a solution, comprising: providing a nanopore counting device comprising; a first chamber and a second chamber; a barrier having a nanopore opening defined therein, the barrier separating the first chamber from the second chamber; a control/sensing system operable to apply an electrical potential between a solution in the first chamber and a solution in the second chamber and to sense a voltage and/or current between the chambers; calibrating the nanopore counting device to determine a rate of translocation of molecules of a calibrant from a calibration solution through the nanopore when a calibrating electrical potential is applied between the chambers; disposing an ion-containing solution in the first and second chambers; providing a reporter nucleic acid; providing a CRISPR-Cas system guide RNA (crRNA) that hybridizes to the target nucleic acid sequence; providing a CRISPR enzyme, wherein the CRISPR enzyme is a Cas enzyme capable of forming a non-activated ribonucleoprotein (RNP) complex (non-activated RNP) with the guide RNA, and wherein the non-activated RNP is capable of binding to the target nucleic acid sequence, forming an activated RNP complex (activated RNP) having “trans” activity to cleave the reporter nucleic acid; contacting the crRNA and Cas enzyme, thereby forming the non-activated RNP; disposing the non-activated RNP in the first chamber with the sample, wherein the non-activated RNP and the target nucleic acid sequence specifically bind if the target nucleic acid is present in the sample, forming an activated RNP, wherein the activated RNP cleaves the reporter nucleic acid; applying an electrical potential between the solution in the first chamber and the solution in the second chamber; sensing current between the first chamber and the second chamber and detecting resistive pulses, wherein cleavage of the reporter nucleic acid reduces passage of the reporter nucleic acid through the nanopore such that a reduction of resistive pulses is produced which provides a signal representative of presence of the target nucleic acid sequence in the sample

Item 27. The method according to item 26, wherein:

the step of detecting resistive pulses further comprises counting resistive pulses to determine a number of reporter nucleic acid molecules that pass through the nanopore during a period of time, thereby determining a rate of translocation of the reporter nucleic acid molecules.

Item 28. The method according to item 26, further comprising determining the estimated concentration of the target nucleic acid sequence in the first chamber based on the reporter nucleic acid translocation rate as compared to the calibrant translocation rate.

Item 29. The method according to any of items 26 to 28, wherein the calibrating step comprises: disposing an ion-containing solution in the first and second chamber and a known concentration of calibrant molecules in the first chamber, the calibrant molecules being the same or similar to the reporter nucleic acid molecules; applying the calibrating electrical potential between the chambers; sensing current between the chambers and counting resistive pulses to determine a number of molecules of the calibrant that pass through the nanopore during a period of time; and determining a rate of translocation for the known concentration of calibrant at the calibrating electrical potential.

Item 30. The method according to any of items 26 to 29, wherein the barrier with the nanopore barrier is a solid state nanopore barrier or a biological nanopore barrier.

Item 31. The method according to any of items 26 to 30, wherein the calibrant molecules are the same as the reporter nucleic acid molecules, the calibrating electrical potential is in the range of 0.5 to 2 times the electrical potential used after the calibrating step, and the ion-containing solution during is the same during the calibrating step and after the calibrating step.

Item 32. The method according to any of items 26 to 31, wherein the target nucleic acid sequence is DNA and the Cas enzyme is a Cas12 enzyme.

Item 33. The method any of items 26 to 31, wherein the target nucleic acid sequence is RNA and the Cas enzyme is a Cas13 enzyme.

Item 34. The method any of items 26 to 33, wherein the reporter nucleic acid is a circular or linear single-stranded DNA molecule.

Item 35. The method any of items 26 to 34, wherein the reporter nucleic acid does not include a label.

Item 36. The method any of items 26 to 35, wherein the target nucleic acid sequence is a nucleic acid of a microorganism.

Item 37. The method of any of items 26 to 36, wherein the target nucleic acid sequence is a nucleic acid of a virus, a bacterium, a fungus, or a parasite.

Item 38. The method of any of items 26 to 37, wherein the sample is obtained from a mammal.

Item 39. The method of any of items 26 to 38, wherein the sample is derived from a human.

Item 40. The method of any of items 26 to 39, wherein the sample is derived from a mammal having, or suspected of having, an infection by a virus, a bacterium, a fungus, or a parasite.

Item 41. The method of any of items 26 to 40, wherein the sample is derived from a human having, or suspected of having, an infection by a virus, a bacterium, a fungus, or a parasite.

Item 42. The method of any of items 26 to 37, wherein the sample is an environmental sample, containing, or suspected of items, a virus, a bacterium, a fungus, or a parasite.

Item 43. The method of any of items 26 to 42, wherein the target nucleic acid sequence is a nucleic acid of a human immunodeficiency virus.

Item 44. The method of any of items 26 to 42, wherein the target nucleic acid sequence is a nucleic acid of a coronavirus.

Item 45. The method of item 44, wherein the coronavirus is a Sars-Cov-2 coronavirus.

Item 46. The method of any of items 26 to 37, wherein the sample is derived from a plant.

Item 47. The method of any of items 26 to 37 or 46, wherein the sample is derived from a plant having, or suspected of having, an infection by a virus, a bacterium, a fungus, or a parasite.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1.-25. (canceled)

26. A method of detecting a target nucleic acid sequence in a solution, comprising:
providing a nanopore counting device comprising;
a first chamber and a second chamber;
a barrier having a nanopore opening defined therein, the barrier separating the first chamber from the second chamber;
a control/sensing system operable to apply an electrical potential between a solution in the first chamber and a solution in the second chamber and to sense a voltage and/or current between the chambers;
calibrating the nanopore counting device to determine a rate of translocation of molecules of a calibrant from a calibration solution through the nanopore when a calibrating electrical potential is applied between the chambers;
disposing an ion-containing solution in the first and second chambers;
providing a reporter nucleic acid;
providing a CRISPR-Cas system guide RNA (crRNA) that hybridizes to the target nucleic acid sequence;
providing a CRISPR enzyme, wherein the CRISPR enzyme is a Cas enzyme capable of forming a non-activated ribonucleoprotein (RNP) complex (non-activated RNP) with the guide RNA, and wherein the non-activated RNP is capable of binding to the target nucleic acid sequence, forming an activated RNP complex (activated RNP) having “trans” activity to cleave the reporter nucleic acid;
contacting the crRNA and Cas enzyme, thereby forming the non-activated RNP;
disposing the non-activated RNP in the first chamber with the sample, wherein the non-activated RNP and the target nucleic acid sequence specifically bind if the target nucleic acid is present in the sample, forming an activated RNP, wherein the activated RNP cleaves the reporter nucleic acid;
applying an electrical potential between the solution in the first chamber and the solution in the second chamber;
sensing current between the first chamber and the second chamber and detecting resistive pulses, wherein cleavage of the reporter nucleic acid reduces passage of the reporter nucleic acid through the nanopore such that a reduction of resistive pulses is produced which provides a signal representative of presence of the target nucleic acid sequence in the sample

27. The method according to claim 26, wherein:
the step of detecting resistive pulses further comprises counting resistive pulses to determine a number of reporter nucleic acid molecules that pass through the nanopore during a period of time, thereby determining a rate of translocation of the reporter nucleic acid molecules.

28. The method according to claim 26, further comprising determining the estimated concentration of the target nucleic acid sequence in the first chamber based on the reporter nucleic acid translocation rate as compared to the calibrant translocation rate.

29. The method according to claim 26, wherein the calibrating step comprises:
disposing an ion-containing solution in the first and second chamber and a known concentration of calibrant molecules in the first chamber, the calibrant molecules being the same or similar to the reporter nucleic acid molecules;
applying the calibrating electrical potential between the chambers;
sensing current between the chambers and counting resistive pulses to determine a number of molecules of the calibrant that pass through the nanopore during a period of time; and
determining a rate of translocation for the known concentration of calibrant at the calibrating electrical potential.

30. The method according to claim 26, wherein the barrier with the nanopore barrier is a solid state nanopore barrier or a biological nanopore barrier.

31. The method according to claim 26, wherein the calibrant molecules are the same as the reporter nucleic acid molecules, the calibrating electrical potential is in the range of 0.5 to 2 times the electrical potential used after the calibrating step, and the ion-containing solution during is the same during the calibrating step and after the calibrating step.

32. The method according to claim 26, wherein the target nucleic acid sequence is DNA and the Cas enzyme is a Cas12 enzyme.

33. The method according to claim 26, wherein the target nucleic acid sequence is RNA and the Cas enzyme is a Cas13 enzyme.

34. The method according to claim 26, wherein the reporter nucleic acid is a circular or linear single-stranded DNA molecule.

35. The method according to claim 26, wherein the reporter nucleic acid does not include a label.

36. The method according to claim 26, wherein the target nucleic acid sequence is a nucleic acid of a microorganism.

37. The method according to claim 26, wherein the target nucleic acid sequence is a nucleic acid of a virus, a bacterium, a fungus, or a parasite.

38. The method according to claim 26, wherein the sample is obtained from a mammal or plant.

39. The method according to claim 26, wherein the sample is derived from a human.

40. The method according to claim 26, wherein the sample is derived from a mammal or plant having, or suspected of having, an infection by a virus, a bacterium, a fungus, or a parasite.

41. The method according to claim 26, wherein the sample is derived from a human having, or suspected of having, an infection by a virus, a bacterium, a fungus, or a parasite.

42. The method according to claim 26, wherein the sample is an environmental sample, containing, or suspected of containing, a virus, a bacterium, a fungus, or a parasite.

43. The method according to claim 26, wherein the target nucleic acid sequence is a nucleic acid of a human immunodeficiency virus or coronavirus.

44. (canceled)

45. The method according to claim 43, wherein the coronavirus is a Sars-Cov-2 coronavirus.

46.-47. (canceled)

48. The method according to claim 26, further comprising amplifying the target nucleic acid sequence before adding the sample to the first chamber.
